(19) United States
(12) Patent Application Publication
(10) Pub. No.: US 2003/0207804 A1
(43) Pub. Date: Nov. 6, 2003

(54) Modified peptide nucleic acids

(76) Inventors: Muthiah Manoharan, Carlsbad, CA (US); Kallanthottathil G. Rajeev, Vista, CA (US)

Correspondence Address: WOODCOCK WASHBURN LLP, ONE LIBERTY PLACE, 46TH FLOOR, 1650 MARKET STREET, PHILADELPHIA, PA, 19103, US

(21) Appl. No.: 10/155,920

(22) Filed: May 24, 2002

Related U.S. Application Data
(60) Provisional application No. 60/293,592, filed on May 25, 2001.

(51) Int. Cl. A61K 48/00; C07K 7/08; C07K 7/06

(52) U.S. Cl. 514/12; 530/324; 530/325; 530/326; 514/13; 514/14

(57) ABSTRACT

The present peptide nucleic acids exhibit enhanced cellular uptake and distribution. The peptide nucleic acids of the invention comprise naturally-occurring nucleobases and non-naturally-occurring nucleobases attached to a polyamide backbone. Non-naturally-occurring bases include monocyclic, bi-cyclic, and tricyclic heterocycles. Modified backbones are also provided.

### FIELD OF THE INVENTION

The present invention is directed to compositions comprising novel peptide nucleic acids (PNA) composed of naturally-occuring nucleobases and non-naturally-occuring nucleobases which are bound to a polyamide backbone. The PNA compositions of the present invention exhibit enhanced properties including but not limited to cellular uptake and distribution.

### BACKGROUND OF THE INVENTION

The function of a gene starts by transcription of its information to a messenger RNA (mRNA). By interacting with the ribosomal complex, MRNA directs synthesis of proteins. This protein synthesis process is known as translation. Translation requires the presence of various cofactors, building blocks, amino acids and transfer RNAs (tRNAs), all of which are present in normal cells.

Most conventional drugs exert their effect by interacting with and modulating one or more targeted endogenous proteins, e.g., enzymes. Typically, however, such drugs are not specific for targeted proteins but interact with other proteins as well. Thus, use of a relatively large dose of drug is necessary to effectively modulate the action of a particular protein. If the modulation of a protein activity could be achieved by interaction with or inactivation of MRNA, a dramatic reduction in the amount of drug necessary and in the side-effects of the drug could be achieved. Further reductions in the amount of drug necessary and the side-effects could be obtained if such interaction is site-specific. Since a functioning gene continually produces mRNA, it would be even more advantageous if gene transcription could be arrested in its entirety.

Oligonucleotides and their analogs have been developed and used as diagnostics, therapeutics and research reagents. One example of a modification to oligonucleotides is labeling with non-isotopic labels, e.g., fluorescein, biotin, digoxigenin, alkaline phosphatase, or other reporter molecules. Other modifications have been made to the ribose phosphate backbone to increase the resistance to nucleases. These modifications include use of linkages such as methyl phosphonates, phosphorothioates and phosphorodithioates, and 2′-O-methyl ribose sugar moieties. Other oligonucleotide modifications include those made to modulate uptake and cellular distribution. Phosphorothioate oligonucleotides are presently being used as antisense agents in human clinical trials for the treatment of various disease states. Although some improvements in diagnostic and therapeutic uses have been realized with these oligonucleotide modifications, there exists an ongoing demand for improved oligonucleotide analogs.

There are several known nucleic acid analogs having nucleobases bound to backbones other than the naturally-occurring ribonucleic acids or deoxyribonucleic acids. These nucleic acid analogs have the ability to bind to nucleic acids with complementary nucleobase sequences. Among these, the peptide nucleic acids (PNAs), as described, for example, in WO 92/20702, have been shown to be useful as therapeutic and diagnostic reagents. This may be due to their generally higher affinity for complementary nucleobase sequence than the corresponding wild-type nucleic acids.

PNAs are useful surrogates for oligonucleotides in binding to DNA and RNA. Egholm et al., Nature, 1993, 365, 566, and references cited therein. The current literature reflects the various applications of PNAs. Hyrup et al., Bioorganic & Med. Chem., 1996, 4, 5; and Nielsen, Perspectives Drug Disc. Des., 1996, 4, 76.

PNAs are compounds that are analogous to oligonucleotides, but differ in composition. In PNAs, the deoxyribose backbone of oligonucleotide is replaced by a peptide backbone. Each subunit of the peptide backbone is attached to a naturally-occurring or non-naturally-occurring nucleobase. One such peptide backbone is constructed of repeating units of N-(2-aminoethyl)glycine linked through amide bonds. The synthesis of PNAs via preformed monomers was previously described in WO 92/20702 and WO 92/20703, the contents of which are herein incorporated by reference. More recent advances in the structure and synthesis of PNAs are illustrated in WO 93/12129 and U.S. Pat. No. 5,539,082, issued Jul. 23, 1996, the contents of both being herein incorporated by reference. Further, the literature is replete with publications describing synthetic procedures, biological properties and uses of PNAs. For example, PNAs possess the ability to effect strand displacement of double-stranded DNA. Patel, Nature, 1993, 365, 490. Improved synthetic procedures for PNAs have also been described. Nielsen et al., Science, 1991, 254, 1497; and Egholm, J. Am. Chem. Soc., 1992, 114, 1895. PNAs form duplexes and triplexes with complementary DNA or RNA. Knudson et al., Nucleic Acids Research, 1996, 24, 494; Nielsen et al., J. Am. Chem. Soc., 1996, 118, 2287; Egholm et al., Science, 1991, 254, 1497; Egholm et al., J. Am. Chem. Soc., 1992, 114, 1895; and Egholm et al., J. Am. Chem. Soc., 1992, 114, 9677.

PNAs bind to both DNA and RNA and form PNA/DNA or PNA/RNA duplexes. The resulting PNA/DNA or PNA/RNA duplexes are bound tighter than corresponding DNA/DNA or DNA/RNA duplexes as evidenced by their higher melting temperatures (Tₘ). This high thermal stability of PNA/DNA(RNA) duplexes has been attributed to the neutrality of the PNA backbone, which results elimination of charge repulsion that is present in DNA/DNA or RNA/RNA duplexes. Another advantage of PNA/DNA(RNA) duplexes is that Tₘ is practically independent of salt concentration. DNA/DNA duplexes, on the other hand, are highly dependent on the ionic strength.

Triplex formation by oligonucleotides has been an area of intense investigation since sequence-specific cleavage of double-stranded deoxyribonucleic acid (DNA) was demonstrated. Moser et al., Science, 1987, 238, 645. The potential use of triplex-forming oligonucleotides in gene therapy, diagnostic probing, and other biomedical applications has generated considerable interest. Uhlmann et al., Chemical Reviews, 1990, 90, 543. Pyrimidine oligonucleotides have been shown to form triple helix structures through binding to homopurine targets in double-stranded DNA. In these structures the new pyrimidine strand is oriented parallel to the purine Watson-Crick strand in the major groove of the DNA and binds through sequence-specific Hoogsteen hydrogen bonding. The sequence specificity is derived from thymine recognizing adenine (T:A-T) and protonated cytosine recognizing guanine (C⁺:G-C). Best et al., J. Am. Chem. Soc., 1995, 117, 1187. In a less well-studied triplex motif, purine-rich oligonucleotides bind to purine targets of double-stranded DNA. The orientation of the third strand in this motif is anti-parallel to the purine Watson-Crick strand, and the specificity is derived from guanine recognizing guanine (G:G-C) and thymine or adenine recognizing adenine (A:A-T or T:A-T). Greenberg et al., J. Am. Chem. Soc., 1995, 117, 5016.

Homopyrimidine PNAs have been shown to bind complementary DNA or RNA forming (PNA)₂/DNA(RNA) triplexes of high thermal stability. Egholm et al., Science, 1991, 254, 1497; Egholm et al., J. Am. Chem. Soc., 1992, 114, 1895; Egholm et al., J.

Am. Chem. Soc., 1992, 114, 9677. The formation of triplexes involving two PNA strands and one nucleotide strand has been reported in U.S. patent application Ser. No. 08/088,661, filed Jul. 2, 1993, the contents of which are incorporated herein by reference. The formation of triplexes in which the Hoogsteen strand is parallel to the DNA purine target strand is preferred to formation of anti-parallel complexes. This allows for the use of bis-PNAs to obtain triple helix structures with increased pH-independent thermal stability using pseudoisocytosine instead of cytosine in the Hoogsteen strand. Egholm et al., J. Am. Chem. Soc., 1992, 114, 1895. Further, see WO 96/02558, the contents of which are incorporated herein by reference.

Peptide nucleic acids have been shown to have higher binding affinities (as determined by their Tm's) for both DNA and RNA than that of DNA or RNA to either DNA or RNA. This increase in binding affinity makes these peptide nucleic acid oligomers especially useful as molecular probes and diagnostic agents for nucleic acid species.

In addition to increased affinity, PNAs have increased specificity for DNA binding. Thus, a PNA/DNA duplex mismatch show 8 to 20□C drop in the Tₘ relative to the DNA/DNA duplex. This decrease in Tₘ is not observed with the corresponding DNA/DNA duplex mismatch. Egholm et al., Nature 1993, 365, 566.

A further advantage of PNAs, compared to oligonucleotides, is that the polyamide backbone of PNAs is resistant to degradation by enzymes.

Considerable research is being directed to the application of oligonucleotides and oligonucleotide analogs that bind to complementary DNA and RNA strands for use as diagnostics, research reagents and potential therapeutics. For many applications, the oligonucleotides and oligonucleotide analogs must be transported across cell membranes or taken up by cells to express their activity.

Recent research efforts are described in, for example, Eldrup et al., Eur. J. Org. Chem. 2001, 1781-1790; Hickman et al., Chem. Commun., 2000, 2251-2252; Wilhelmsson et al., J. Am. Chem. Soc., 2001, 123, 2434-2435; Sanjayan et al., Organic Letters, 2000, 2(18), 2825-2828; Okamoto et al., Organic Letters, 2001, 3(6), 925-927; Egholm et al., Nucleic Acids Research, 1995, 23(2), 217-222; Haaima et al., Nucleic Acids Research, 1997, 25(22), 4639-4643; Eldrup et al., J. Am. Chem. Soc., 1997, 119, 11116-11117; Clivio et al., J. Am. Chem. Soc., 1998, 120, 1157-1166; Ikeda et al, Tetrahedron Letters., 2001, 42, 2529-2531; Challa et al., Tetrahedron Letters., 1999, 40, 419-422; Challa et al., Tetrahedron Letters., 1999, 40, 8333-8336; Challa et al., Organic Letters, 1991, 1(10), 1639-1641; Ferrer et al., Bioorganic & Medicinal Chemistry, 2000, 8, 291-297; Eldrup et al., Eur. J. Org. Chem., 2001, 9, 1781-1790; Hickman et al., Chem. Commun. (Cambridge)., 2000, 22, 2251-2252; Puschl et al., J. Org. Chem., 2001, 66, 707-712; Hudson et al., Tetrahedron Letters., 2002, 43, 1381-1386; D'Costa et al., Organic Letters, 1999, 1(10), 1513-1516; D'Costa et al., Organic Letters, 2001, 3(9), 1281-1284; Kumar et al., Organic Letters, 2001, 3(9), 1269-1272; Jordan et al., Bioorganic & Medicinal Chemistry Letters, 1997, 7(6), 681-686; Lowe et al., Bioorganic Chemistry, 1997, 25, 321-329; Gangamani et al., Tetrahedron., 1999, 55, 177-192; Vilaivan et al., Bioorganic & Medicinal Chemistry Letters, 2000, 10, 2541-2545; Gangamani et al., Tetrahedron., 1996, 52(47), 15017-15030; Dueholm et al., New J. Chem., 1997, 21, 19-31, all of which are herein incorporated by reference in their entireties.

Other research efforts involve the monocyclic structures shown below in Table 1. Heterocyclic modifications of PNA as shown below in Table 2 are also the subject of research efforts. All of the references shown in Tables 1 and 2 are herein incorporated by reference in their entirety.

TABLE 1

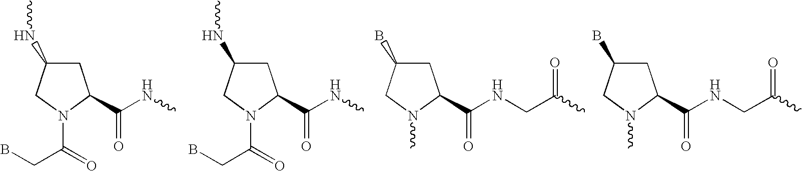

(Jordan et al., Bioorg. Med. Chem. Lett, 1997, 7, 681)

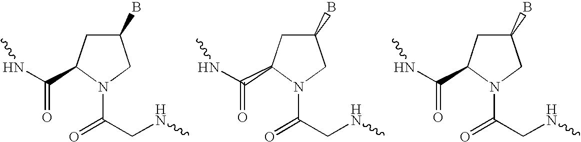

(Lowe et al, Bioorg. Chem. 1997, 25, 321)

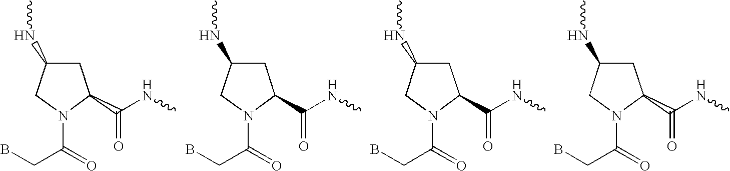

(Gangamani et. al, Tetrahedron, 1999, 55, 177)

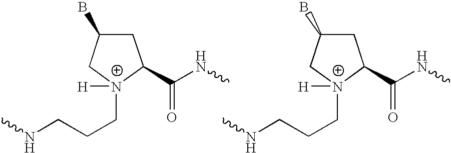

(D'Costa et al, Org Lett, 1999, 7, 1513)

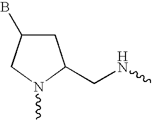

(Hickman et al, Chem Commun., 2000, (22), 2251)

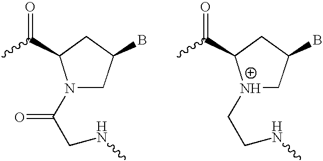

(Vilaivan et. al., Bioorg Med. Chem Lett, 2000, 10, 2541)

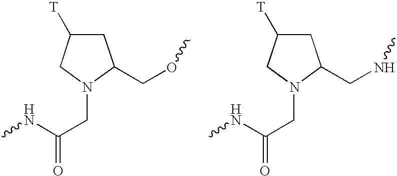

(Kumar et. al. Org. Lett., 2001, 3, 1269)

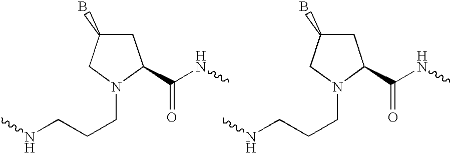

(D'Costa et al, Org Lett, 2001, 3, 1281)

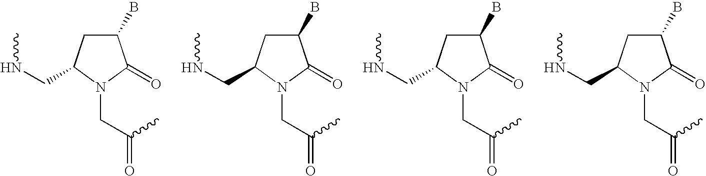

(Pusch1 et. al., J. Org. Chem, 2001, 66, 707)

TABLE 2

| | |
|---|---|
| 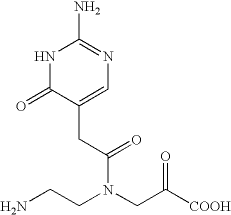 | 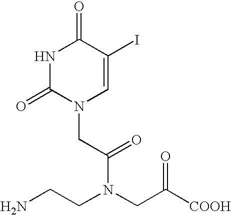 |
| (Eghorn et. al., Nucleic Acids Res., 1995, 23, 217) | (Betts et at, Science, 1995, 270, 1838) |
| 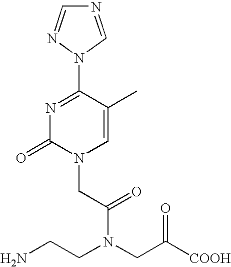 | 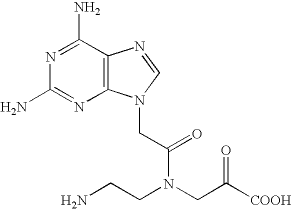 |
| (Breipohl et. al., Bioorg. Med. Chem. Lett., 1996, 6,685) | (Haaima et at, Nucleic Acids Res, 1997, 25,4639) |
| 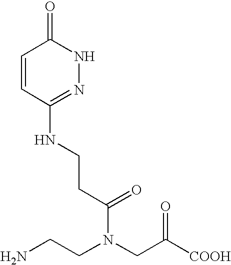 | 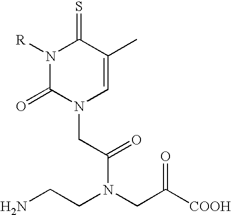 |
| (Eldrup et. al., J Am Chem Soc. 1997, 119, 11116) | R = H or CH₃, (Clivio et at., J Am Chem Soc. 1998, 120, 1157) |
| 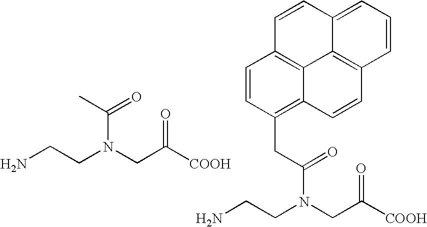 | 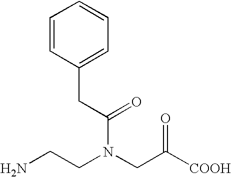 |
| (Challa and Woski, Tetrahedron Lett., 1999, 40, 419) | (Challa and Woski, Tetrahedron Lett., 1999, 40, 8333) |

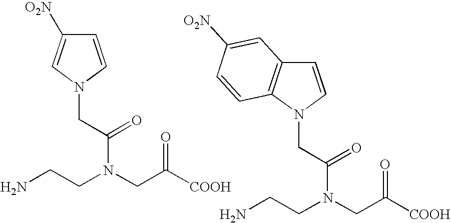

(Challa et. al, Organic Lett, 1999, 1, 1639)

| | |
|---|---|
| 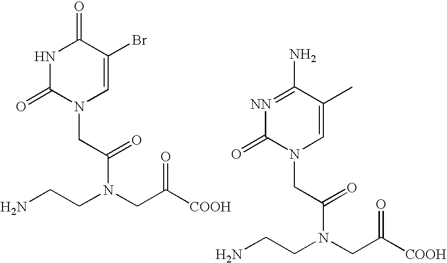 | 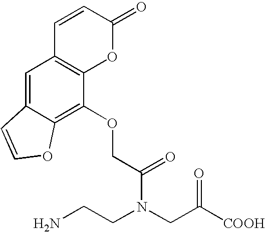 |
| (Ferrer et. al, Bioorg. Med. Chem, 2000, 8, 291) | (Okamoto et. al, Organi Lett, 2001, 3, 925) |

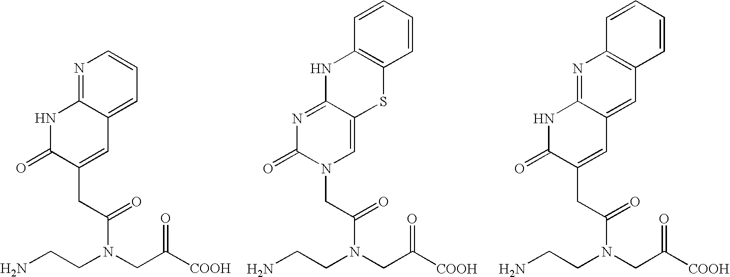

(Eldrup et at., Eur. J. Org. Chem., 2001, (9), 1781)

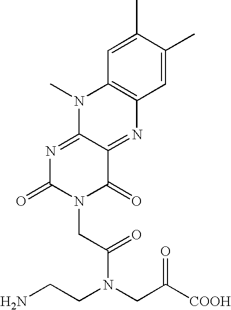

(Ikeda et. al., Tetrahedron Lett, 2001, 42, 2529)

Despite recent advances, there remains a need for stable compositions with enhanced cellular uptake and distribution.

### SUMMARY OF THE INVENTION

In one aspect, the present invention provides peptide nucleic acids having the structure:

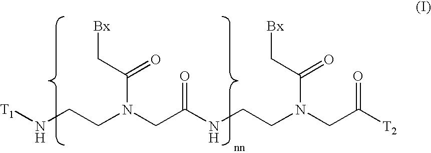

wherein:

T₁ is hydrogen, an amino protecting group, —C(O)R₅, substituted or unsubstituted C₁-C₁₀ alkyl, substituted or unsubstituted C₂-C₁₀ alkenyl, substituted or unsubstituted C₂-C₁₀ alkynyl, alkylsulfonyl, arylsulfonyl, a chemical functional group, a reporter group, a conjugate group, a D or L α-amino acid linked via the α-carboxyl group or optionally through the ω-carboxyl group when the amino acid is aspartic acid or glutamic acid or a peptide derived from D, L or mixed D and L amino acids linked through a carboxyl group, wherein the substituent groups are selected from hydroxyl, amino, alkoxy, carboxy, benzyl, phenyl, nitro, thiol, thioalkoxy, halogen, alkyl, aryl, alkenyl and alkynyl;

T₂ is —OH, —N(Z₁)Z₂, R₅, D or L α-amino acid linked via the α-amino group or optionally through the ω-amino group when the amino acid is lysine or ornithine or a peptide derived from D, L or mixed D and L amino acids linked through an amino group, a chemical functional group, a reporter group or a conjugate group;

nn is from 2 to about 50;

each Bx is, independently, an optionally protected heterocyclic base moiety wherein at least one of said heterocyclic base moieties has one of formulas II or III:

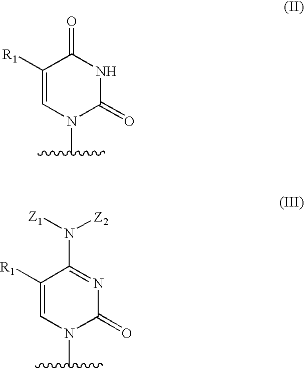

wherein:

R₁ is —CH₂—Q₁, —C≡C—Q₂, —CH₂—(CH₂)_{n—Q}₃, or —CH═CH—C(═O)—Q₄;

Q₁ is —N₃, —CN, —N(Z₁)Z₂, —N(Z₁)—(CH₂)ₙ—C(═NH₂)—N(H)—Z₃, —N(Z₁)—C(═J)—N(H)—Z₅, —L—(CH₂)n—C(═O)Z₃, —L—(CH₂),—L—Z₃, —L—(CH₂)ₙ—N(H)Z₁, —L—(CH₂)ₙ—N(Z₁)—(CH₂)ₙ—N(H)Z₁, —L—(CH₂)ₙ—C(═NH)N(Z₁)Z₃ or —L—(CH₂)ₙ—N(Z₁)—C(═J)—N(H)Z₃;

Q₂ is H, C₁-C₆ alkyl, —C(═O)—N(H)Z₁, —C(═O)—O—CH₂—CH₃, C(═O)—O—benzyl, —C(═O)—Z₄, —CH₂—O—Q₆, —CH₂—C(═NH)—N(H)—Z₃, —CH₂—N(H)—Z₂, —CH₂—N(H)—C(═O)—CF₃, —CH₂—N(H)Z₁, —CH₂—N(H)—C(═NH)—N(H)—Z₃, —CH₂—O—N(H)—C(═O)—(CH₂)ₙ—Q₅, —(═O)—N(H)—(CH₂)ₙ—Q₅ or —CH₂—N(H)—C(═O)—(CH₂)ₙ—Q₅;

Q₃ is hydrogen, —O—C₁-C₆ alkyl, —N(H)—Z₁, —N(H)—Z₂, —N(H)—C(═O)—CF₃, —N(H)—C(═NH)—N(H)Z₁, —O—Q₆, —N(H)—C(═O)—(CH₂)ₙ—Q₅, —O—N(H)—C(═O)—(CH₂)ₙ—Q₅, —C(═O)—N(H)—(CH₂)ₙ—Q₅ or —C(═O)—Q₇;

Q₄ is Z₄, —L—Z₃, —N(H)Z₃, —C(═NH)—N(H)Z₃, —N(H)—C(═J) N(H)Z₃, N(Z₁)—(CH₂)ₙ—N(H)Z₃, —C(═O)—N(H)—(CH₂)ₙ—C(═NH)—N(H)Z₃, —C(═O)—N(H)—(CH₂)ₙ—N(H)—C(═J)—N(H)Z₃ or —C(═O)—N(H)—(CH₂)ₙ—N(Z₁)—(CH₂)ₙ—N(H)Z₁;

Q₅ is —L—Z₃, —N(H)Z₃, —C(═NH)—N(H)Z₃, —N(H)—C(═J) N(H)Z₃ or N(Z₁)—(CH₂)ₙ—N(H)Z₃;

Q₆ is hydrogen, —N(H)Z₁, —N(H)Z₂, benzyl, benzoyl, —C(═O)—(CH₂)ₙ—H or phthalimido;

Q₇ is —OH, —O—C₁-C₆ alkyl, —O—-benzyl, —Z₄, —N(H)Z₁,

each L is O or S;

each J is O, S or NH;

each n is from 1 to 6;

Z₁ is hydrogen, C₁-C₆ alkyl, or an amino protecting group;

Z₂ is hydrogen, C₁-C₆ alkyl, an amino protecting group, —C(═O)—(CH₂)ₙ—J—Z₃, a D or L α-amino acid linked via the α-carboxyl group or optionally through the ω-carboxyl group when the amino acid is aspartic acid or glutamic acid or a peptide derived from D, L or mixed D and L amino acids linked through a carboxyl group;

Z₃ is hydrogen, an amino protecting group, —C₁-C₆ alkyl, —C(═O)—CH₃, benzyl, benzoyl, or —(CH₂)ₙ—N(H)Z₁;

Z₄ is a D or L α-amino acid linked via the α-amino group or optionally through the ω-amino group when the amino acid is lysine or ornithine or a peptide derived from D, L or mixed D and L amino acids linked through an amino group;

Z₅ is hydrogen, an amino protecting group or —C(═O)—(CH₂)ₙ—J—Z₃; and

each R₅ is a carbonyl protecting group.

In certain other embodiments the compounds of this invention have the structure of Formula (I) wherein:

T₁ is hydrogen, an amino protecting group, —C(O)R₅, substituted or unsubstituted C₁-C₁₀ alkyl, substituted or unsubstituted C₂-C₁₀ alkenyl, substituted or unsubstituted C₂-C₁₀ alkynyl, alkylsulfonyl, arylsulfonyl, a chemical functional group, a reporter group, a conjugate group, a D or L α-amino acid linked via the α-carboxyl group or optionally through the ω-carboxyl group when the amino acid is aspartic acid or glutamic acid or a peptide derived from D, L or mixed D and L amino acids linked through a carboxyl group, wherein the substituent groups are selected from hydroxyl, amino, alkoxy, carboxy, benzyl, phenyl, nitro, thiol, thioalkoxy, halogen, alkyl, aryl, alkenyl and alkynyl;

T₂ is —OH, —N(Z₁)Z₂, R₅, D or L α-amino acid linked via the ac-amino group or optionally through the ω-amino group when the amino acid is lysine or ornithine or a peptide derived from D, L or mixed D and L amino acids linked through an amino group, a chemical functional group, a reporter group or a conjugate group;

nn is from 2 to about 50;

each Bx is, independently, an optionally protected heterocyclic base moiety; wherein at least one of said heterocyclic base moieties has one of formulas V or VI:

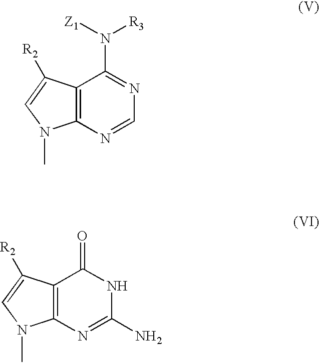

wherein:

R₂ is hydrogen and R₃ is Z₁, —C(═J)—N(H)Z₁, —C(═O)—(CH₂)ₙ—N(H)Z₁, —C(═O)—(CH₂)ₙ—L—Z₉, —(CH₂)ₙN(H)Z₁, —(CH₂)ₙ—N(Z₁, —(CH₂)ₙ—N(H)Z₁, —(CH₂)ₙ—N(H)—C(═J)—N(H)Z₃ or —(CH₂)ₙ—C(═NH)—N(H)Z₃;

or R₃ is hydrogen and R₂ is —C≡C—R₄ or —(CH₂)ₘ—R₄;

L is O or S;

J is O, S or NH;

m is from 2to 6;

each n is from 1 to 6;

R₄ is H, C₁-C₆ alkyl, —CH₂OH, —CH₂—O—Q₆, —CH₂—N(H)—C(═O)—CF₃, —CH₂—N(H)Z₁, —CH₂—N(H)Z₂, —C(═O)—Z₄, —C(═O)—N(H)—(CH₂)ₙ—Q₅, —CH₂—N(H)—C(═O)—(CH₂)ₙQ₅ or —CH₂—O—N(H)—C(═O)—(CH₂)ₙ—Q₅;

Q₅ is —L—Z₃, —N(H)Z₁, —C(═NH)—N(H)Z₃, —N(H)—C(═J) N(H)Z₃ or —N(Z₁)—(CH₂)ₙ—N(H)Z₁;

Q₆ is —N(H)Z₁, —N(H)Z₂, benzyl, benzoyl, —C(═O)—(CH₂)ₙ—CH₃ or phthalimido;

Z₁ is hydrogen, C₁-C₆ alkyl, or an amino protecting group;

Z₂ is hydrogen, C₁-C₆ alkyl, an amino protecting group, —C(═O)—(CH₂)ₙ—J—Z₃, a D or L α-amino acid linked via the α-carboxyl group or optionally through the ω-carboxyl group when the amino acid is aspartic acid or glutamic acid or a peptide derived from D, L or mixed D and L amino acids linked through a carboxyl group;

Z₃ is hydrogen, an amino protecting group, —C₁-C₆ alkyl, —C(═O)—CH₃, benzyl, benzoyl, or —(CH₂)ₙ—N(H)Z₁;

Z₄ is —OH, C₁-C₆ alkyl, benzyl, —N(H)Z₁, a D or L α-amino acid linked via the α-amino group or optionally through the ω-amino group when the amino acid is lysine or ornithing or a peptide derived from D, L or mixed D and L amino acids linked through an amino group;

Z₉ is hydrogen, —C₁-C₆ alkyl, —C(═O)—CH₃, benzyl or a D or L α-amino acid linked via the α-carboxyl group or optionally through the ω-carboxyl group when the amino acid is aspartic acid or glutamic acid or a peptide derived from D, L or mixed D and L amino acids linked through a carboxyl group; and

each R₅ is a carbonyl protecting group.

In other embodiments, this invention provides compounds having the structure Formula (I) wherein:

T₁ is hydrogen, an amino protecting group, —C(O)R₅, substituted or unsubstituted C₁-C₁₀ alkyl, substituted or unsubstituted C₂-C₁₀ alkenyl, substituted or unsubstituted C₂-C₁₀ alkynyl, alkylsulfonyl, arylsulfonyl, a chemical functional group, a reporter group, a conjugate group, a D or L α-amino acid linked via the α-carboxyl group or optionally through the ω-carboxyl group when the amino acid is aspartic acid or glutamic acid or a peptide derived from D, L or mixed D and L amino acids linked through a carboxyl group, wherein the substituent groups are selected from hydroxyl, amino, alkoxy, carboxy, benzyl, phenyl, nitro, thiol, thioalkoxy, halogen, alkyl, aryl, alkenyl and alkynyl;

T₂ is —OH, —N(Z₁)Z₂, R₅, D or L α-amino acid linked via the α-amino group or optionally through the ω-amino group when the amino acid is lysine or ornithine or a peptide derived from D, L or mixed D and L amino acids linked through an amino group, a chemical functional group, a reporter group or a conjugate group;

nn is from 2 to about 50;

each Bx is, independently, an optionally protected heterocyclic base moiety wherein at least one of said heterocyclic base moieties has formula VIII:

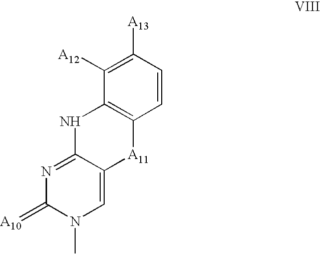

wherein

A₁₀ is S; and A₁₁ is CH₂, O or S; or

A₁₀ is O and A₁₁ is CH₂;

one of A₁₂ and A₁₃ is hydrogen and the other of A₁₂ and A₁₃ is a group of formula:

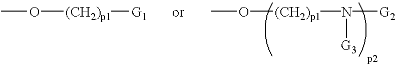

wherein:

G₁ is —CN, —OA₂₀, —SA₂₀, —N(H)A₂₀, —ON(H)A₂₀ or —C(═NH)N(H)A₂₀;

G₂ is H, —NHA₂₀, —C(═O)N(H)A₂₀, —C(═S)N(H)A₂₀ or —C(═NH)N(H)A₂₀,

each G₃ is, independently, H or an amino protecting group;

A₂₀ is H, a protecting group, substituted or unsubstituted C₁-C₁₀ alkyl, acetyl, benzyl, —(CH₂)ₚ₃NH₂, —(CH₂)ₚ₃N(H)G₃, a D or L α-amino acid, or a peptide derived from D, L or racemic α-amino acids;

each R₅ is a carbonyl protecting group;

each p1 is, independently, from 2 to about 6;

p2 is from 1 to about 3; and

p3 is from 1 to about 4.

In other embodiments, the instant invention provides compounds having the structure Formula (I) wherein:

T₁ is hydrogen, an amino protecting group, —C(O)R₅, substituted or unsubstituted C₁-C₁₀ alkyl, substituted or unsubstituted C₂-C₁₀ alkenyl, substituted or unsubstituted C₂-C₁₀ alkynyl, alkylsulfonyl, arylsulfonyl, a chemical functional group, a reporter group, a conjugate group, a D or L α-amino acid linked via the α-carboxyl group or optionally through the ω-carboxyl group when the amino acid is aspartic acid or glutamic acid or a peptide derived from D, L or mixed D and L amino acids linked through a carboxyl group, wherein the substituent groups are selected from hydroxyl, amino, alkoxy, carboxy, benzyl, phenyl, nitro, thiol, thioalkoxy, halogen, alkyl, aryl, alkenyl and alkynyl;

T₂ is —OH, —N(Z₁)Z₂, R₅, D or L α-amino acid linked via the α-amino group or optionally through the ω-amino group when the amino acid is lysine or ornithine or a peptide derived from D, L or mixed D and L amino acids linked through an amino group, a chemical functional group, a reporter group or a conjugate group;

nn is from 2 to about 50;

each Bx is, independently, an optionally protected heterocyclic base moiety wherein at least one of said heterocyclic base moieties has formula XVI:

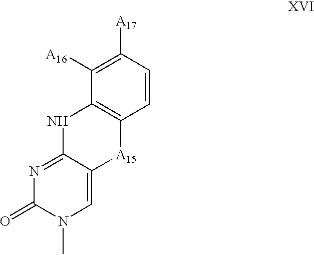

wherein

A₁₅ is O or S; and

A₁₆ is selected from the group consisting of —O—(CH₂)ₚ₁C(═NH)N(H)A₂₀, —O—(CH₂)ₚ₁N(H)—C(═O)N(H)A₂₀ or —O—(CH₂)ₚ₁N(H)—C(═S)N(H)A₂₀ and A₁₇ is H;

or A₁₆ is H and A₁₇ is a group of formula:

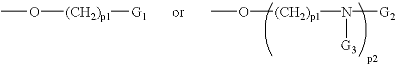

wherein:

G₁ is —CN, —OA₂₀, —SA₂₀, —N(H)A₂₀, —ON(H)A₂₀ or —C(═NH)N(H)A₂₀;

G₂ is H, —NHA₂₀, —C(═O)N(H)A₂₀, —C(═S)N(H)A₂₀ or —C(═NH)N(H)A₂₀,

each G₃ is, independently, H or an amino protecting group;

A₂₀ is H, a protecting group, substituted or unsubstituted C₁-C₁₀ alkyl, acetyl, benzyl, —(CH₂)ₚ₃N(H)G₃, a D or L α-amino acid, or a peptide derived from D, L or racemic α-amino acids;

each R₅ is carbonyl protecting group;

each p1 is from 2 to about 6;

p2 is from 1 to about 3; and

p3 is from 1 to about 4.

This invention also provides compounds having the structure:

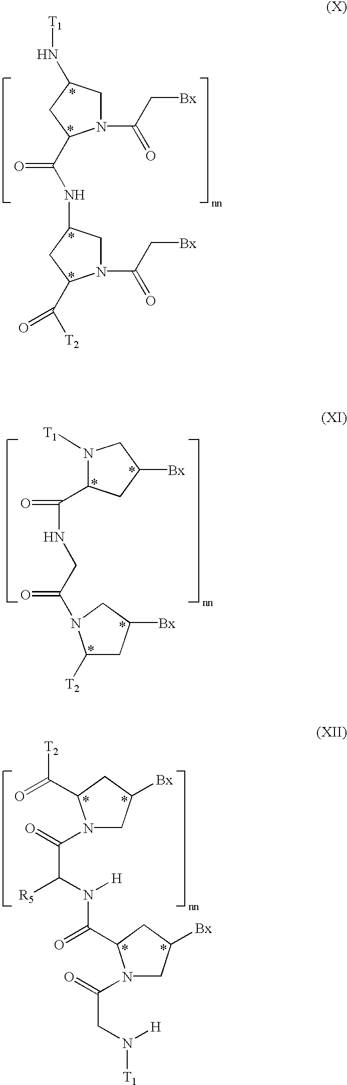

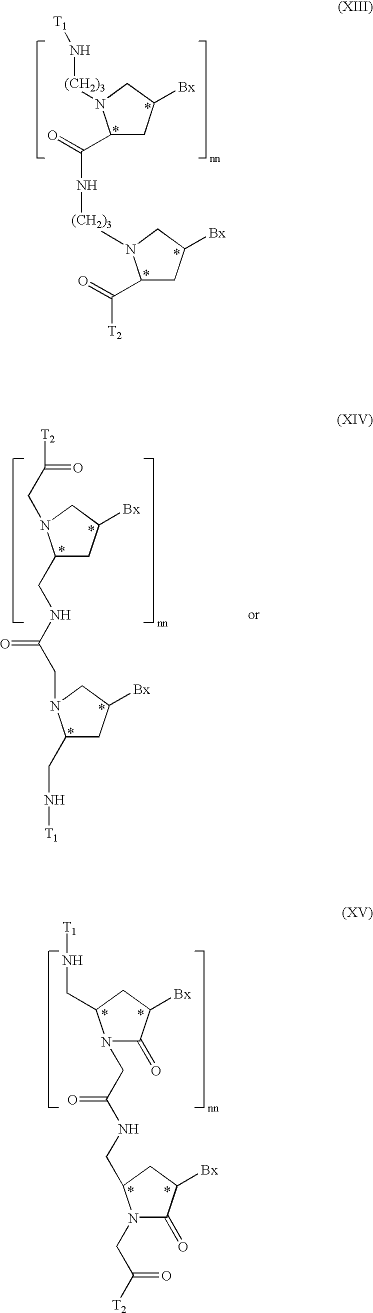

wherein:

T₁ is hydrogen, an amino protecting group, —C(O)R₅, substituted or unsubstituted C₁-C₁₀ alkyl, substituted or unsubstituted C₂-C₁₀ alkenyl, substituted or unsubstituted C₂-C₁₀ alkynyl, alkylsulfonyl, arylsulfonyl, a chemical functional group, a reporter group, a conjugate group, a D or L α-amino acid linked via the α-carboxyl group or optionally through the ω-carboxyl group when the amino acid is aspartic acid or glutamic acid or a peptide derived from D, L or mixed D and L amino acids linked through a carboxyl group, wherein the substituent groups are selected from hydroxyl, amino, alkoxy, carboxy, benzyl, phenyl, nitro, thiol, thioalkoxy, halogen, alkyl, aryl, alkenyl and alkynyl;

T₂ is —OH, —N(Z₁)Z₂, R₅, D or L α-amino acid linked via the α-amino group or optionally through the i-amino group when the amino acid is lysine or ornithine or a peptide derived from D, L or mixed D and L amino acids linked through an amino group, a chemical functional group, a reporter group or a conjugate group;

nn is from 2 to about 50;

each chiral ring carbon having an asterick (*) is prepared having R, S or mixed R and S configuration;

each Bx is, independently, an optionally protected heterocyclic base moiety wherein at least one of said heterocyclic base moieties has one of formulas II or III:

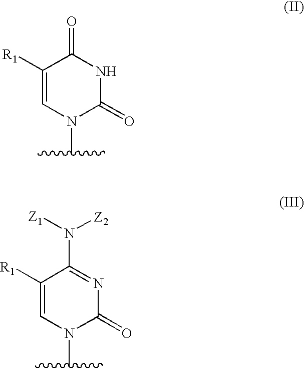

wherein:

R₁ is —CH₂—Q₁, —C≡C—Q₂, —CH₂—(CH₂)ₙ—Q₃, or —CH═CH—C(═O)—Q₄;

Q, is —N₃, —CN, —N(Z₁)Z₂, —N(Z₁)—(CH₂)ₙ—C(═NH₂)—N(H)—Z₃, —N(Z₁)—C(═J)—N(H)—Z₅, —L—(CH₂)ₙ—C(═O)Z₃, —L—(CH₂)ₙ—L—Z₃, —L—(CH₂),—N(H)Z₁, —L—(CH₂)ₙ—N(Z₁)—(CH₂)ₙ—N(H)Z₁, —L—(CH₂)ₙ—C(═NH)N(Z₁)Z₃ or —L—(CH₂)ₙ—N(Z₁)—C(═J)—N(H)Z₃;

Q₂ is H, C₁-C₆ alkyl, —C(═O)—N(H)Z₁, —C(═O)—O—CH₂—CH₃, C(═O)—O—benzyl, —C(═O)—Z₄, —CH₂—O—Q₆, —CH₂—C(—NH)—N(H)—Z₃, —CH₂—N(H)—Z₂, —CH₂—N(H)—C(═O)—CF₃, —CH₂—N(H)Z₁, —CH₂—N(H)—C(═NH)—N(H)—Z₃, —CH₂—O—N(H)—C(═O)—(CH₂)ₙ—Q₅, —C(═O)—N(H)—(CH₂)ₙ—Q₅ or —CH₂—N(H)—C(═O)—(CH₂)ₙ—Q₅;

Q₃ is hydrogen, —O—C₁-C₆ alkyl, —N(H)—Z₁, —N(H)—Z₂, —N(H)—C(═O)—CF₃, N(H)—C(═NH)—N(H)Z₁, —O—Q₆, —N(H)—C(═O)—(CH₂),—Q₅, —O—N(H)—C(═O)—(CH₂)ₙ—Q₅, —C(═O)—N(H)—(CH₂)ₙ—Q₅ or —C(═O)—Q₇;

Q₄ is is Z₄, —L—Z₃, —N(H)Z₃, —C(═NH)—N(H)Z₃, —N(H)—C(═J) N(H)Z₃, N(Z₁)—(CH₂)ₙ—N(H)Z₃, —C(═O)—N(H)—(CH₂)ₙ—C(═NH)—N(H)Z₃, —C(═O)—N(H)—(CH₂)ₙ—N(H)—C(═J)—N(H)Z₃ or —C(═O)—N(H)—(CH₂)ₙ—N(Z₁)—(CH₂)ₙ—N(H)Z₁;

Q₅ is —L—Z₃, —N(H)Z₃, —C(═NH)—N(H)Z₃, —N(H)—C(═J) N(H)Z₃ or N(Z₁)—(CH₂)ₙ—N(H)Z₃;

Q₆ is hydrogen, —N(H)Z₁, —N(H)Z₂, benzyl, benzoyl, —C(═O)—(CH₂)ₙ—H or phthalimido;

Q₇ is —OH, —O—C₁-C₆ alkyl, —O-benzyl, —Z₄, —N(H)Z₁,

each L is O or S;

each J is O, S or NH;

each n is from 1 to 6;

Z₁ is hydrogen, C₁-C₆ alkyl, or an amino protecting group;

Z₂ is hydrogen, C₁-C₆ alkyl, an amino protecting group, —C(═O)—(CH₂)ₙ—J—Z₃, a D or L α-amino acid linked via the α-carboxyl group or optionally through the ω-carboxyl group when the amino acid is aspartic acid or glutamic acid or a peptide derived from D, L or mixed D and L amino acids linked through a carboxyl group;

Z₃ is hydrogen, an amino protecting group, —C₁-C₆ alkyl, —C(═O)—CH₃, benzyl, benzoyl, or —(CH₂),—N(H)Z₁;

Z₄ is a D or L α-amino acid linked via the α-amino group or optionally through the ω-amino group when the amino acid is lysine or ornithine or a peptide derived from D, L or mixed D and L amino acids linked through an amino group;

Z₅ is hydrogen, an amino protecting group or —C(═O)—(CH₂)ₙ—J—Z₃; and

each R₅ is a carbonyl protecting group.

Further compounds according to this invention include those having the structure:

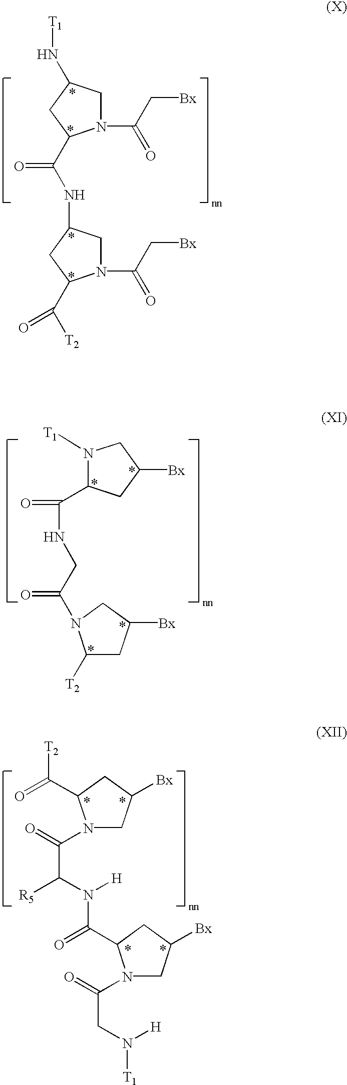

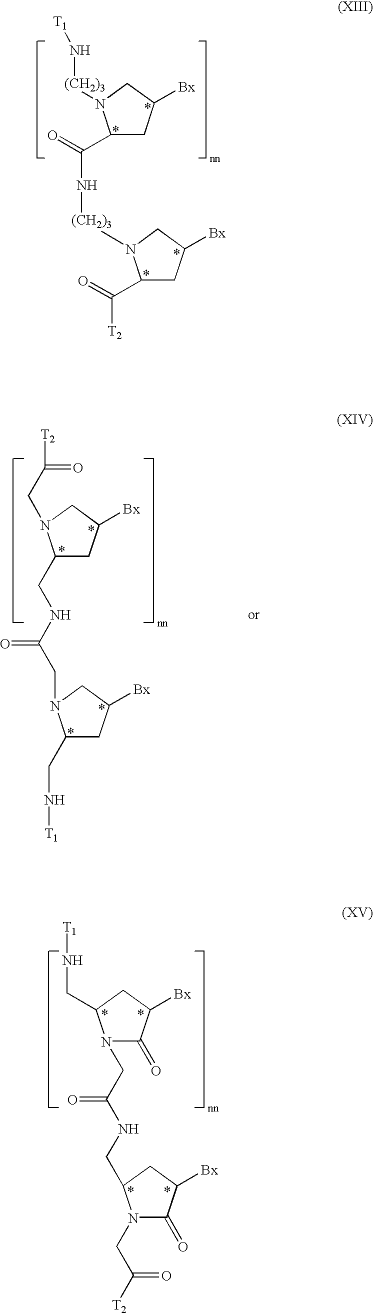

wherein:

T₁ is hydrogen, an amino protecting group, —C(O)R₅, substituted or unsubstituted C₁-C₁₀ alkyl, substituted or unsubstituted C₂-C₁₀ alkenyl, substituted or unsubstituted C₂-C₁₀ alkynyl, alkylsulfonyl, arylsulfonyl, a chemical functional group, a reporter group, a conjugate group, a D or L α-amino acid linked via the α-carboxyl group or optionally through the ω-carboxyl group when the amino acid is aspartic acid or glutamic acid or a peptide derived from D, L or mixed D and L amino acids linked through a carboxyl group, wherein the substituent groups are selected from hydroxyl, amino, alkoxy, carboxy, benzyl, phenyl, nitro, thiol, thioalkoxy, halogen, alkyl, aryl, alkenyl and alkynyl;

T₂ is —OH, —N(Z₁)Z₂, R₅, D or L α-amino acid linked via the α-amino group or optionally through the ω-amino group when the amino acid is lysine or ornithine or a peptide derived from D, L or mixed D and L amino acids linked through an amino group, a chemical functional group, a reporter group or a conjugate group;

nn is from 2 to about 50;

each chiral ring carbon having an asterick (*) is prepared having R, S or mixed R and S configuration;

each Bx is, independently, an optionally protected heterocyclic base moiety wherein at least one of said heterocyclic base moieties has one of formulas V or VI:

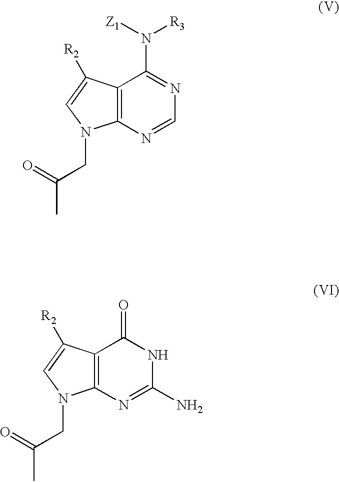

wherein:

R₂ is hydrogen and R₃ is Z₁, —C(═J)—N(H)Z₁, —C(═O)—(CH₂)ₙ—N(H)Z₁, —C(═O)—(CH₂)ₙ—L—Z₉, —(CH₂)ₙ—N(H)Z₁, —(CH₂)ₙ—N(Z₁)—(CH₂)ₙ—N(H)Z₁, —(CH₂)ₙ—N(H)—C(═J)—N(H)Z₃ or —(CH₂)ₙ—C(═NH)—N(H)Z₃;

or R₃ is hydrogen and R₂ is —C≡C—R₄ or —(CH₂)ₘ—R₄;

L is O or S;

J is O, S, or NH;

m is from 2 to 6;

each n is from 1 to 6;

R₄ is H, C₁-C₆ alkyl, —CH₂OH, —CH₂—O—Q₆, —CH₂—N(H)—C(═O)—CF₃, —CH₂—N(H)Z₁, —CH₂—N(H)Z₂, —C(═O)—Z₄, —C(═O)—N(H)—(CH₂)ₙ—Q₅, —CH₂—N(H)—C(═O)—(CH₂)ₙQ₅ or —CH₂—O—N(H)—C(═O)—(CH₂)ₙ—Q₅;

Q₅ is —L—Z₃, —N(H)Z₁, —C(═NH)—N(H)Z₃, —N(H)—C(═J) N(H)Z₃ or —N(Z₁)—(CH₂)ₙ—N(H)Z₁;

Q₆ is —N(H)Z₁, —N(H)Z₂, benzyl, benzoyl, —C(═O)—(CH₂)ₙ—CH₃ or phthalimido;

Z₁ is hydrogen, C₁-C₆ alkyl, or an amino protecting group;

Z₂ is hydrogen, C₁-C₆ alkyl, an amino protecting group, —C(═O)—(CH₂)ₙ—J—Z₃, a D or L α-amino acid linked via the α-carboxyl group or optionally through the ω-carboxyl group when the amino acid is aspartic acid or glutamic acid or a peptide derived from D, L or mixed D and L amino acids linked through a carboxyl group;

Z₃ is hydrogen, an amino protecting group, —C₁-C₆ alkyl, —C(═O)—CH₃, benzyl, benzoyl, or —(CH₂)ₙ—N(H)Z₁;

Z₄ is —OH, C₁-C₆ alkyl, benzyl, —N(H)Z₁, a D or L α-amino acid linked via the α-amino group or optionally through the ω-amino group when the amino acid is lysine or ornithing or a peptide derived from D, L or mixed D and L amino acids linked through an amino group;

Z₉ is hydrogen, —C₁-C₆ alkyl, —C(═O)—CH₃, benzyl or a D or L α-amino acid linked via the α-carboxyl group or optionally through the ω-carboxyl group when the amino acid is aspartic acid or glutamic acid or a peptide derived from D, L or mixed D and L amino acids linked through a carboxyl group; and

each R₅ is a carbonyl protecting group.

In other embodiments this invention provides compounds having the structure:

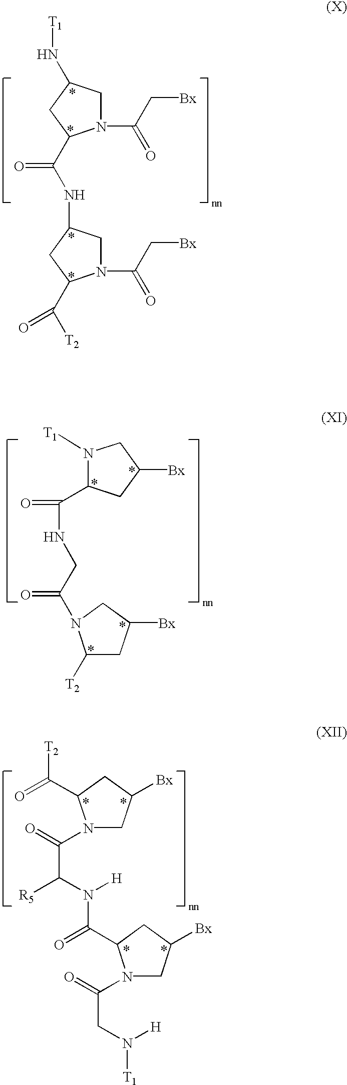

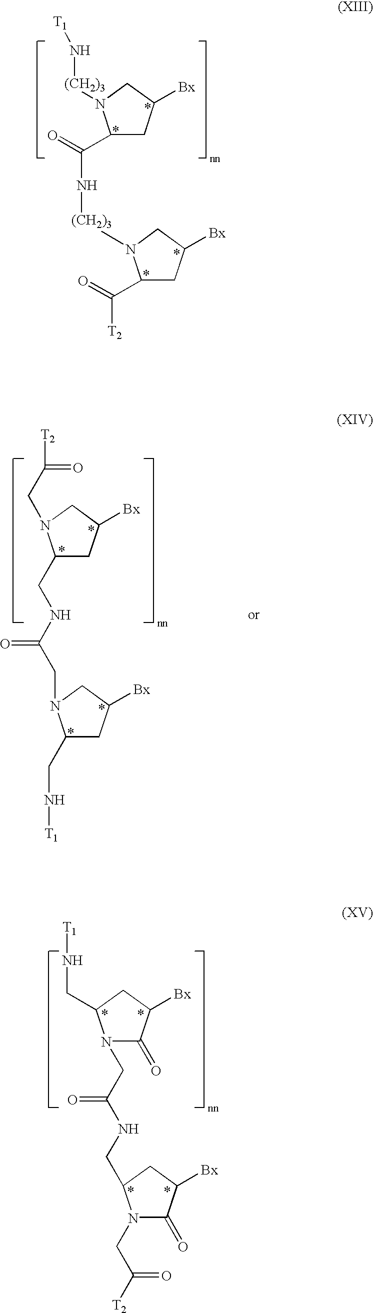

wherein:

T₁ is hydrogen, an amino protecting group, —C(O)R₅, substituted or unsubstituted C₁-C₁₀ alkyl, substituted or unsubstituted C₂-C₁₀ alkenyl, substituted or unsubstituted C₂-C₁₀ alkynyl, alkylsulfonyl, arylsulfonyl, a chemical functional group, a reporter group, a conjugate group, a D or L α-amino acid linked via the α-carboxyl group or optionally through the ω-carboxyl group when the amino acid is aspartic acid or glutamic acid or a peptide derived from D, L or mixed D and L amino acids linked through a carboxyl group, wherein the substituent groups are selected from hydroxyl, amino, alkoxy, carboxy, benzyl, phenyl, nitro, thiol, thioalkoxy, halogen, alkyl, aryl, alkenyl and alkynyl;

T₂ is —OH, —N(Z₁)Z₂, R₅, D or L α-amino acid linked via the α-amino group or optionally through the ω-amino group when the amino acid is lysine or ornithine or a peptide derived from D, L or mixed D and L amino acids linked through an amino group, a chemical functional group, a reporter group or a conjugate group;

nn is from 2 to about 50;

each chiral ring carbon having an asterick (*) is prepared having R, S or mixed R and S configuration;

each Bx is, independently, an optionally protected heterocyclic base moiety wherein at least one of said heterocyclic base moieties has formula VIII:

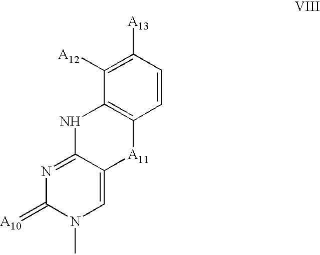

wherein

A₁₀ is O or S;

A₁₁ is CH₂, N—CH₃, O or S;

A₁₂ and A₁₃ is hydrogen or one of A₁₂ and A₁₃ is hydrogen and the other of A₁₂ and A₁₃ is a group of formula:

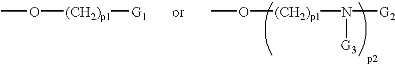

wherein:

G₁ is —CN, —OA₂₀, —SA₂₀, —N(H)A₂₀, —ON(H)A₂₀ or —C(═NH)N(H)A₂₀;

G₂ is H, —NHA₂₀, —C(═O)N(H)A₂₀, —C(═S)N(H)A₂₀ or —C(═NH)N(H)A₂₀, each G₃ is, independently, H or an amino protecting group;

A₂₀ is H, a protecting group, substituted or unsubstituted C₁-C₁₀ alkyl, acetyl, benzyl, —(CH₂)ₚ₃NH₂, —(CH₂)ₚ₃N(H)G₃, a D or L α-amino acid, or a peptide derived from D, L or racemic α-amino acids;

each R₅ is a carbonyl protecting group;

each p1 is, independently, from 2 to about 6;

p2 is from 1 to about 3; and

p3 is from 1 to about 4.

Other representative compounds of the present invention are shown below in Table 3:

TABLE 3

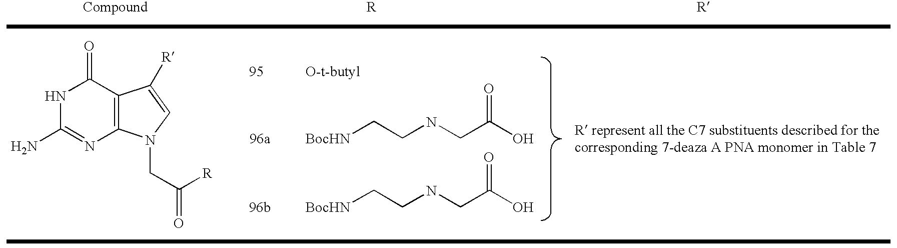

Still other representative compounds of the present invention are shown below in Table 4:

TABLE 4

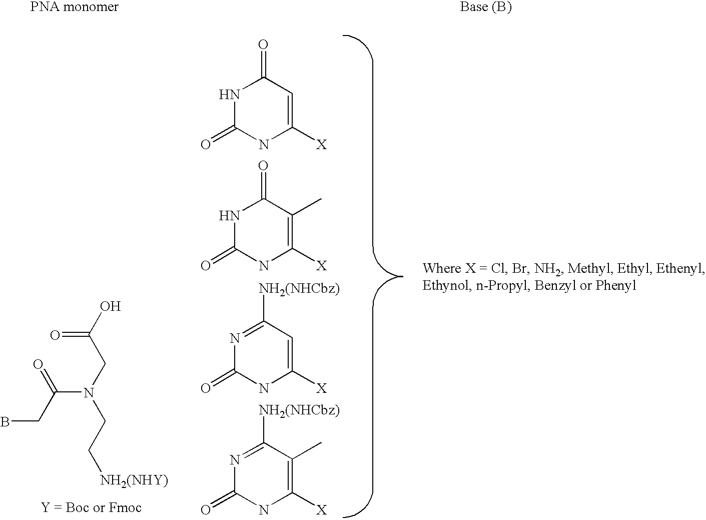

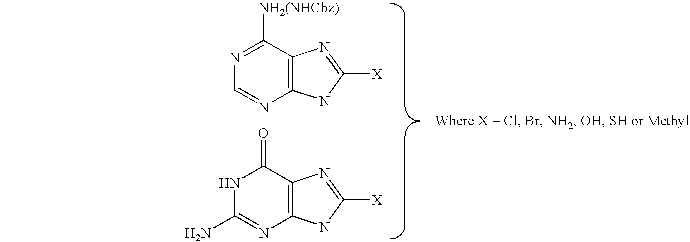

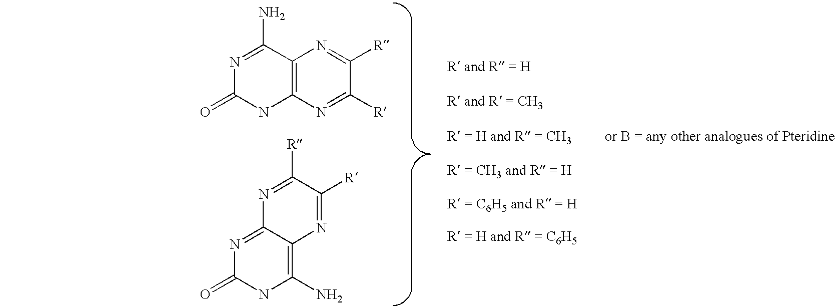

### BRIEF DESCRIPTION OF THE DRAWINGS

The numerous features and advantages of the present invention may be better understood by those skilled in the art by reference to the accompanying detailed description and the following drawings, in which:

FIG. 1 shows a representative synthesis of tricyclic compound 6a

FIG. 2 shows preparation of PNA oligomer 11.

FIG. 3 shows a representative synthesis of tricyclic compound 15.

FIG. 4 shows a representative synthesis of tricyclic compound 19.

FIG. 5 shows a representative synthesis of tricyclic compound 22b.

FIG. 6 shows a representative synthesis of tricyclic compounds 25a, 25b, 27a, and 27b.

FIG. 7 shows a representative synthesis of tricyclic compounds 34a, 34b, 35a, 35b, 36a, and 36b.

FIG. 8 shows a representative synthesis of tricyclic compounds 37a, 37b, 38a, and 38b.

FIG. 9 shows preparation of PNA oligomer 11a.

FIG. 10 shows a representative synthesis of monocyclic compounds 49a and 49b.

FIG. 11 shows a representative synthesis of monocyclic compounds 52a, 52b, 55a, and 55b.

FIG. 12 shows a representative synthesis of monocyclic compounds 63a and 63b.

FIG. 13 shows a representative synthesis of monocyclic compounds 65a, 65b, 67a, 67b, b 69a, and 69b.

FIG. 14 shows a representative synthesis of monocyclic compounds 73a and 73b.

FIG. 15 shows a representative synthesis of monocyclic compounds 78a and 78b.

FIG. 16 shows a representative synthesis of dicyclic compounds 84a, 84b, and 87.

FIG. 17 shows a representative synthesis of dicyclic compounds 90a, 90b, 93a, and 94b.

FIG. 18 shows a representative synthesis of tricyclic compound 37c.

### DETAILED DESCRIPTION OF THE INVENTION

In accordance with the present invention, peptide nucleic acids are provided. In preferred embodiments, these compounds exhibit enhanced cellular uptake and distribution. The peptide nucleic acids (PNAs) of the present invention are assembled from a plurality of naturally-occuring or non-naturally-occuring nucleobases that are attached to polyamide backbones by a suitable linker. Non-naturally occuring nucleobases include modified monocyclic bases, bicyclic bases, and tricyclic bases. Such PNAs may be prepared by solid state synthesis or by other means known to those skilled in the art.

“PNA compounds” or “PNA” refers to peptide nucleic acids that are artificial biopolymers, i.e., nucleic acid mimics, wherein the DNA sugar phosphate backbone of an oligonucleotide is replaced by a peptide backbone or psudopeptide backbone, PNA include amide backbones, e.g. an aminoethylglycine backbone, bound directly or indirectly to aza nitrogen atoms of the amide portion of the backbone. Naturally-occurring or non-naturally-occurring nucleobases are bound directly or indirectly to aza nitrogen atoms of the amide portion of the backbone. Representative United States patents that teach the preparation of PNA compounds include, but are not limited to, U.S. Pat. Nos.: 5,539,082; 5,714,331; and 5,719,262, each of which is herein incorporated by reference. Further teaching of PNA compounds can be found in Nielsen et al., Science, 1991, 254, 1497-1500.

“Oligonucleotide” refers to polynucleotides, formed by joining naturally-occuring, non-naturally-occuring bases, or furanosyl groups. Thus, this term effectively refers to naturally occurring species or synthetic species formed from naturally occurring subunits or their close homologs. The term “oligonucleotide” or “oligomer” may also refer to moieties which have portions similar to naturally occurring oligonucleotides but which have non-naturally occurring portions. Thus, oligonucleotides may have altered sugars, altered base moieties, or altered inter-sugar linkages. Exemplary among these are the phosphorothioate and other sulfur-containing species which are known for use in the art.

Oligonucleotides may also include species which include at least some modified base forms. Thus, purines and pyrimidines other than those normally found in nature may be so employed. Suitable bases include, but are not limited to those described in U.S. Pat. No. 3,687,808.

Oligonucleotides may also comprise other modifications that are best described as being functionally interchangeable with yet structurally distinct from natural oligonucleotides. All such oligonucleotides are comprehended by this invention so long as they effectively function as subunits in the oligonucleotide. “Nucleoside” refers to a sugar and a base that are joined together.

A “reporter group” is any structure known to those skilled in the art that can be added to an oligonucleotide or PNA so that the oligonucleotide or PNA can be detected. For example, reporter groups include radioisotopes; enzymes; flourescent structures; chromogens (fluorescent or luminescent groups and dyes); enzymes; NMR-active groups or metal particles; haptens, e.g. digoxigenin, or biotin and derivatives thereof; photoactivatable crosslinking groups, e.g. an azido or an azirine group; metal chelates which can be detected by electrochemoluminescence; an intercalator which can intercalate into a PNA-nucleic acid hybrid and in this way enable it to be detected, where appropriate, e.g., thiazole orange, ethidium bromide and propidium iodide; pharmaceutically active groups; or a group which is able to improve the pharmacodynamic or pharmacokinetic properties. Reporter groups of different types are described in WO 94/068 15, U.S. patent application Ser. No. 07/555,323 filed Jul. 19, 1990, which are herein incorporated by reference in their entirety. Reporter groups may also include optional linking groups.

PNAs exhibit significant advantages over natural nucleic acids, including for example, ease of synthesis compared to synthesis of natural nucleic acids, very good stability to cellular nucleases and proteases, and the capability of hybridizing with complimentary DNA with high affinity.

PNA binds both DNA and RNA to form PNA/DNA or PNA/RNA duplexes. The resulting PNA/DNA or PNA/RNA duplexes are bound with greater affinity than corresponding DNA/DNA or DNA/RNA duplexes as determined by Tm's. This high thermal stability might be attributed to the lack of charge repulsion due to the neutral backbone in PNA. The neutral backbone of the PNA also results in the Tm's of PNA/DNA(RNA) duplex being practically independent of the salt concentration. Thus the PNA/DNA duplex interaction offers a further advantage over DNA/DNA duplex interactions which are highly dependent on ionic strength. Homopyrimidine PNAs have been shown to bind complementary DNA or RNA forming (PNA)2/DNA(RNA) triplexes of high thermal stability (see, e.g., Egholm, et al., Science, 1991, 254, 1497; Egholm, et al., J. Am. Chem. Soc., 1992, 114, 1895; Egholm, et al., J. Am. Chem. Soc., 1992, 114, 9677).

The binding of a PNA strand to a DNA or RNA strand can occur in one of two orientations. The orientation is said to be anti-parallel when the DNA or RNA strand in a 5′ to 3′ orientation binds to the complementary PNA strand such that the carboxyl end of the PNA is directed towards the 5′ end of the DNA or RNA and amino end of the PNA is directed towards the 3′ end of the DNA or RNA. In the parallel orientation the carboxyl end and amino end of the PNA are just the reverse with respect to the 5′-3′ direction of the DNA or RNA.

PNAs bind to both single stranded DNA and double stranded DNA. As noted above, in binding to double stranded DNA it has been observed that two strands of PNA can bind to the DNA. While PNA/DNA duplexes are stable in the antiparallel configuration, it was previously believed that the parallel orientation is preferred for (PNA)₂/DNA triplexes.

The binding of two single stranded pyrimidine PNAs to a double stranded DNA has been shown to take place via strand displacement, rather than conventional triple helix formation as observed with triplexing oligonucleotides. When PNAs strand invade double stranded DNA, one strand of the DNA appears to be displaced and forms a loop on the side of the PNA₂/DNA complex area. The other strand of the DNA is locked up in the (PNA)₂/DNA triplex structure. Since the loop area (alternately referenced as a D loop) is single stranded, it is susceptible to cleavage by enzymes that can cleave single stranded DNA.

A further advantage of PNA compared to oligonucleotides is that their polyamide backbone (having appropriate nucleobases or other side chain groups attached thereto) is not recognized by either nucleases or proteases and is not cleaved. As a result PNAs are resistant to degradation by enzymes, unlike DNA and peptides.

In accordance with this invention, it has been found that the most stable triplexes that are formed between two single stranded PNAs or a bis PNA and a DNA or RNA target strand are triplexes wherein the Watson/Crick base pairing strand is in an anti- parallel orientation relative to the target strand and the Hoogsteen base pairing strand is in a parallel orientation relative to the target strand. As so orientated to the target strand, the two PNA strands are therefore anti-parallel to each other.

Bis PNAs have shown improved binding affinity, thermal stability, and specificity over single-stranded PNAs. Using dsDNA as a target it has been shown that the preferred orientation is with the first PNA strand of the bis PNA parallel to the target, i.e. the target DNA strand of the duplex is referenced in a 5′ to 3′ direction and the first PNA is complementary in an N to C direction, and the second PNA strand of the bis PNA is antiparallel to the target, i.e. it is complementary to the DNA strand (again referenced in a 5′ to 3′ direction) in a C to N direction. Thus the linking segment connects the PNA strands in opposite orientation to each other, i.e. from a common reference point, one strand is lined up in a N to C direction and the other is lined up in a C to N direction.

Although we do not wish to be bound by theory it is believed that the antiparallel strand of the bis PNA binds the DNA target thereby displacing the other DNA strand via strand invasion. This binding is of a Watson/Crick nature. The second PNA strand of the bis PNA, the parallel strand, now binds the DNA using Hoogsteen type hydrogen bonding. It has been shown using the component single stranded PNAs and comparing them separately and as a mixture to the bis PNA that the bis PNA has a faster “on rate” e.g. it binds faster to the target. This faster on rate is attributed to the enforced close proximity of the second strand in the bis PNA.

We have also studied the effect of pH on the Tm of bis PNA bound to dsDNA as compared to the same bis PNA with the cytosines replaced with pseudo isocytosines. It has been observed in previous studies that there is a pronounced dependence on pH for binding of PNA to dsDNA. The decrease in Tm with higher pH shows that Hoogsteen binding in a (PNA)₂/DNA complex is pH dependent. Normal Hoogsteen binding requires that the cytosines be protonated. This makes the Hoogsteen strand binding pH dependent. We have found that replacement of one or more of the cytosine nucleobases in a Hoogsteen strand with pseudo isocytosine and other like nucleobases removes this dependence.

To demonstrate this effect, in two bis PNAs of the invention, one was synthesized such that the cytosines nucleobases in the parallel strand were replaced with pseudo isocytosines and the other was synthesized such that the cytosines in the antiparallel strand were replaced with pseudo isocytosines. The bis PNA with the pseudo isocytosines in the parallel strand showed almost no dependence on pH indicating that the parallel strand is involved with Hoogsteen binding.

The replacement of cytosine by pseudo isocytosine or other like C-pyrimidine nucleobases is effected in a straight forward manner as per certain of the examples set forth below. This is in direct contrast with replacement of cytosine with pseudo isocytosine or other C-pyrimidines in nucleosides. In nucleosides, an anomeric specific carbon-carbon bond must be formed in synthesizing the C-nucleoside. Since there are no anomeric (sugar) carbon atoms in peptide nucleic acids, such constraints need not be considered.

The triple helix principle is used in the art for sequence-specific recognition of dsDNA. Triple helix formation utilizes recognition of homopurine-homopyrimidine sequences. A strand displacement complex with triple helix formation is believed to be superior to simple triple helix recognition in that strand displacement complexes are very stable at physiological conditions, that is, neutral pH, ambient (20-40 degrees Centigrade) temperature and medium (100-150 mM) ionic strength.

Sequence-specific recognition of ssDNA by base complementary hybridization can likewise be exploited to target specific genes and viruses. In this case, the target sequence is contained in the mRNA such that binding of the drug to the target hinders the action of ribosomes and, consequently, translation of the mRNA into protein. The bis PNAs of the invention appear to be superior to prior reagents in that they have significantly higher affinity for complementary ssDNA. Also, they can be synthesized such that they possess no charge and are water soluble, which should facilitate cellular uptake, and they contain amides of non-biological amino acids, which should make them biostable and resistant to enzymatic degradation by, for example, proteases.

The PNA backbones of the present invention can be modified. For example, PNAs having modified backbones are described in U.S. Pat. No. 5,719,262, issued Feb. 17, 1998, hereby incorporated by reference in its entirety. Further PNA backbone sustitutions at the glycinyl methylene group are disclosed in U.S. Pat. No. 6,107,470, issued Aug. 22, 2000, hereby incorporated by reference in its entirety.

Other modifications of the backbone (including various combinations of substitution at the glycinyl methylene, varying the chain length of the aminoethyl group and or the glycinyl group, and the tethering group) can be included in the compounds of the present invention. For example, PNAs having these modifications are disclosed in U.S. Pat. No. 5,641,625, issued Jun. 24, 1997, hereby incorporated by reference in its entirety. Further backbone modifications and substitutions are disclosed in U.S. Pat. No. 5,773,571, issued Jun. 30, 1998, hereby incorporated by reference in its entirety.

Other representative compounds of the present invention include those shown below in Table 5:

TABLE 5

| Compound | B(“G-clamp” base) |
|---|---|
| 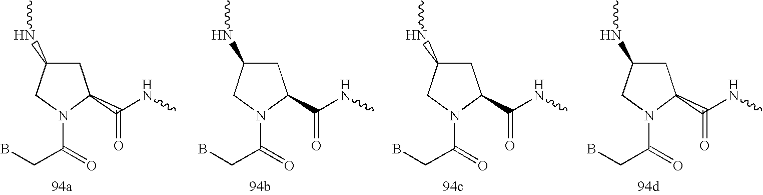 | 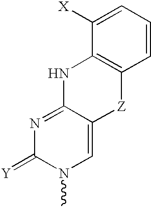 |
| 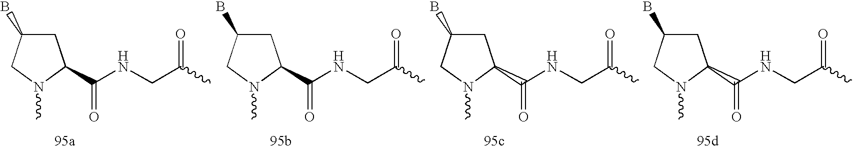 | Where, X = is independently selected from all the substituted described in Table 1 Y = O or S, and Z = O or S |
| 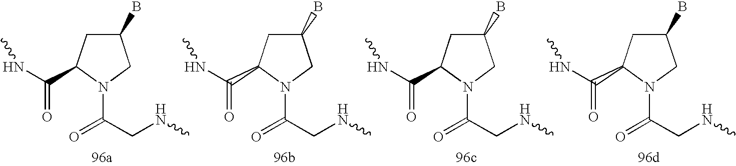 | |
| 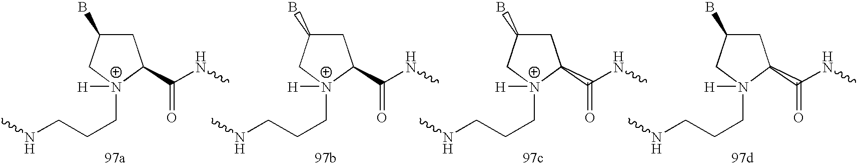 | |
| 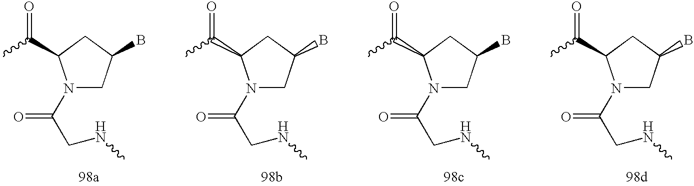 | |
| 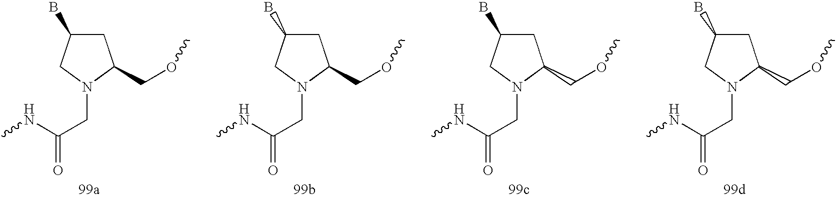 | |
| 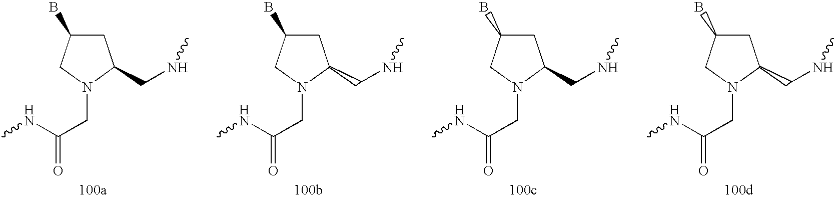 | |
| 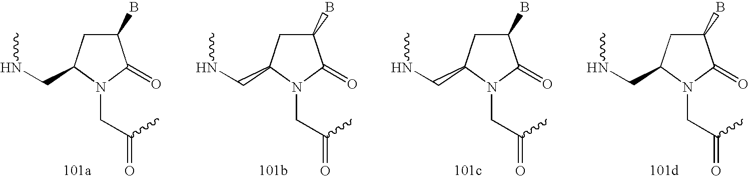 | |

The preparation of PNA monomers and oligomers having a cyclic structure incorporated into the backbone wherein the cyclic structure could give chirality to two of the carbon atoms of the backbone is disclosed in U.S. Pat. Nos. 5,977,296, issued Nov. 2, 1999, and U.S. Pat. No. 6,201,103, issued Mar. 13, 2001, hereby incorporated by reference in their entirety.

In some embodiments PNAs of the present invention include one or more amino acid moieties within their structure. These amino acids may be naturally-occurring or non-naturally-occurring. Naturally-occurring amino acids include a-amino acids where the chiral center has a D-configuration. Such naturally-occurring amino-acids may be either essential or non-essential amino acids. Non-naturally-occurring amino acids used in the PNAs of the present invention include α-amino acids with chiral centers bearing an L-configuration. Non-naturally-occurring amino acids also include amino acids bearing unusual side chains that do not exist in nature and are prepared synthetically, such as halo- and cyano-substituted benzyl, tetrahydroisoquinolylmethyl, cyclohexylmethyl, and pyridylmethyl. Other synthetic amino-acids include b-amino acids.

The amino acids may be introduced into PNAs either as part of the monomer used or at the terminal ends of the PNA. Any of the abovementioned amino acids could be incorporated into the monomeric building blocks used in PNA synthesis. Amino acids may also be attached at the C-terminus of PNAs such that the terminal R^{h}—CO-group represents an amino acyl group derived from any naturally- or non-naturally-occurring amino acid, a- or b- amino acid, and with a D- or L-configuration at the a-chiral center. Amino acids may also be incorporated at the N-terminal end of a PNA.

Preferrably, the present PNAs have nn from about 8 to about 30. More preferably, nn is from about 15 to about 25.

In other preferred embodiments, each carbonyl protecting group is, independently, substituted or unsubstituted C₁-C₁₀ alkyl, trifluoromethyl, cyanoethyloxy, methoxy, ethoxy, t-butoxy, allyloxy, 9-fluorenylmethoxy, 2-(trimethylsilyl)-ethoxy, 2,2,2-trichloroethoxy, benzyloxy, butyryl, iso-butyryl, phenyl or aryl.

In still other preferred embodiments, the conjugate group is a contrast reagent, a cleaving agent, a cell targeting agent, polyethylene glycol, cholesterol, phospholipid, biotin, phenanthroline, phenazine, phenanthridine, anthraquinone, acridine, fluorescein, rhodamine, coumarin, pyrene, retinal or a cyanine dye.

Preferably PNAs of the present invention include from about 6 to about 50 nucleobases. More preferrably PNAs include from about 12 to about 20 nucleobases.

The PNAs of the present invention include compounds of Formula I wherein nucleobase Bx has the Formula II or III. These nucleobases are attached to the PNA backbone by a suitable linker.

In preferred compounds of Formula I wherein nucleobase Bx has the Formula II or III, R₁ is —CH₂—Q₁ and Q₁ is —N₃, —CN, —N(Z₁)Z₂, —N(Z₁)—(CH₂)ₙ—C(═NH)—N(H)—Z₃, —N(Z₁)—C(═J)—N(H)—Z₅, —L—(CH₂)ₙ—C(═O)Z₃, —L—(CH₂)ₙ—L—Z₃, —L—(CH₂)ₙ—N(H)Z₁, —L—(CH₂)ₙ—N(Z₁)—(CH₂)ₙ—N(H)Z₁, —L—(CH₂)ₙ—C(═NH)N(Z₁)Z₃ or —L—(CH₂)ₙ—N(Z₁)—C(═J)—N(H)Z₃.

In other preferred embodiments, R₁ is —CH₂—(CH₂)ₙ—Q₃. Q₃ is hydrogen, —O—CH₃, —O—CH₂CH₃, —N(H)—Z₁, —N(H)—Z₂, —N(H)—C(═O)—CF₃ or —N(H)—C(═NH)—N(H)Z₁. Preferrably each Q₃ is —N(H)—C(═O)—(CH₂)ₙ—Q₅, —O—N(H)—C(═O)—(CH₂)ₙ—Q₅ or —C(═O)—N(H)—(CH₂)ₙ—Q₅ and Q₅is —N(H)Z₃, —C(═NH)—N(H)Z₃ or —N(H)—C(═J) N(H)Z₃. More preferrably Q₃ is —O—Q₆ and Q₆ is hydrogen, —N(H)Z₁or —N(H)Z₂.

In still other preferred compounds of Formula I wherein nucleobase Bx has the Formula II or III, R₁ is —C≡C—Q₂ and, Q₂ is H, methyl, ethyl, —C(═O)—N(H)Z₁, —CH₂—N(H)—Z₂ or —CH₂—N(H)—C(═NH)—N(H)—Z₅.

In still other preferred compounds of Formula I wherein nucleobase Bx has the Formula II or III, R₁ is —CH₂—(CH₂)ₙ—Q₃. Q₃ is hydrogen, —O—CH₃, —O—CH₂CH₃, —N(H)—Z₁, —N(H)—Z₂, —N(H)—C(═O)—CF₃ or —N(H)—C(═NH)—N(H)Z₁. Preferrably each Q₃ is —N(H)—C(═O)—(CH₂)ₙ—Q₅, —O—N(H)—C(═O)—(CH₂)ₙ—Q₅ or —C(═O)—N(H)—(CH₂)ₙ—Q₅ and Q₅ is —N(H)Z₃, —C(═NH)—N(H)Z₃ or —N(H)—C(═J) N(H)Z₃. More preferrably Q₃ is —O—Q₆ and Q₆ is hydrogen, —N(H)Z₁or —N(H)Z₂.

In still other preferred compounds of Formula I wherein nucleobase Bx has the Formula II or III, each R₁ is —CH═CH—C(═O)—Q₄ and Q₄ is —OH, —N(H)Z₃, —C₁-C₆ alkyl, —O—C₁-C₆ alkyl, —O-benzyl or —N(H)—(CH₂)ₙ—Q₅. Q₄ is —N(H)Z₃ and Z₃ is Hydrogen or C₁-C₆ alkyl.

In preferred compounds of Formula I wherein nucleobase Bx has the Formula II or III, Z₁, Z₂, Z₃, Z₄ and Z₅ are each independently hydrogen, methyl or an amino protecting group. Each n is independently from 1 to about 3.

In still other preferred compounds of Formula I wherein nucleobase Bx has the Formula II or III, T₁ is hydrogen, an amino protecting group, a reporter group or a D or L amino acid or a peptide and the D or L amino acid is lysine or glutamic acid. T₂ is —OH, —N(Z₁)Z₂, R₅ or a D or L amino acid or a peptide.

Preferrably in compounds of Formula I wherein nucleobase Bx has the Formula II or III, each Bx is independently selected from the group consisting of a radical of formula II, formula III, adeninyl, guaninyl, thyminyl, cytosinyl, uracilyl, 5-methylcytosinyl (5-me-C), 5-hydroxymethyl cytosinyl, xanthinyl, hypoxanthinyl, 2-aminoadeninyl, alkyl derivatives of adeninyl and guaninyl, 2-thiouracilyl, 2-thiothyminyl, 2-thiocytosinyl, 5-halouracilyl, 5-halocytosinyl, 5-propynyl uracilyl, 5-propynyl cytosinyl, 6-azo uracilyl, 6-azo cytosinyl, 6-azo thyminyl, 5-uracilyl (pseudouracil), 4-thiouracilyl, 8-substituted adeninyls and guaninyls, 5-substituted uracilyls and cytosinyls, 7-methylguaninyl, 7-methyladeninyl, 8-azaguaninyl, 8-azaadeninyl, 7-deazaguaninyl, 7-deazaadeninyl, 3-deazaguaninyl and 3-deazaadeninyl.

The PNAs of the present invention include compounds of Formula I wherein nucleobase Bx has the Formula V or VI. These nucleobases are attached to the PNA backbone by a suitable linker.

In preferred compounds of Formula I wherein nucleobase Bx has the Formula V or VI, R₂ is hydrogen and R₃ is Z₁, —C(═J)—N(H)Z₁or —(CH₂)ₙ—C(═NH)—N(H)Z₃.

In another preferred compounds of Formula I wherein nucleobase Bx has the Formula V or VI, R₂ is —C(═O)—(CH₂)ₙ—L—Z₉ and Z₉ is hydrogen, —C₁-C₃ alkyl or —C(═O)—CH₃.

In still other preferred compounds of Formula I wherein nucleobase Bx has the Formula V or VI, R₃ is hydrogen and R₂ is —C≡C—R₄ or —(CH₂)ₘ—R₄. R₄ is H, C₁-C₃ alkyl, —CH₂OH, —CH₂—O—Q₆, —CH₂—N(H)Z₂ or —C(═O)—Z₄. In another aspect of this embodiment, R₄ is —C(═O)—Z₄, —C(═O)—N(H)—(CH₂)ₙ—Q₅, —CH₂—N(H)—C(═O)—(CH₂)ₙQ₅ or —CH₂—O—N(H)—C(═O)—(CH₂)ₙ—Q₅ and Q₅is —N(H)Z₁or —C(═NH)—N(H)Z₃. In yet another aspect of this embodiment, R₄ is —CH₂—O—Q₆ and Q₆ is —N(H)Z₂, —C(═O)—(CH₂)ₙ—CH₃ or phthalimido. In still another aspect of this embodiment, R₄ is —C(═O)—Z₄ and Z₄ is —OH, C₁-C₃ alkyl, benzyl or —N(H)Z₁.

In still other preferred compounds of Formula I wherein nucleobase Bx has the Formula V or VI, R₃ is —(CH₂)ₙ—N(H)—C(═J)—N(H)Z₃ or —(CH₂)ₙ—C(═NH)—N(H)Z₃ and Z₃ is hydrogen, an amino protecting group, —C₁-C₃ alkyl or —C(═O)—CH₃.

In still other preferred compounds of Formula I wherein nucleobase Bx has the Formula V or VI, T₁ is hydrogen, an amino protecting group, a reporter group, a D or L amino acid or a peptide. More preferably, D or L amino acid is lysine or glutamic acid. T₂ is —N(Z₁)Z₂ and Z₂ is hydrogen, C₁-C₃ alkyl, an amino protecting group. In another preferred embodiment, T₂ is —OH, —(Z₁)Z₂, R₅, a D or L amino acid or a peptide.

In still other preferred compounds of Formula I wherein nucleobase Bx has the Formula V or VI, each Bx is independently selected from the group consisting of a radical of formula V, formula VI, adeninyl, guaninyl, thyminyl, cytosinyl, uracilyl, 5-methylcytosinyl (5-me-C), 5-hydroxymethyl cytosinyl, xanthinyl, hypoxanthinyl, 2-aminoadeninyl, alkyl derivatives of adeninyl and guaninyl, 2-thiouracilyl, 2-thiothyminyl, 2-thiocytosinyl, 5-halouracilyl, 5-halocytosinyl, 5-propynyl uracilyl, 5-propynyl cytosinyl, 6-azo uracilyl, 6-azo cytosinyl, 6-azo thyminyl, 5-uracilyl (pseudouracil), 4-thiouracilyl, 8-substituted adeninyls and guaninyls, 5-substituted uracilyls and cytosinyls, 7-methylguaninyl, 7-methyladeninyl, 8-azaguaninyl, 8-azaadeninyl, 7-deazaguaninyl, 7-deazaadeninyl, 3-deazaguaninyl and 3-deazaadeninyl.

The PNAs of the present invention include compounds of Formula I wherein nucleobase Bx has the Formula VIII. These nucleobases are attached to the PNA backbone by a suitable linker.

In preferred compounds of Formula I wherein nucleobase Bx has the Formula VIII, A₁₃ is H; A₁₂ is —O—(CH₂)₂—N(H)G₄, —O—(CH₂)₂—ON(H)G₄ or —O—(CH₂)₂—C(═NH)N(H)G₄, —O—(CH₂)₃—C(═NH)N(H)G₄, —O—(CH₂)₂—C(═O)N(H)G₄, —O—(CH₂)₂—C(═S)N(H)G₄ or —O—(CH₂)₂—N(H)C(═NH)N(H)G₄; and G₄ is hydrogen, an amino protecting group or C₁-C₁₀ alkyl. Preferably, A₁₀ is S and A₁₁ is O.

In other preferred compounds of Formula I wherein nucleobase Bx has the Formula VIII, T₁ is hydrogen, an amino protecting group, a reporter group, a D or L amino acid or a peptide. Preferably, D or L amino acid is lysine or glutamic acid.

In still other preferred compounds of Formula I wherein nucleobase Bx has the Formula VIII, T₂ is —OH, —(Z₁)Z₂, R₅, a D or L amino acid or a peptide.

In still other preferred compounds of Formula I wherein nucleobase Bx has the Formula VIII, each Bx is independently selected from the group consisting of a radical of formula VIII, adeninyl, guaninyl, thyminyl, cytosinyl, uracilyl, 5-methylcytosinyl (5-me-C), 5-hydroxymethyl cytosinyl, xanthinyl, hypoxanthinyl, 2-aminoadeninyl, alkyl derivatives of adeninyl and guaninyl, 2-thiouracilyl, 2-thiothyminyl, 2-thiocytosinyl, 5-halouracilyl, 5-halocytosinyl, 5-propynyl uracilyl, 5-propynyl cytosinyl, 6-azo uracilyl, 6-azo cytosinyl, 6-azo thyminyl, 5-uracilyl (pseudouracil), 4-thiouracilyl, 8-substituted adeninyls and guaninyls, 5-substituted uracilyls and cytosinyls, 7-methylguaninyl, 7-methyladeninyl, 8-azaguaninyl, 8-azaadeninyl, 7-deazaguaninyl, 7-deazaadeninyl, 3-deazaguaninyl and 3-deazaadeninyl.

The PNAs of the present invention include compounds of Formula I wherein nucleobase Bx has the Formula XVI. These nucleobases are attached to the PNA backbone by a suitable linker.

In preferred compounds of Formula I wherein nucleobase Bx has the Formula XVI, A₁₆ is H; A₁₇ is —O—(CH₂)₂—N(H)G₄, —O—(CH₂)₂—ON(H)G₄ or —O—(CH₂)₂—C(═NH)N(H)G₄, —O—(CH₂)₃—C(═NH)N(H)G₄, —O—(CH₂)₂—C(═O)N(H)G₄, —O—(CH₂)₂—C(═S)N(H)G₄ or —O—(CH₂)₂—N(H)C(═NH)N(H)G₄; and G₄ is hydrogen, an amino protecting group or C₁-C₁₀ alkyl. In one preferred embodiment A₁₅ is S. In another preferred embodiment A₁₅ is O.

In preferred compounds of Formula I wherein nucleobase Bx has the Formula XVI, n is from about 8 to about 30. More preferably, n is from about 15 to about 25.

In preferred compounds of Formula I wherein nucleobase Bx has the Formula XVI, T₁ is hydrogen, an amino protecting group, a reporter group, a D or L amino acid or a peptide. More preferrably, D or L amino acid is lysine or glutamic acid.

In preferred compounds of Formula I wherein nucleobase Bx has the Formula XVI, T₂ is —OH, —(Z₁)Z₂, R₅, a D or L amino acid or a peptide.

In preferred compounds of Formula I wherein nucleobase Bx has the Formula XVI, each Bx is independently selected from the group consisting of a radical of formula XVI, adeninyl, guaninyl, thyminyl, cytosinyl, uracilyl, 5-methylcytosinyl (5-me-C), 5-hydroxymethyl cytosinyl, xanthinyl, hypoxanthinyl, 2-aminoadeninyl, alkyl derivatives of adeninyl and guaninyl, 2-thiouracilyl, 2-thiothyminyl, 2-thiocytosinyl, 5-halouracilyl, 5-halocytosinyl, 5-propynyl uracilyl, 5-propynyl cytosinyl, 6-azo uracilyl, 6-azo cytosinyl, 6-azo thyminyl, 5-uracilyl (pseudouracil), 4-thiouracilyl, 8-substituted adeninyls and guaninyls, 5-substituted uracilyls and cytosinyls, 7-methylguaninyl, 7-methyladeninyl, 8-azaguaninyl, 8-azaadeninyl, 7-deazaguaninyl, 7-deazaadeninyl, 3-deazaguaninyl and 3-deazaadeninyl.

The PNAs of the present invention include compounds of Formula X, XI, XII, XIII, XIV, and XV wherein nucleobase Bx has one of the Formulas II or III. These nucleobases are attached to the PNA backbone by a suitable linker.

In preferred compounds, R₁ is —CH₂—Q₁. Q₁ is —N₃, —CN, —N(Z₁)Z₂, —N(Z₁)—(CH₂)ₙ—C(═NH)—N(H)—Z₃, —N(Z₁)—C(═J)—N(H)—Z₅, —L—(CH₂)ₙ—C(═O)Z₃, —L—(CH₂)ₙ—L—Z₃, —L—(CH₂)ₙ—N(H)Z₁ , —L—(CH₂)ₙ—N(Z₁)—(CH₂)ₙ—N(H)Z₁, —L—(CH₂)ₙ—C(═NH)N(Z₁)Z₃ or —L—(CH₂)ₙ—N(Z₁)—C(═J)—N(H)Z₃.

In other preferred embodiments, R₁ is —C≡C—Q₂. Q₂ is H, methyl, ethyl, —C(═O)—N(H)Z₁, —CH₂—N(H)—Z₂ or —CH₂—N(H)—C(═NH)—N(H)—Z₅.

In still other preferred embodiments, R₁ is —CH₂—(CH₂)ₙ—Q₃. Q₃ is hydrogen, —O—-CH₃, —O—CH₂CH₃, —N(H)—Z₁, —N(H)—Z₂, —N(H)—C(═O)—CF₃ or —N(H)—C(═NH)—N(H)Z₁. In another embodiment, Q₃ is —N(H)—C(═O)—(CH₂)ₙ—Q₅, —O—N(H)—C(═O)—(CH₂)ₙ—Q₅ or —C(═O)—N(H)—(CH₂)ₙ—Q₅ and Q₅is —N(H)Z₃, —C(═NH)—N(H)Z₃ or —N(H)—C(═J) N(H)Z₃. In another embodiment, Q₃ is —O—Q₆ and Q₆ is hydrogen, —N(H)Z₁or —N(H)Z₂.

In still other preferred embodiments, each R₁ is —CH═CH—C(═O)—Q₄. Q₄ is —OH, —N(H)Z₃, —C₁-C₆ alkyl, —O—C₁-C₆ alkyl, —O-benzyl or —N(H)—(CH₂)ₙ—Q₅. In another embodiment, Q₄ is —N(H)Z₃ and Z₃ is Hydrogen or C₁-C₆ alkyl.

Preferrably, Z₁, Z₂, Z₃, Z₄ and Z₅ are each independently hydrogen, methyl or an amino protecting group. Each n is independently from 1 to about 3.

In still other preferred embodiments, T₁ is hydrogen, an amino protecting group, a reporter group or a D or L amino acid or a peptide. More preferably, D or L amino acid is lysine or glutamic acid.

In still other preferred embodiments, T₂ is —OH, —N(Z₁)Z₂, R₅ or a D or L amino acid or a peptide.

In still other preferred compounds of Formula X, XI, XII, XIII, XIV, and XV, each Bx is independently selected from the group consisting of a radical of formula II, formula III, adeninyl, guaninyl, thyminyl, cytosinyl, uracilyl, 5-methylcytosinyl (5-me-C), 5-hydroxymethyl cytosinyl, xanthinyl, hypoxanthinyl, 2-aminoadeninyl, alkyl derivatives of adeninyl and guaninyl, 2-thiouracilyl, 2-thiothyminyl, 2-thiocytosinyl, 5-halouracilyl, 5-halocytosinyl, 5-propynyl uracilyl, 5-propynyl cytosinyl, 6-azo uracilyl, 6-azo cytosinyl, 6-azo thyminyl, 5-uracilyl (pseudouracil), 4-thiouracilyl, 8-substituted adeninyls and guaninyls, 5-substituted uracilyls and cytosinyls, 7-methylguaninyl, 7-methyladeninyl, 8-azaguaninyl, 8-azaadeninyl, 7-deazaguaninyl, 7-deazaadeninyl, 3-deazaguaninyl and 3-deazaadeninyl.

Preferrably compounds of Formula X, XI, XII, XIII, XIV, and XV wherein nucleobase Bx has one of the Formulas II or III are prepared having substantially pure R or S configuration at each of said chiral ring carbons. In other preferred embodiments these compounds are prepared with essentially equal amounts of R and S configuration at each of said chiral ring carbons.

The PNAs of the present invention include compounds of Formula X, XI, XII, XIII, XIV, and XV wherein nucleobase Bx has one of the Formulas V or VI. These nucleobases are attached to the PNA backbone by a suitable linker.

In preferred embodiments of these compounds, R₂ is hydrogen and R₃ is Z₁, —C(═J)—N(H)Z₁or —(CH₂)ₙ—C(═NH)—N(H)Z₃.

In other preferred embodiments, R₂ is —C(═O)—(CH₂)ₙ—L—Z₉ and Z₉ is hydrogen, —C₁-C₃ alkyl or —C(═O)—CH₃.

In still other preferred embodiments, R₃ is hydrogen and R₂ is —C≡C—R₄ or —(CH₂)ₘ—R_{4.} R₄ is H, C₁-C₃ alkyl, —CH₂OH, —CH₂—O—Q₆, —CH₂—N(H)Z₂ or —C(═O)—Z₄. In another embodiment, R₄ is —C(═O)—Z₄, —C(═O)—N(H)—(CH₂)ₙ—Q₅, —CH₂—N(H)—C(═O)—(CH₂)ₙQ₅ or —CH₂—O—N(H)—C(═O)—(CH₂)ₙ—Q₅ and Q₅ is —N(H)Z₁or —C(═NH)—N(H)Z₃. In still another embodiment, R₄ is —CH₂—O—Q₆ and Q₆ is —N(H)Z₂, —C(═O)—(CH₂)ₙ—CH₃ or phthalimido. In yet another embodiment, R₄ is —C(═O)—Z₄ and Z₄ is —OH, C₁-C₃ alkyl, benzyl or —N(H)Z₁.

In still other preferred embodiments, R₃ is —(CH₂)ₙ—N(H)—C(═J)—N(H)Z₃ or —(CH₂)ₙ—C(═NH)—N(H)Z₃ and Z₃ is hydrogen, an amino protecting group, —C₁-C₃ alkyl or —C(═O)—CH₃.

In still other preferred embodiments, T₁ is hydrogen, an amino protecting group, a reporter group, a D or L amino acid or a peptide. Preferably, D or L amino acid is lysine or glutamic acid.

In still other preferred embodiments, T₂ is —N(Z₁)Z₂ and Z₂ is hydrogen, C₁-C₃ alkyl, an amino protecting group. In yet another emboidment, T₂ is —OH, —(Z₁)Z₂, R₅, a D or L amino acid or a peptide.

In another preferred embodiment, each Bx is independently selected from the group consisting of a radical of formula V, formula VI, adeninyl, guaninyl, thyminyl, cytosinyl, uracilyl, 5-methylcytosinyl (5-me-C), 5-hydroxymethyl cytosinyl, xanthinyl, hypoxanthinyl, 2-aminoadeninyl, alkyl derivatives of adeninyl and guaninyl, 2-thiouracilyl, 2-thiothyminyl, 2-thiocytosinyl, 5-halouracilyl, 5-halocytosinyl, 5-propynyl uracilyl, 5-propynyl cytosinyl, 6-azo uracilyl, 6-azo cytosinyl, 6-azo thyminyl, 5-uracilyl (pseudouracil), 4-thiouracilyl, 8-substituted adeninyls and guaninyls, 5-substituted uracilyls and cytosinyls, 7-methylguaninyl, 7-methyladeninyl, 8-azaguaninyl, 8-azaadeninyl, 7-deazaguaninyl, 7-deazaadeninyl, 3-deazaguaninyl and 3-deazaadeninyl.

Preferrably compounds of Formula X, XI, XII, XIII, XIV, and XV wherein nucleobase Bx has one of the Formulas V or VI are prepared having substantially pure R or S configuration at each of said chiral ring carbons. In other preferred embodiments these compounds are prepared with essentially equal amounts of R and S configuration at each of said chiral ring carbons.

The PNAs of the present invention include compounds of Formula X, XI, XII, XIII, XIV, and XV wherein nucleobase Bx has Formulas VIII. These nucleobases are attached to the PNA backbone by a suitable linker.

In preferred embodiments of these compounds, A₁₃ is H; A₁₂ is —O—(CH₂)₂—N(H)G₄, —O—(CH₂)₂—ON(H)G₄ or —O—(CH₂)₂—C(═NH)N(H)G₄, —O—(CH₂)₃—C(═NH)N(H)G₄, —O—(CH₂)₂—C(═O)N(H)G₄, —O—(CH₂)₂—C(═S)N(H)G₄ or —O—(CH₂)₂—N(H)C(═NH)N(H)G₄; and G₄ is hydrogen, an amino protecting group or C₁-C₁₀ alkyl. In one embodiment A₁₀ is S. In another embodiment, A₁₁ is O.

In other preferred embodiments, T₁ is hydrogen, an amino protecting group, a reporter group, a D or L amino acid or a peptide.

In still other preferred embodiments, D or L amino acid is lysine or glutamic acid.

In still other preferred embodiments, T₂ is —OH, —(Z₁)Z₂, R₅, a D or L amino acid or a peptide.

Preferrably in compounds of Formula X, XI, XII, XIII, XIV, and XV wherein nucleobase Bx has Formulas VIII, each Bx is independently selected from the group consisting of a radical of formula VIII, adeninyl, guaninyl, thyminyl, cytosinyl, uracilyl, 5-methylcytosinyl (5-me-C), 5-hydroxymethyl cytosinyl, xanthinyl, hypoxanthinyl, 2-aminoadeninyl, alkyl derivatives of adeninyl and guaninyl, 2-thiouracilyl, 2-thiothyminyl, 2-thiocytosinyl, 5-halouracilyl, 5-halocytosinyl, 5-propynyl uracilyl, 5-propynyl cytosinyl, 6-azo uracilyl, 6-azo cytosinyl, 6-azo thyminyl, 5-uracilyl (pseudouracil), 4-thiouracilyl, 8-substituted adeninyls and guaninyls, 5-substituted uracilyls and cytosinyls, 7-methylguaninyl, 7-methyladeninyl, 8-azaguaninyl, 8-azaadeninyl, 7-deazaguaninyl, 7-deazaadeninyl, 3-deazaguaninyl and 3-deazaadeninyl.

Preferrably compounds of Formula X, XI, XII, XIII, XIV, and XV wherein nucleobase Bx has Formulas VIII are prepared having substantially pure R or S configuration at each of said chiral ring carbons. In other preferred embodiments these compounds are prepared with essentially equal amounts of R and S configuration at each of said chiral ring carbons.

PNAs are useful in a number of different areas because they often have stronger binding and greater specificity than oligonucleotides. Therefore they are used as probes in cloning, blotting procedures, and in applications such as fluorescence in situ hybridization (FISH).

Homopyrimidine PNAs are used for strand displacement in homopurine targets. The restriction sites that overlap with or are adjacent to the D-loop will not be cleaved by restriction enzymes. Also, the local triplex inhibits gene transcription. Thus in binding of PNAs to specific restriction sites within a DNA fragment, cleavage at those sites can be inhibited. Advantage can be taken of this in cloning and subcloning procedures. Labeled PNAs are also used to directly map DNA molecules. In effecting this, PNA molecules having a fluorescent label are hybridized to complementary sequences in duplex DNA using strand invasion.

The PNAs of the present invention can be used for gene modulation (e.g., gene targeted drugs), diagnostics, biotechnology and other research purposes. The PNAs can also be used to target RNA and single-stranded DNA (ssDNA) to produce both antisense-type gene regulating moieties and as hybridization probes, e.g., for the identification and purification of nucleic acids. Furthermore, the PNAs can be modified in such a way that they form triple helices with double stranded DNA (dsDNA). Compounds that bind sequence-specifically to dsDNA have applications as gene targeted drugs. These compounds are extremely useful drugs for treating various diseases, including cancer, acquired immune deficiency syndrome (AIDS) and other virus infections and genetic disorders. Furthermore, these compounds can be used in research, diagnostics and for detection and isolation of specific nucleic acids.

Gene-targeted drugs are designed with a nucleobase sequence (preferably containing 10-20 units) complementary to the regulatory region (the promoter) of the target gene. Therefore, upon administration, the gene-targeted drugs bind to the promoter and prevent RNA polymerase from accessing the promoter. Consequently, no MRNA, and thus no gene product (protein), is produced. If the target is within a vital gene for a virus, no viable virus particles will be produced. Alternatively, the target region could be downstream from the promoter, causing the RNA polymerase to terminate at this position, thus forming a truncated mRNA/protein which is nonfunctional.

Likely therapeutic and prophylactic targets include herpes simplex virus (HSV), human papillomavirus (HPV), human immunodeficiency virus (HIV), candida albicans, influenza virus, cytomegalovirus (CMV), intercellular adhesion molecules (ICAM), 5-lipoxygenase (5-LO), phospholipase A₂ (PLA₂), protein kinase C (PKC), and the ras oncogene. Potential treatment of such targeting include ocular, labial, genital, and systemic herpes simplex I and II infections; genital warts; cervical cancer; common warts; Kaposi's sarcoma; AIDS; skin and systemic fungal infections; flu; pneumonia; retinitis and pneumonitis in immunosuppressed patients; mononucleosis; ocular, skin and systemic inflammation; cardiovascular disease; cancer; asthma; psoriasis; cardiovascular collapse; cardiac infarction; gastrointestinal disease; kidney disease; rheumatoid arthritis; osteoarthritis; acute pancreatitis; septic shock; and Crohn's disease.

In general, for therapeutic or prophylactic treatment, a patient suspected of requiring such therapy is administered a PNA composition of the present invention, commonly in a pharmaceutically acceptable carrier, in amounts and for periods of time which will vary depending upon the nature of the particular disease, its severity and the patient's overall condition. The PNAs and liposomal compositions of the invention can be formulated in a pharmaceutical composition, which may include carriers, thickeners, diluents, buffers, preservatives, surface active agents and the like. Pharmaceutical compositions may also include one or more active ingredients such as antimicrobial agents, anti-inflammatory agents, anesthetics and the like, in addition to the peptide nucleic acids.

The pharmaceutical composition may be administered in a number of ways depending upon whether local or systemic treatment is desired, and upon the area to be treated. Administration may be topical (including ophthalmic, vaginal, rectal, intranasal, transdermal), oral or parenteral, for example, by intravenous drip, subcutaneous, intraperitoneal or intramuscular injection or intrathecal or intraventricular administration.

Formulations for topical administration may include transdermal patches, ointments, lotions, creams, gels, drops, suppositories, sprays, liquids and powders. Conventional pharmaceutical carriers, nucleic acid carriers, aqueous, powder or oily bases, thickeners and the like may be necessary or desirable in certain circumstances. Coated condoms, gloves and the like may also be useful. Topical administration also includes delivery of the PNAs and liposomal compositions of the invention into the epidermis of an animal by electroporation. Zewart et al., WO 96/39531, published Dec. 12, 1996.

Compositions for oral administration include powders or granules, suspensions or solutions in aqueous or non-aqueous media, capsules, sachets, or tablets. Thickeners, flavorings, diluents, emulsifiers, dispersing aids or binders may be desirable.

Intravitreal injection, for direct delivery of the PNAs and liposomal compositions of the invention to the vitreous humor of the eye of an animal is described in U.S. Pat. No. 5,595,978, issued Jan. 21, 1997, the contents of which are herein incorporated by reference.

Intraluminal administration, for direct delivery of PNAs and liposomal compositions of the invention to an isolated portion of a tubular organ or tissue (e.g., artery, vein, ureter or urethra) may be desired for the treatment of patients with diseases or conditions afflicting the lumen of such organs or tissues. To effect this mode of administration, a catheter or cannula is surgically introduced by appropriate means. After isolation of the portion of the tubular organ or tissue for which treatment is sought, the PNA or liposomal composition of the invention is infused through the catheter or cannula. The infusion catheter or cannula is then removed, and flow within the tubular organ or tissue is restored by removal of the ligatures which effected the isolation of a segment thereof. Morishita et al., Proc. Natl. Acad. Sci., U.S.A., 1993, 90, 8474.

Intraventricular administration, for direct delivery of PNAs or liposomal compositions of the invention to the brain of a patient, may be desired for the treatment of patients with diseases or conditions afflicting the brain. To effect this mode of administration, a silicon catheter is surgically introduced into a ventricle of the brain, and is connected to a subcutaneous infusion pump (Medtronic, Inc., Minneapolis, Minn.) that has been surgically implanted in the abdominal region. Zimm et al., Cancer Research, 1984, 44, 1698; and Shaw, Cancer, 1993, 72(11 Suppl.), 3416. The pump is used to inject the PNA or liposomal composition, and allows precise dosage adjustments and variation in dosage schedules with the aid of an external programming device. The reservoir capacity of the pump is 18-20 mL, and infusion rates may range from 0.1 mL/hour to 1 mL/hour. Depending on the frequency of administration, ranging from daily to monthly, and the dose to be administered, ranging from 0.01□μg to 100 g per kg of body weight, the pump reservoir may be refilled at 3-10 week intervals. Refilling of the pump is accomplished by percutaneous puncture of the self-sealing septum of the pump. Compositions for intraventricular administration may include sterile aqueous solutions which may also contain buffers, diluents and other suitable additives.

Intrathecal administration, for the direct delivery of PNAs compositions of the invention into the spinal column of a patient, may be desired for the treatment of patients with diseases of the central nervous system. To effect this route of administration, a catheter is surgically implanted into the L3-4 lumbar spinal interspace of the patient, and is connected to a subcutaneous infusion pump which has been surgically implanted in the upper abdominal region. Luer and Hatton, The Annals of Pharmacotherapy, 1993, 27, 912; Ettinger et al., Cancer, 1978, 41, 1270; and Yaida et al., Regul. Pept., 1995, 59, 193. The pump is used to inject the PNA, and allows precise dosage adjustments and variations in dose schedules with the aid of an external programming device. The reservoir capacity of the pump is 18-20 mL, and infusion rates may vary from 0.1 mL/hour to 1 mL/hour. Depending on the frequency of administration, ranging from daily to monthly, and dosage to be administered, ranging from 0.01 μg to 100 g per kg of body weight, the pump reservoir may be refilled at 3-10 week intervals. Refilling of the pump is accomplished by a single percutaneous puncture to the self-sealing septum of the pump. Compositions for intrathecal administration may include sterile aqueous solutions which may also contain buffers, diluents and other suitable additives.

To effect delivery to areas other than the brain or spinal column via this method, the silicon catheter may be configured to connect the subcutaneous infusion pump to, e.g., the hepatic artery, for delivery to the liver. Kemeny et al., Cancer, 1993, 71, 1964. Infusion pumps may also be used to effect systemic delivery. Ewel et al., Cancer Research, 1992, 52, 3005; and Rubenstein et al., J. Surg. Oncol., 1996, 62, 194.

Compositions for parenteral, intrathecal or intraventricular administration, or liposomal systems, may include sterile aqueous solutions which may also contain buffers, diluents and other suitable additives. Dosing is dependent on severity and responsiveness of the disease state to be treated, with the course of treatment lasting from several days to several months, or until a cure is effected or a diminution of the disease state is achieved. Optimal dosing schedules can be calculated from measurements of drug accumulation in the body of the patient. Persons of ordinary skill can easily determine optimum dosages, dosing methodologies and repetition rates. Optimum dosages may vary depending on the relative potency of individual PNAs, and can generally be estimated based on EC₅₀s found to be effective in in vitro and in vivo animal models. In general, dosage is from 0.01 μg to 100 g per kg of body weight, and may be given once or more daily, weekly, monthly or yearly, or even once every 2 to 20 years.

Peptide nucleic acids (PNAs) are prepared in accordance with any of the various procedures referred to in Peptide Nucleic Acids (PNA): Synthesis, Properties and Potential Applications, Bioorganic & Medicinal Chemistry, 1996, 4, 5-23. They may also be prepared in accordance with U.S. Pat. Nos. 5,539,082, 5,700,922, and 5,719,262, herein incorporated by reference.

Additional objects, advantages, and novel features of the present invention will become apparent to those skilled in the art upon examination of the following examples thereof. The following examples illustrate the invention and are not intended to limit the same. Those skilled in the art will recognize, or be able to ascertain through routine experimentation, numerous equivalents to the specific substances, compositions, and procedures described herein. Such equivalents are considered to be within the scope of the present invention.

### EXAMPLES

The following abbreviations are used in the experimental examples: eg1, —NH—CH₂—CH₂—O—CH₂—CH₂—O—CH₂—C(═O)—; Aha, 6-amino hexanoic acid; DMF, N,N-dimethylformamide; DCC, N,N-dicyclohexyl carbodiimide; DCU, N,N-dicyclohexyl urea; THF, tetrahydroflran; aeg, (2′-aminoethyl)glycine; Tyr, tyrosine; Lys, lysine;

DCC, N,N-dicyclohexyl-carbodiimide; aek, N-acetyl—N—(2′-aminoethyl)lysine; Pfp, pentafluorophenyl; BOC, tert-butoxycarbonyl; Z, benzyloxycarbonyl; NMR, nuclear magnetic resonance; s, singlet; d, doublet; dd, doublet of doublets; t; triplet; q, quartet; m, multiplet; b, broad; δ, chemical shift; ppm, parts per million (chemical shift).

NMR spectra were recorded on JEOL FX 90Q spectrometer or a Bruker 250 MHz with tetramethylsilane as an internal standard. Mass spectrometry was performed on a MassLab VG 12-250 quadropole instrument fitted with a VG FAB source and probe. Melting points were recorded on a Buchi melting point apparatus and are uncorrected. N,N-Dimethylformamide was dried over 4 Å molecular sieves, distilled and stored over 4 Å molecular sieves. Pyridine (HPLC quality) was dried and stored over 4 Å molecular sieves. Other solvents used were either the highest quality obtainable or were distilled prior to use. Dioxane was passed through basic alumina prior to use. BOC-anhydride, 4-nitrophenol, methyl bromoacetate, benzyloxycarbonyl chloride, pentafluorophenol were all obtained from Aldrich Chemical Company. Thymine, cytosine, adenine were all obtained from Sigma.

Thin layer chromatography (tlc) was performed using the following solvent systems: (1) chloroform:triethyl amine:methanol, 7:1:2; (2) methylene chloride:methanol, 9:1; (3) chloroform:methanol:acetic acid 85:10:5. Spots were visualized by UV (254 nm) and/or spraying with a ninhydrin solution (3 g ninhydrin in 1000 mL of 1-butanol and 30 mL of acetic acid), after heating at 120 degrees Centigrade for 5 minutes and, after spraying, heating again.

The carboxyl terminal (C terminus) end of PNA oligomers can be substituted with a variety of functional groups. One way this is performed is through the use of different resins. The amino terminal (N terminus) end of PNA oligomers can also be capped with a carboxylic acid-based capping reagent for the final PNA monomer in the final coupling step, or substituted with a variety of conjugate groups. Representative examples of the types of C and N terminal groups are shown below.

| | aeg-PNA/aeg- |
|---|---|
| Resin Employed | PNA Derivative Prepared |
| (Capping Reagent = Acetyl) | |
| Merrifield | CH₃CONH-(PNA)-COOH |
| | H₂N-(PNA)-COOH |
| Lys Substituted Merifield | H₂N-(PNA)-Lys-COOH |
| Merrifield | H₂N-(PNA)-CONH₂ |
| Lys Substituted MBHA | H₂N-(PNA)-Lys-CONH₂ |
| Lys Substituted Merrifield | CH₃CONH-(PNA)-Lys-COOH |
| | H₂N-(PNA)-COOH |
| Lys Substituted Merrifield | H₂N-(PNA)-Lys-COOH |
| Merrifield | H₂N-(PNA)-CONH₂ |
| MBHA | H₂N-(PNA)-CONH₂ |
| Lys Substituted MBHA | H₂N-(PNA)-Lys-CONH₂ |
| MBHA | CH₃CONH-(PNA)-CONH₂ |
| | H₂N-(PNA)-CONH₂ |
| Lys Substituted MBHA | CH₃CONH-(PNA)-Lys-CONH₂ |
| (Capping Reagent = N-Boc glycine) | |
| Merrifield | BocGly-(PNA)-COOH |
| Lys Substituted Merrifield | BocGly-(PNA)-Lys-COOH |
| MBHA | BocGly-(PNA)-CONH₂ |
| Lys Substituted MBHA | BocGly-(PNA)-Lys-CONH₂ |
| (Capping Reagent = 1. Glycine; | |
| 2. Cholic Acid (Chol)) | |
| Merrifield | Chol-Gly-(PNA)-COOH |
| Lys Substituted Merrifield | Chol-Gly-(PNA)-Lys-COOH |
| MBHA | Chol-Gly-(PNA)-CONH₂ |
| Lys Substituted MBHA | Chol-Gly-(PNA)-Lys-CONH₂ |

Other resins known to those skilled in the art can also be employed.

### Example 1

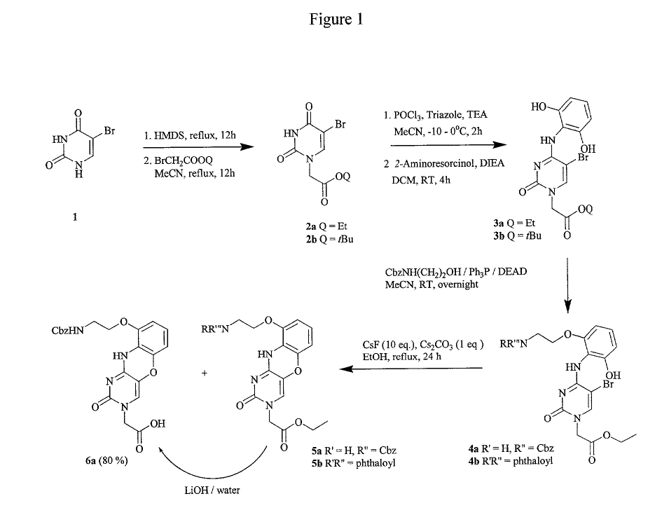

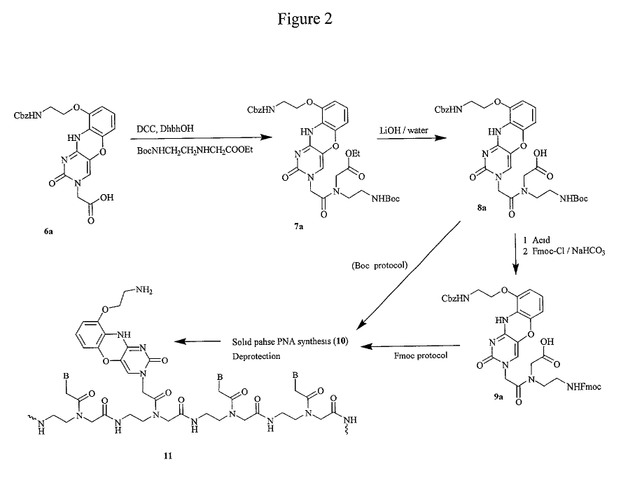

Synthesis of Compound 2a FIG. 1 shows a representative synthesis of tricyclic compound 6a. FIG. 2 shows preparation of PNA oligomer 11. Referring to FIGS. 1 & 2, a suspension of 5-bromouracil (1, 25.0 g, 130.89 mmol) in neat HMDS (100 mL) was refluxed for 24 h, resulting in the formation of clear solution of the trimethylsilylated derivative of 1. After cooling to room temperature, excess HMDS was removed from the reaction mixture under vacuum to obtain the silyl derivative as pale yellow oil. The residue obtained was dissolved in anhydrous acetonitrile (80 mL) and ethyl bromoacetate (29 mL, 261.51 mmol, 2 molar eq.) was added dropwise into the solution under constant stirring at room temperature in argon atmosphere. The addition was completed in 30 min and the resulting solution was refluxed for 6 h. The reaction was complete after 6 h of reflux as evident from TLC. After cooling to room temperature, acetonitrile, excess ethyl bromoacetate and TMS-Br were removed under vacuum. The residue was suspended in saturated bicarbonate solution (100 mL), stirred for five min and then filtered. After a bicarbonate wash, residue in warm water (100 mL) was stirred for 20 min and then washed extensively with warm water under suction. The white sticky residue obtained was triturated with diethyl ether, filtered and dried to a white solid (34.2 g, 94% yield). ¹H NMR (200 MHz, DMSO-d₆): δ 11.94 (s, 1H, exchangeable with D₂O), 8.22 (s, 1H), 4.50 (s, 2H), 4.09-4.20 (q, 2H), 1.47-1.51 (t, 3H). ¹³C NMR (50 MHz, DMSO-d₆): δ 167.9, 169.6, 150.3, 145.5, 94.8, 61.4, 48.7, 14.0. Mass Calc for C₈H₉BrN₂O₄: 277.07; Found, 276.8 (100) & 275.9 (negative ion) and 276.9 (positive ion)

### Example 2

Synthesis of compound 2b. Compound 2b is prepared by the reaction of t-butyl bromoacetate (2 mol) with silylated 5-bromouracil (5-bromouracil, 1 mol) as described in Example 1.

### Example 3

Synthesis of Compound 3a. Compound 2a (15.0 g, 54.15 mmol) and 1,2,4-triazole (40.0 g, 579.63 mmol, ca 10 molar eq.) were suspended in anhydrous acetonitrile (400 mL) under argon and stirred at B10° C. POCl₃ (10.1 mL, 108.36 mmol, 2 molar eq.) was added dropwise into the stirring solution by maintaining the temperature of the bath at B10° C. and the addition was completed in 20 min. After 20 min of the addition of POCl₃, precooled anhydrous TEA (85 mL, 609.84 mmol, cooled over a freezing mixture bath under argon) was added dropwise into the reaction mixture for a period of 30 min. The stirring was continued for 1.5 h at the same bath temperature. After bringing to room temperature, solvent was removed from the reaction mixture under vacuum and the residue suspended in dichloromethane (500 mL) was washed with water (200 mL) followed by saturated bicarbonate (200 mL) and finally with water. Organic layer was concentrated to a pale yellow solid, dried over anhydrous P₂O₅ under vacuum over night. The pale yellow solid was redissolved in anhydrous dichloromethane (200 mL) and anhydrous 2-aminoresorcinol hydrochloride (12.0 g, 74.30 mmol, ˜1.4 molar eq., dried over P₂O₅ overnight under vacuum) were added followed by dropwise addition of DIEA (26 mL, 149.26 mmol, 2 molar eq. w. r. t. the aminoresorcinol). The addition of DIEA was completed in 20 min and the resulting brownish reaction mixture was stirred for 4 h at ambient temperature. Dichloromethane and excess DIEA were removed under vacuum. Residue was suspended in dichloromethane (150 mL), saturated NaHSO₄ solution (150 mL) was added into the suspension and stirred vigorously. A yellow precipitate separated out from the suspension. The precipitate was filtered, washed extensively with water and dichloromethane (100 mL). The yellow solid thus obtained was then subjected to a fast methanol (20 mL) wash under suction followed by final wash with diethyl ether (100 mL) and dried under vacuum over P₂O₅ to obtain compound 3a as a yellow solid (16.95 g, 76.7% yield). ¹H NMR (200 MHz, DMSO-d₆): δ 9.78 (bs, 2H, exchangeable with D₂O), 8.17 (s, 1H), 8.11 (bs, 1H, exchangeable with D₂O), 6.86-6.95 (t, 1H), 6.35-6.38 (d, 2H), 4.48 (s, 2H), 4.07-4.18 (q, 2H), 1.15-1.22 (t, 3H). ¹³C NMR (50 MHz, DMSO-d₆): δ 168.2, 159.0, 153.6, 153.1, 146.8, 127.4, 113.3, 107.1, 86.7, 61.0, 49.8, 14.0. Mass Calc. for. C₁₄H₁₄BrN₃O₅: 384.18; Found, 381.9 (100) & 382.9 (negative ion).

### Example 4

Synthesis of compound 3b. Compound 3b is synthesized from compound 2b and 2-aminoresorcinol (1.4 molar eq.) under identical conditions described in Example 3 for the synthesis of compound 3a

### Example 5

Synthesis of compound 4a. Neat diethyl azodicarboxylate (DEAD, 0.6 mL, 3.81 mmol, 1.1 molar eq.) was added dropwise into a suspension of compound 3a (1.35 g, 3.52 mmol), Ph₃P (1.2 g, 4.58 mmol, 1.3 molar eq.) and benzyl N-(2-hydroxyethyl)carbamate (0.76 g, 3.89 mmol, 1.1 molar eq.) in anhydrous acetonitrile (15 mL) under constant stirring in argon atmosphere, at ambient temperature. At the end of the addition, the reaction mixture became homogeneous and was stirred for overnight. Compound 4a precipitated out of the reaction mixture during the course of the reaction. Removed acetonitrile and solid residue obtained was triturated with diethyl ether, filtered and dried under vacuum over P₂O₅ to obtain compound 4a as a yellowish white solid (1.6 g, 81% yield). ¹H NMR (200 MHz, DMSO-d₆): δ 9.84 (bs, 1H exchangeable with D₂O), 8.17 (bs, 2H, became a sharp singlet after D₂O exchange and accounted for 1H), 7.33 (bs, 5H), 7.01-7.10 (t, 1H), 6.50-6.56 (bm, 2H), 5.01 (s, 2H), 4.46 (s, 2H), 3.90-4.17 (m, 4H), 3.28 (bs, 2H, overlapped with water peak present in the solvent and became clearly visible after D₂O exchange). 1.15-1.22 (t, 3H). Mass Calc. for C₂₄H₂₅BrN₄O₇: 561.38; Found: 560.9 (100) & 559.9 (negative ion) and 562.9 (100) & 562.0 (positive ion).

### Example 6

Synthesis of compound 4b. The desired compound 4b was synthesized from compound 3a (3.22 g, 8.39 mmol) and N-(2-hydroxyethyl)phthalimide (1.68 g, 8.79 mmol) under identical conditions as in Example 5 using Mitsunobu reagent (Ph₃P: 2.9 g, 11.66 mmol; DEAD: 1.4 mL, 8.89 mmol). Compound 4b was isolated as a yellowish white solid, 3.42 g (73.2% yield). ¹H NMR (200 MHz, DMSO-d₆): δ 9.54 (s, 1H exchangeable with D₂O), 7.99 (s, 1H), 7.91 (s, 1H, exchangeable with D₂O), 7.77-7.80 (m, 4H), 7.00-7.09 (t, 1H), 6.47-6.55 (t, 2H), 4.29 (s, 2H), 4.04-4.18 (m, 4H), 3.85-3.90 (t, 2H), 1.14-1.21 (t, 3H). ¹³C NMR (50 MHz, DMSO-d₆): δ 168.2, 167.5, 159.5, 154.8, 154.1, 153.7, 146.2, 134.2, 131.5, 127.8, 122.9, 114.1, 109.4, 103.3, 86.7, 64.9, 61.0, 49.7, 37.0, 14.0. Mass Calc for C₂₄H₂₁BrN₄O₇: 557.35; Found: 556.9 (100) & 555.9 (negative ion) and 558.9 (100) & 557.9 (positive ion).

### Example 7

Synthesis of compound 5a: A suspension of compound 4a (1.13 g, 2.01 mmol), cesium fluoride (CsF: 3.06 g, 20.14 mmol, 10 molar eq.) and cesium carbonate (Cs₂CO₃: 0.35 g, 1.07 mmol) in absolute ethanol (10 mL) was refluxed under argon atmosphere for 24 h. Cyclization of compound 4a to compound 5a was complete after 24 h of reflux and formation of compound 5a was visualized on TLC by its characteristic fluorescence. After being cooled to room temperature, ethanol was removed from the reaction mixture. Dilute NaHSO₄ solution (10 mL) was added into a suspension of the residue in ethyl acetate (20 mL) and stirred vigorously. A yellow solid was separated out and was filtered off. Separated ethyl acetate layer from the aqueous layer, washed with water and concentrated to a solid. The residue from the organic layer was purified by silica gel column chromatography: eluent, dichloromethane/ethyl acetate (3:2) to obtain compound 5a as a gray white solid (0.4 g, 41.3%). The solid separated from the suspension was washed extensively with water followed by ethyl acetate (50 mL) and dried under vacuum over P₂O₅ to weigh 0.38 g. This solid was characterized by NMR and mass as the free acid 6a (41.7% yield). The overall yield of cyclization is 83% (combined yield of acid and ester).

Compound 5a: ¹H NMR (200 MHz, DMSO-d₆): δ□9.80 (bs, 1H, exchangeable with D₂O), 7.74 (bt, 1H, exchangeable with D₂O), 7.49 (s, 1H), 7.33 (s, 5H), 6.76-6.84 (t, 1H), 6.58-6.62 (d, 1H), 6.43-6.47 (d, 1H), 5.04 (s, 2H), 4.39 (s, 2H), 4.08-4.19 (q, 2H), 3.91-3.94 (bt, 2H), 3.40-3.43 (bm, 2H), 1.16-1.23 (t, 3H). ¹³C NMR (50 MHz, DMSO-d₆): δ□168.3, 156.1, 154.4, 153.9, 146.0, 142.2, 137.0, 128.3, 127.8, 126.0, 123.2, 107.9, 107.1, 68.0, 65.5, 61.0, 49.8, 14.0 (Note: one CH₂ peak was overlapped with DMSO peak and was confirmed by gHMQC and gHMBC experiments). Mass Calc for C₂₄H₂₄N₄O₇: 480.16; Found: 479.0 (100, negative ion) and 481.0 (100, positive ion)

Compound 6a: ¹H NMR (200 MHz, DMSO-d₆+D₂O): δ 7.71 (bs, 1H), 7.31 (bs, 5H), 6.78 (bs, 1H), 6.58 (bs, 1H), 6.46 (bs 1H), 5.01 (bs, 2H), 4.28 (bs, 2H), 3.91 (bs, 2H), 3.40 (bs, 2H). Mass Calc for C₂₂H₂₀N₄O₇: 452.13; Found: 451.0 (100) & 452.0 (negative ion) and 453.0 (100) & 454 (positive ion)

### Example 8

Synthesis of compound 5b: A suspension of compound 4b (2.4 g, 4.31 mmol) and cesium fluoride (CsF: 3.3 g, 21.72 mmol, 5 molar eq.) in absolute ethanol (50 mL) was refluxed under argon atmosphere over 60 h. Unlike compound 5a, compound 5b did not undergo complete cyclization even after refluxing over a period of 60 h. (Also it should be noted that (1) the amount of CsF was 5 molar equivalent and no cesium carbonate was added into the reaction, and (2) the reaction was performed under relatively high dilute condition). After 60 h, the reaction being cooled down to room temperature, ethanol was removed under vacuum. Residue was suspended in ethyl acetate and washed with bicarbonate solution (30 mL) followed by standard work up. The desired compound 5b was purified by silica gel column chromatography: eluent 1., dichloromethane/ethyl acetate (4:1): 0.25 g (unreacted 4b, 10.4%); eluent 2, dichloromethane/ethyl acetate (3:2): 1.15 g (compound 5b, white solid, 56%). ¹H NMR (200 MHz, DMSO-d₆): δ 9.39 (s, 1H, exchangeable with D₂), 7.88-7.91 (m, 5H), 7.45 (s, 1H), 6.76-6.83 (t, 1H), 6.63-6.68 (dd, 1H), 6.42-6.47 (d, 1H), 4.38 (s, 2H), 4.04-4.26 (m, 4H), 3.91-3.97 (t, 2H), 1.17-1.24 (t, 3H). Mass Calc for C₂₄H₂₀N₄O₇: 476.13; Found: 475.1 (100) & 476.1 (negative ion) and 477.1 (100) 7 478.1 (positive ion).

### Example 9

Synthesis of compound 6a: Saponification of compound 5a using LiOH followed by acidification of the reaction yielded the free carboxylic acid as a yellowish white solid.

### Example 10

Synthesis of compound 7a: A solution of the acid 6a (50 mg, 0.111 mmol), DhbhOH (30 mg, 0.184 mmol) and DCC (25 mg, 0.121 mmol) in dry DMF (2 mL) was stirred at ambient temperature for 2 h after which ethyl-N—(2-(t-butyloxycarbonylamino)ethyl)glycinate (40 mg, 0.163 mmol) was added. The reaction was stirred for 8 h. DCU was removed by filtration and DMF was removed under vacuum. The residue in ethyl acetate (10 mL) was washed with bicarbonate, water, bisulfate and finally with water, dried over anhydrous Na₂SO₄. After removing the solvent, residue was loaded on a preparative TLC (60 F₂₅₄ 2 mm) and the product was separated by eluting with 3% MeOH in DCM followed by a second elution with 5% MeOH in DCM to obtain compound 7a as a white solid (30 mg, 39.8% yield). ¹H NMR (200 MHz, DMSO-d₆): δ 9.78(bs, 1H, exchangeable with D₂O), 7.75 (bt, 1H, exchangeable with D₂O), 7.31-7.36 (bm, 6H), 6.92 (bt, 1H, exchangeable with D₂O), 6.75-6.83 (t, 1H), 6.58-6.62 (d, 1H), 6.41-6.45 (d, 1H), 5.04 (s, 2H), 4.60 & 4.42 (s, major and minor rotamer, 2H), 4.30 (s, 0.5H), 3.90-4.19 (m, 5.5H), 2.98-3.40 (m, 6H), 1.35 (s, 95), 1.12-1.24 (m, 3H). Mass Calc for C₃₃H₄₀N₆O₁₀: 680.28; Found: 679.1 (100) & 680.1 (negative ion) and 681.1 & 682.1 (positive ion).

### Example 11

Synthesis of compound 8a: A solution of compound 7a (25 mg, 0.03676 mmol) in 2 M LiOH (0.2 mL) and THF (0.2 mL) is stirred for 20 min at 10° C., after which THF is removed in vacuo and the aqueous layer is made acidic (H=ca 4). The precipitated acid is filtered washes extensively with water and dries under vacuum over P₂O₅ to obtain the desired acid 8a.

### Example 12

Synthesis of compound 9a: Compound 8a (1 mmol) is suspended in a 1:1 mixture of dichloromethane and trifluoroacetic acid (TFA, 10 mL) and stirs at ambient temperature for 20 min. Solvent is removed from the reaction mixture and the amine-TFA salt in water (10 ml) is stirred with Fmoc-C1 (1.3 mmol) in the presence of NaHCO₃ (2.5 mmol) for 2 h at ambient temperature. The Fmoc protected PNA monomer is precipitated by adjusting the pH to 4 with dilute HCl. The precipitated solid is washed extensively with water followed by drying under vacuum over P₂O₅ yields compound 9a.

### Example 13

Incorporation of monomer 8a into PNA 10: The support bound PNA is synthesized by following solid phase Boc protocol for PNA synthesis using Boc protected standard PNA monomers (purchased from PerSeptive Biosystems) and HATU in the presence of DIEA as coupling agent. Support to monomer ratio is 1:4 and the “G-clamp” monomer is incorporated at designed site via pre-activation of the carboxyl group of 8a using HATU and subsequent injection into the reaction vessel followed by standard protocol for coupling, washing and further extension of the chain.

### Example 14

Final deprotection of the PNA (11) and its purification: The support bound fully protected PNA is thoroughly washed with anhydrous dichloromethane and then subjects to TMS-I treatment for 5 min in DCM (Ihara et. al., J. Chem. Soc., PT 1, 1988, 1277). Washes off TMS-I and benzyl iodide after which follows the final deprotection of the PNA from the support and removal of all other base protection. The final compound (11) is purified by RP-HPLC and characterize by TOF-MALDI-MS.

### Example 15

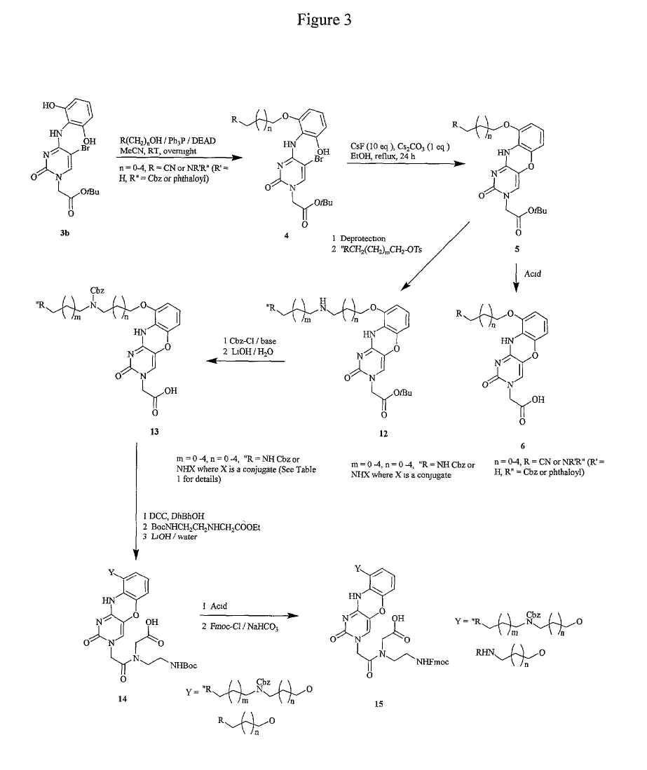

Synthesis of compound 4 (n=1, R=NHCbz): FIG. 3 shows a representative synthesis of tricyclic compound 15. Referring to FIG. 3, compound 4 (as specified) is prepared from compound 3b (1 mmol) and benzyl N-(3-hydroxypropyl)carbamate (from Aldrich, 1.1 mmol.) under Mitsunobu alkylation condition as described in Example 5 for the preparation of compound 4a.

### Example 16

Synthesis of compound 5 (n=1, R=NHCbz): Compound 5 (as specified) is prepared from compound 4 (1 mmol) by refluxing it with CsF (10 molar eq.) in absolute ethanol as described in Example 7 for the preparation of compound 5a.

### Example 17

Synthesis of compound 12 (m=0, n=1, AR=NHCbz): Compound 5 (1 mmol) in ethyl acetate is hydrogenated over 10% Pd-C at a pressure of 30 psi for 2 h. Filter off Pd-C from the product and the free amine thus obtained is dissolved in anhydrous DCM (5 ml) and stirred with N-(benzyloxycarbonyl)ethanolamine-O-tosylate (prepared from benzyl N-(2-hydroxyethyl)carbamate and tosyl chloride, one molar equivalent) in the presence of TEA at ambient temperature for over night. Reaction mixture is diluted to 20 mL and washed with saturated bicarbonate solution (5 mL) followed by standard work up. Compound 12 is purified by silica gel column chromatography.

### Example 18

Synthesis of compound 13 (m=0, n=1, AR=NHCbz): A solution of benzyl chloroformate (1. 1 mmol in 1 mL of DCM) is added into a cold solution of compound 12 (1 mmol) and DIEA (1.1 mmol) in DCM (5 mL) over an ice bath under constant stirring. After 30 min, reaction mixture is diluted to 20 mL by adding more DCM and subjects to bicarbonate (5 mL) and water wash. After removing DCM the residue is triturated with diethyl ether and filters. Residue obtained dissolves in a 1:1 mixture of DCM and TFA (5 mL) and stirs for 20 min. Removes DCM and TFA from the reaction and the solid residue is extensively washed with water, dries over P₂O₅ under vacuum to obtain compound 13 as a white solid.

### Example 19

Synthesis of compound 14 (m=0, n=1, AR=NHCbz): The title compound 14 is prepared by DCC and DhbhOH mediated coupling of ethyl-N-(2-(t-butyloxycarbonylamino)ethyl)glycinate (1 mmol) to the carboxylic group of compound 13 (1 mmol) as described in Example 11. Hydrolysis of the ethyl ester formed under alkaline condition gives compound 14.

### Example 20

Synthesis of compound 15 (m=0, n=1, AR=NHCbz): Compound 15 (as specified) is synthesized from compound 14 (1 mmol) as described in Example 12.

### Example 21

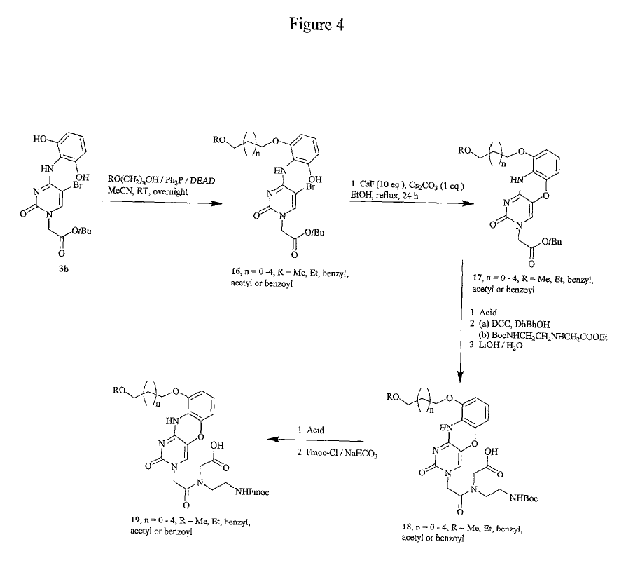

Synthesis of compound 16 (n=0, R=Me): FIG. 4 shows a representative synthesis of tricyclic compound 19. Referring to FIG. 4, compound 16 (as specified) is prepared from compound 3b (1 mmol) and 2-methoxyethanol (1 mmol) under Mitsunobu alkylation condition as in Example 5.

### Example 22

Synthesis of compound 17 (n=0, R=Me): Compound 17 (as specified) is prepared from compound 16 (1 mmol) and CsF (10 mmol) as described in Example 7 for the preparation of compound 5a.

### Example 23

Synthesis of compound 18 (n=0, R=Me): Compound 14 is prepared by DCC and DhbhOH mediated coupling of ethyl-N-(2-(t-butyloxycarbonylamino)ethyl)glycinate (1 mmol) to the carboxylic group of compound 17 (1.00 mmol) as described in Example 11. Alkaline hydrolysis of the ethyl ester thus obtained gives compound 18.

### Example 24

Synthesis of compound 19 (n=0, R=Me): Compound 19 is obtained from compound 18 by acid mediated deblocking of the t-Boc protection followed by reaction with Fmoc-Cl in the presence of NaHCO₃ as described in Example 12.

### Example 25

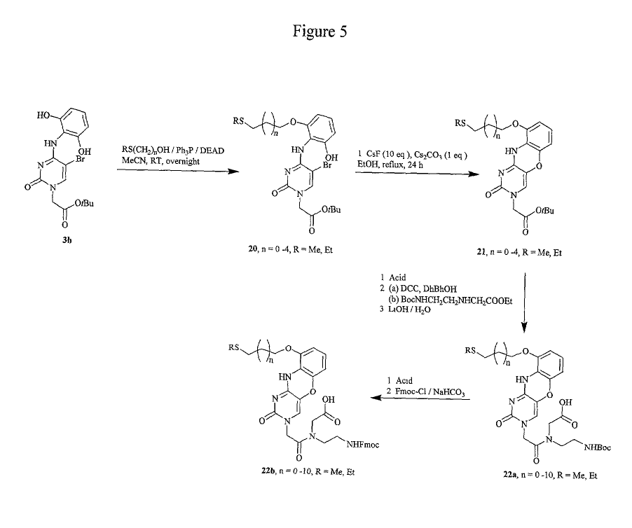

Synthesis of compound 22a (n=0, R=Me): FIG. 5 shows a representative synthesis of tricyclic compound 22b. Referring to FIG. 5, t-Boc protected PNA monomer 22a is synthesized from compound 3b and 2-(methylthio)ethanol as described in Examples 21, 22 and 23.

### Example 26

Synthesis of compound 22b (n=0, R=Me): Fmoc protected PNA monomer 22b is obtained from compound 22a as described in Example 24.

### Example 27

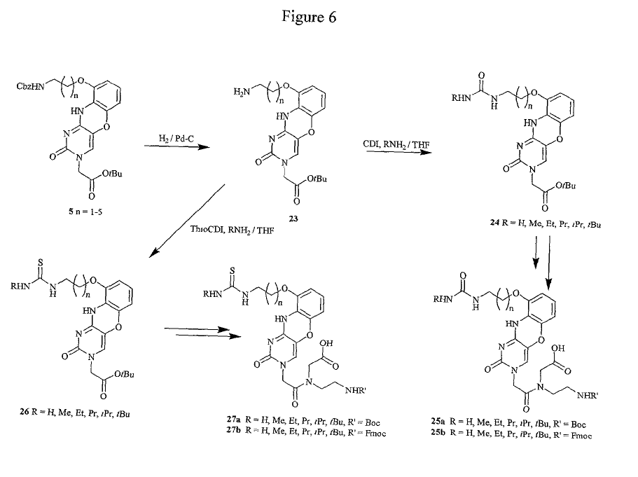

Synthesis of compound 23 (n=1): FIG. 6 shows a representative synthesis of tricyclic compounds 25a, 25b, 27a, and 27b. Referring to FIG. 6, compound 5 (1 mmol) is subjected to catalytic hydrogenation over Pd-C as described in Example 17 for the synthesis of compound 12 to obtain the free amine 23.

### Example 28

Synthesis of compound 24 (n=1, R=Me). Compound 23 (1 mmol) is stirred with 1,1′-carbonyldiimidazole (CDI from Aldrich, 1.1 mmol) in anhydrous THF (5 mL) under argon atmosphere at ambient temperature for 2 h. After 2 h, anhydrous methylamine is bubbled through the reaction mixture for 30 min at a temperature below 5° C. and stirs for 30 min. Standard works up and purification yields the desired urea derivative 24.

### Example 29

Synthesis of compound 25a (n=1, R=Me). t-Boc protected PNA monomer 25a is synthesized from compound 24 and ethyl-N-(2-(t-butyloxycarbonylamino)ethyl)glycinate as described in Example 10 for the synthesis of compound 7a.

### Example 30

Synthesis of compound 25b (n=1, R=Me). The Fmoc protected PNA monomer 25b is obtained from 25a as described in Example 12 for the synthesis of compound 9a from compound 8a.

### Example 31

Synthesis of compound 26 (n=1, R=Me): Compound 26 is prepared from compound 23 (1 mmol) and 1,1=-thiocarbonyldiimidazole (from Aldrich, 1.1 mmol) as described in Example 28 for the synthesis of compound 24.

### Example 32

Synthesis of compound 27a (n=1, R=Me). t-Boc protected PNA monomer 27a is synthesized from compound 26 and ethyl-N-(2-(t-butyloxycarbonylamino)ethyl)glycinate as described in Example 29 for the synthesis of compound 29a.

### Example 33

Synthesis of compound 27b (n=1, R=Me). The Fmoc protected PNA monomer 27b is obtained from 27a as described in Example 12 for the synthesis of compound 9a from compound 8a.

### Example 34

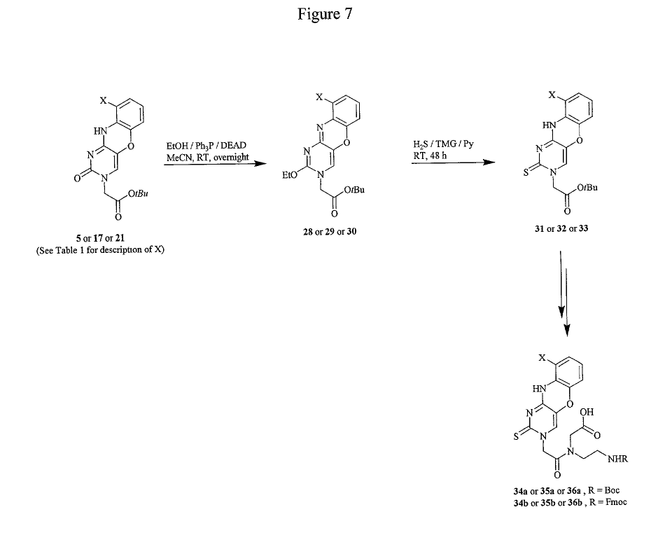

Synthesis of compound 28 (X=O—(CH₂)₂NHCbz): FIG. 7 shows a representative synthesis of tricyclic compounds 34a, 34b, 35a, 35b, 36a, and 36b. Referring to FIG. 7, neat DEAD (1.1 mmol) is added dropwise into a stirring solution of compound 5 (1 mmol), Ph₃P (1.2 mmol) and ethanol (1.2 mmol) in anhydrous MeCN (5 mL) at ambient temperature under argon. A molar equivalent of DIEA is added after 10 min of the addition of DEAD and stirs overnight to get the desired compound 28.

### Example 35

Synthesis of compound 31 (X=O—(CH₂)₂NHCbz): Compound 28 (1 mmol, dries over P₂O₅ overnight under vacuum) is placed in a sealed flask under argon. A precooled 10 molar equivalent of 1,1,3,3-tetramethylguanidine (TMG) in anhydrous pyridine is saturates with hydrogen sulfide for 30 min and transfers into the flask containing compound 28 under cold condition and under argon. The temperature of the reaction is slowly brought to room temperature and left at that temperature for 48 h to obtain compound 31.

### Example 36

Synthesis of compound 34a (X=O—(CH₂)₂NHCbz): Acid hydrolysis of compound 31 and subsequent coupling of the carboxylic function to ethyl-N-(2-(t-butyloxycarbonylamino)ethyl)glycinate using DCC and DhbhOH (Example 10) followed by alkaline hydrolysis (Example 11) of the ester formed, yields compound 34a.

### Example 37

Synthesis of compound 34b (X=O—(CH₂)₂NHCbz): The Fmoc protected PNA monomer 34b is obtained from 34a as described in Example 12 for the synthesis of compound 9a from compound 8a.

### Example 38

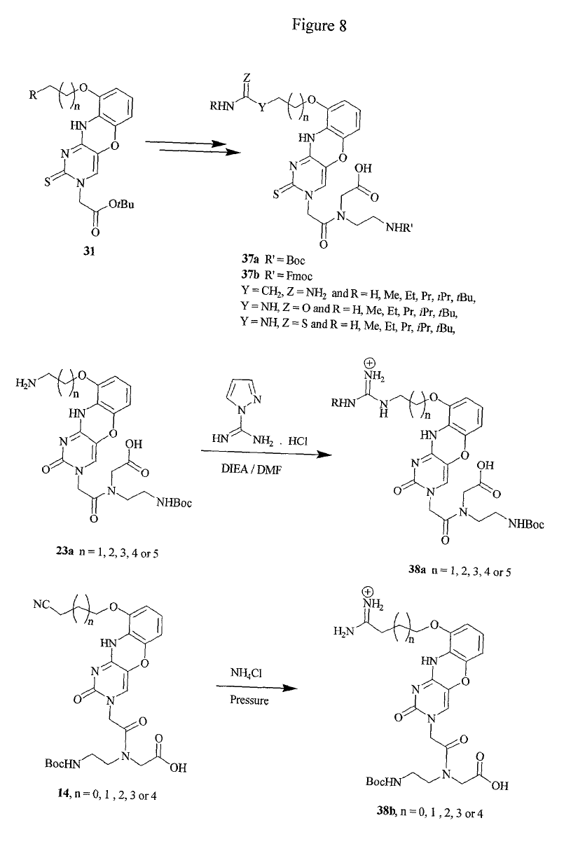

Synthesis of compound 37a (n=1, Y=NH, Z=O and R=H). FIG. 8 shows a representative synthesis of tricyclic compounds 37a, 37b, 38a, and 38b. Referring to FIG. 8, compound 37a is prepared from compound 31 (R=NH₂, n=1) and ammonia as described in Examples 28 and 29.

### Example 39

Synthesis of compound 38a (n=1). A solution of compound 23a (1 mmol), 1H-pyrazole-1-carboxamide hydrochloride (1 mmol) and DIEA (1 mmol) in anhydrous DMF (5 mL) is stirred at ambient temperature under argon for 6-8 h (Bematowics et. al., J Org. Chem. 1992, 57, 2497). After the reaction is done, the desired product is precipitated out by the addition of diethyl ether into the reaction to obtain compound 38a.

### Example 40

Synthesis of compound 38b (n=1). Compound 14 (1 mmol) and ammonium chloride as reported by Granik (Russ. Chem. Rev. 1983, 52, 377).

Referring to Examples 1-40, Table 6 shows representative tricyclic structures:

TABLE 6

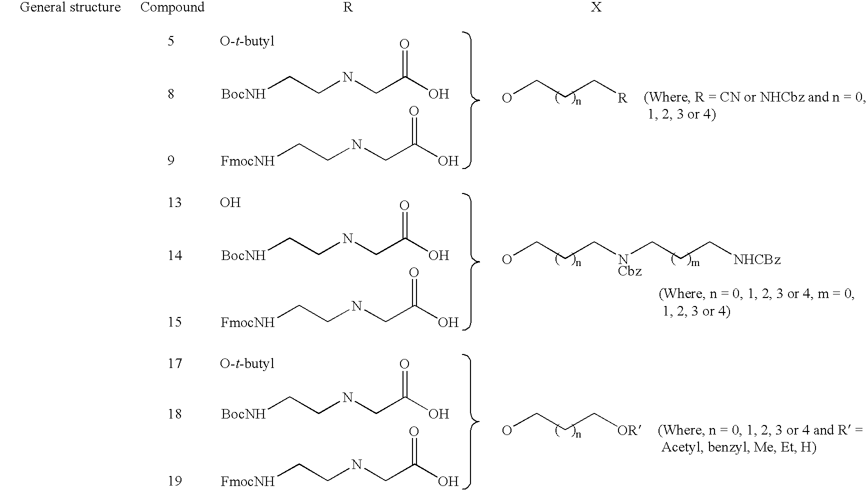

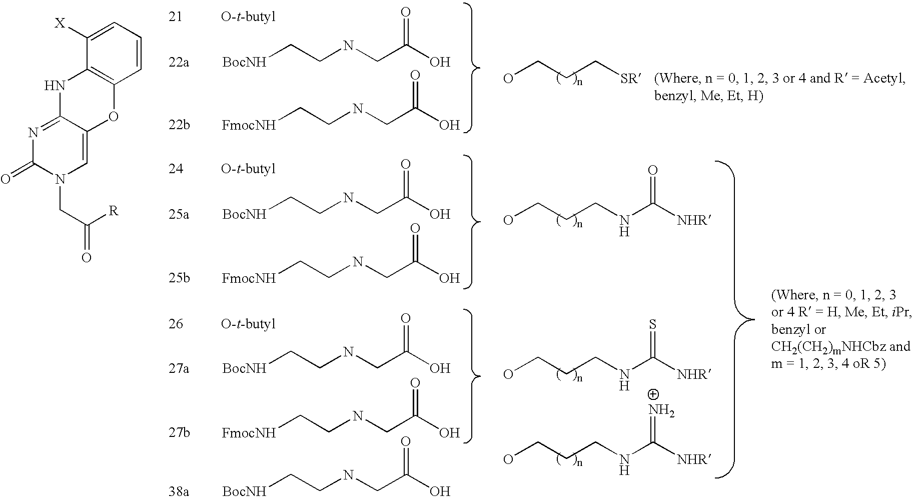

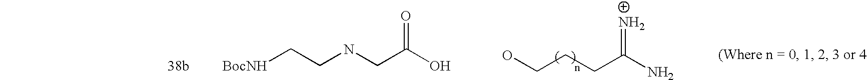

Referring to Examples 1-40, Table 7 shows further representative tricyclic structures:

TABLE 7

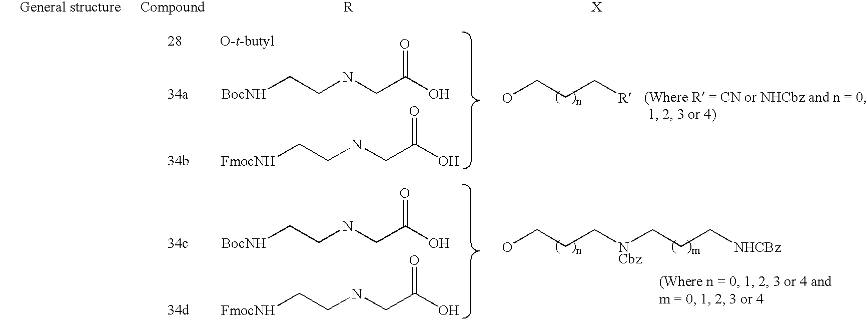

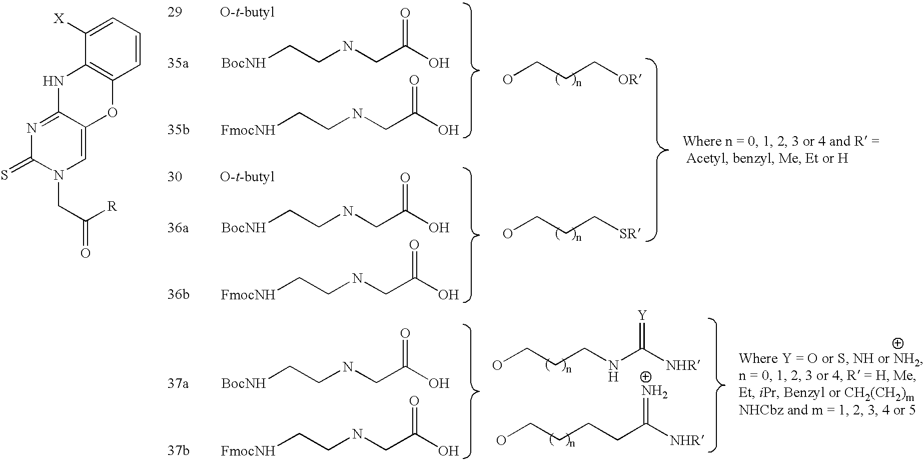

### Example 41

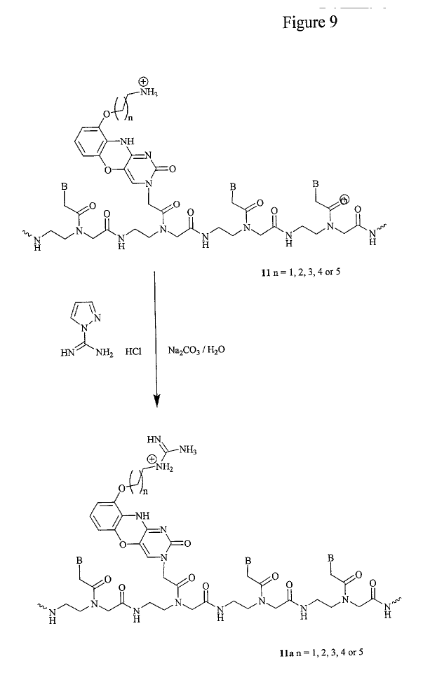

Post synthetic modification of PNA 11 (n=1). FIG. 9 shows preparation of PNA oligomer 11a. Referring to FIG. 9, a solution of PNA 11, 1H-pyrazole-1-carboxamide hydrochloride and Na₂CO₃ in water is stirred at ambient temperature for 4 B 6 h to obtain compound 11a (Bernatowics et. al., J. Org Chem. 1992, 57, 2497).

### Example 42

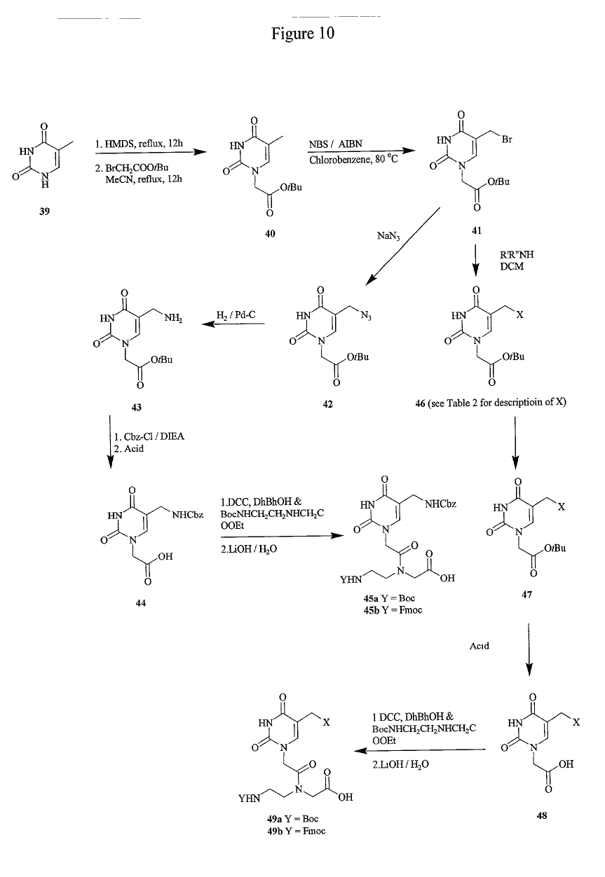

Synthesis of compound 40: FIG. 10 shows a representative synthesis of monocyclic compounds 49a and 49b. Referring to FIG. 10, compound 40 is synthesized from compound 39 as described in Example 1 for the synthesis of compound 2a.

### Example 43

Synthesis of compound 41: Compound 40 (1 mmol) and NBS (1.1 mmol) are suspended in chlorobenzene (10 mL) and the suspension is deoxygenated with argon for 30 min. The reaction mixture is heated to 80° C. under argon and AIBN (10 mol %) is added into the preheated solution (No et. el., Syn. Commun. 2000, 30, 3873). The reaction mixture is allowed to stir for 2 h by maintaining the temperature at 80° C. Filter off the solid residue and the filtrate is concentrated to dryness to obtain compound 41. Compound 41 is directly used for further experiments without purification.

### Example 44

Synthesis of compound 42: A suspension of compound 41 (1 mmol) and sodium azide (1.5 mmol) in anhydrous DMF (5 mL) is stirred at 120° C. for 2 h. After removing the solid residue by filtration, DMF is removed from the filtrate. Residue is taken in ethyl acetate and washes with water to remove dissolved sodium salt. Evaporation of the solvent follows purification to obtain the desired compound 42.

### Example 45

Synthesis of compound 43: Compound 42 is subjected to catalytic hydrogenation over Pd-C, as explained in Example 12, to obtain compound 43.

### Example 46

Synthesis of compound 44: Amino group of compound 43 is protected as Cbz by reacting 43 (1 mmol) with benzyl chloroformate (1.2 mmol) in presence of DIEA in dichloromethane (5 mL). The amine-protected compound thus obtained is treating with trifluoroacetic acid to obtain compound 44.

### Example 47

Synthesis of compound 45a: Standard PNA backbone, ethyl-N-(2-(t-butyloxycarbonylamino)ethyl)glycinate, is attached to compound 44 as described in Example 10 for the synthesis of compound 7a followed by alkaline hydrolysis to obtain compound 45a.

### Example 48

Synthesis of compound 45b: The Boc protected PNA monomer 45a is converted to the corresponding Fmoc protected monomer 45b as described in Example 12 for the synthesis of compound 9a.

### Example 49

Synthesis of compound 46 (X=NHCH₂CH₂NHCbz). Compound 41 (1 mmol) is stirred with benzyl N-(2-aminoethyl)carbamate (1.5 mmol) in presence of DIEA in dichloromethane (10 mL) to obtain compound 46.

### Example 50

Synthesis of compound 47 (X=N(Cbz)CH₂CH₂NHCbz). Secondary amino group of compound 46 is protected as Cbz as described in Example 18 for the synthesis of compound 13 to obtain compound 47.

### Example 51

Synthesis of compound 48 (X=N(Cbz)CH₂CH₂NHCbz). Acid hydrolysis of compound 47 yields compound 48.

### Example 52

Synthesis of compound 49a (X=N(Cbz)CH₂CH₂NHCbz). Standard PNA backbone, ethyl-N-(2-(t-butyloxycarbonylamino)ethyl)glycinate, is attached to compound 48 as described in Example 10 for the synthesis of compound 7a followed by hydrolysis of the ethyl ester under alakaline condition to obtain compound 49a.

### Example 53

Synthesis of compound 49b (X=N(Cbz)CH₂CH₂NHCbz). The Boc protected PNA monomer 49a is converted to the corresponding Fmoc protected monomer 49b as described in Example 12 for the synthesis of compound 9a.

### Example 54

Synthesis of compound 49d: Commercially available BocT PNA monomer is converted to its t-butyl ester (49c) and compound 49c is subjected to photolytic bromination as described in Example 43 to obtain compound 49d.

### Example 55

Synthesis of compound 49a (X=NH(Cbz), Y=Boc). Compound 49d is initially reacted with sodium azide as described in Example 44 to obtain the corresponding azide derivative which is then reduced to the corresponding amine (Example 45). The amine obtained is protected as benzyl carbamate as described in Example 18 to obtain compound 49a (as specified).

### Example 56

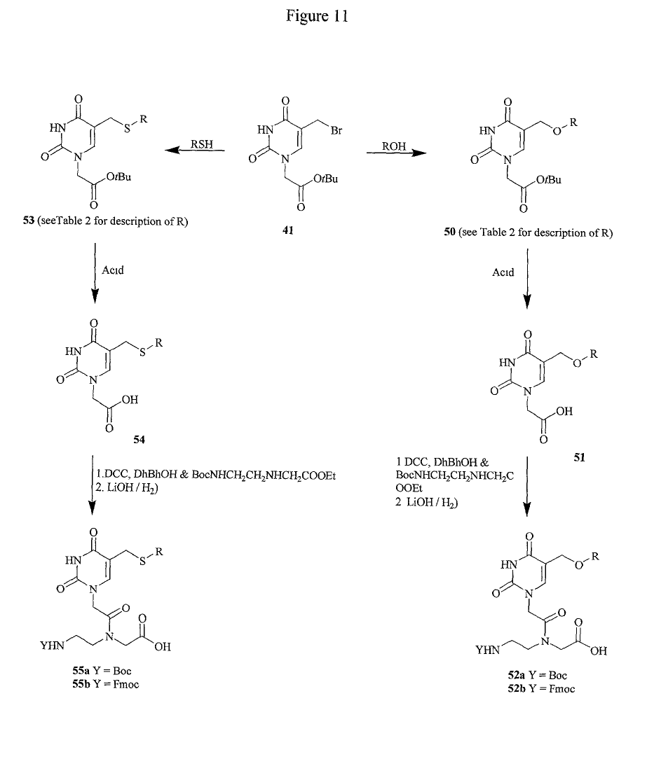

Synthesis of compound 50 (R=CH₂CH₂NHCbz). FIG. 11 shows a representative synthesis of monocyclic compounds 52a, 52b, 55a, and 55b. Referring to FIG. 11, compound 41 (1 mmol) is stirred with benzyl N-(2-hydroxyethyl)carbamate (2 mmol) and DIEA (1.5 mmol) in dichloromethane (10 mL) overnight to obtain compound 50.

### Example 57

Synthesis of compound 51 (R=CH₂CH₂NHCbz). Acid hydrolysis of compound 50 yields compound 51.

### Example 58

Synthesis of compound 52a (R=CH₂CH₂NHCbz). Boc protected PNA backbone, ethyl-N-(2-(t-butyloxycarbonylamino)ethyl)glycinate, is attached to compound 51 as described in Example 10 for the synthesis of compound 7a followed by hydrolysis of the ethyl ester under alkaline condition to obtain compound 52a.

### Example 59

Synthesis of compound 52b (R=CH₂CH₂NHCbz). The Boc protected PNA monomer 52a is converted to the corresponding Fmoc protected monomer 52b as described in Example 12 for the synthesis of compound 9a.

### Example 60

Synthesis of compound 53 (R=Et): Compound 41 (1 mmol) is stirred with ethanethiol (2 mmol) and DIEA (1.5 mmol) in dichloromethane (10 mL) overnight to obtain compound 54.

### Example 61

Synthesis of compound 54 (R=Et). Acid hydrolysis of compound 53 yields compound 54.

### Example 62

Synthesis of compound 55a (R=Et). Boc protected PNA backbone, ethyl-N-(2-(t-butyloxycarbonylamino)ethyl)glycinate, is attached to compound 54 as described in Example 10 for the synthesis of compound 7a followed by alkaline hydrolysis to obtain compound 55a.

### Example 63

Synthesis of compound 55b (R=Et). The Boc protected PNA monomer 55a is converted to the corresponding Fmoc protected monomer 55b as described in Example 12 for the synthesis of compound 9a.

Referring to the previous Examples, representative monocyclic compounds 49a, 49b, 52a, 52b, 55a, and 55b are shown below in Table 8

TABLE 8

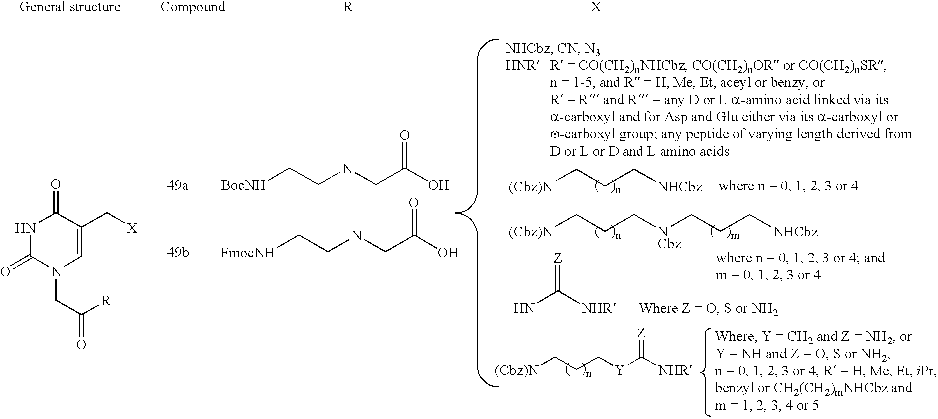

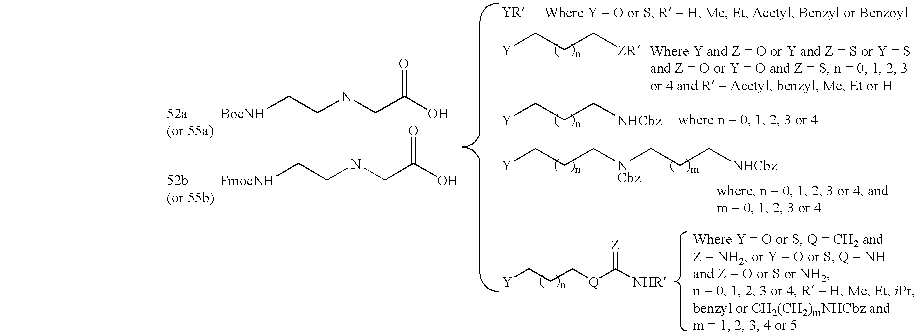

### Example 64

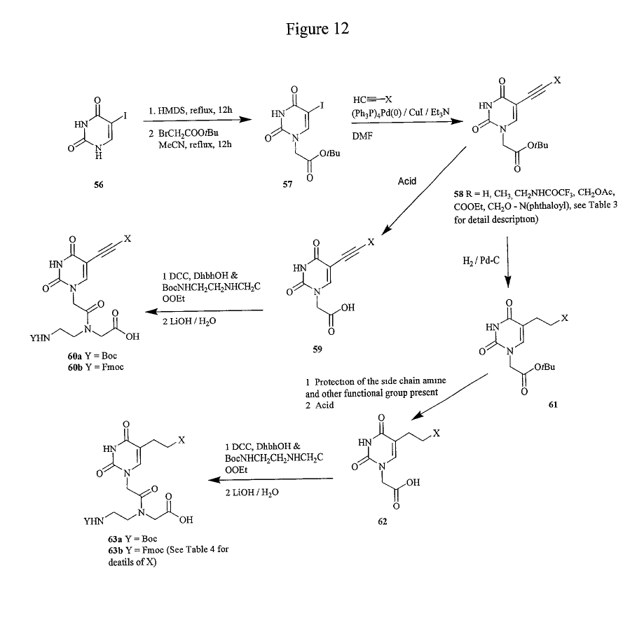

Synthesis of compound 57: FIG. 12 shows a representative synthesis of monocyclic compounds 63a and 63b. Referring to FIG. 12, compound 57 is prepared from 5-iodouracil (56) as described in Example 1 for the synthesis of compound 2a.

### Example 65

Synthesis of compound 58 (X=CH₂NHCOCF₃). A stirred solution of compound 57 (1 mmol) in anhydrous DMF (5 mL) is deoxygenated by bubbling argon for 30 min. (Ph₃P)₄Pd (0.1 mmol), CuI (0.2 mmol) are added into the soltuion and subsequently anhydrous TEA (2 mmol) and N-trifluoroacetyl propargylamine (3 mmol). The reaction mixture is allowed to stir for 24 h at ambient temperature under argon to obtain the desired compound 58 (Morvan et. al., Tetrahedron, 1998, 54, 71).

### Example 66

Synthesis of compound 59 (X=NHCOCF₃). Acid hydrolysis of compound 58 yields compound 59.

### Example 67

Synthesis of compound 60a (X=CH₂NHCbz). Compound 59 is stirring with methanolic ammonia to remove the trifluoroacetyl group and the free amine thus formed is protected as NHCbz using benzyl chloroformate as described in Example 18. After Cbz protection, Boc protected PNA backbone, ethyl-N-(2-(t-butyloxycarbonylamino)ethyl)glycinate, is attached to the carboxylic group as described in Example 10 for the synthesis of compound 7a followed by basic hydrolysis to obtain compound 60a.

### Example 68

Synthesis of compound 60b (X=CH₂NHCbz). The Boc protected PNA monomer 60a is converted to the corresponding Fmoc protected monomer 60b as described in Example 12 for the synthesis of compound 9a.

### Example 69

Synthesis of compound 58 (X=COOEt). Compound 58 (X=COOEt) is prepared under identical conditions for the preparation of compound 58 (X=CH₂NHCOCF₃) as described in Example 65 using ethyl propiolate instead of N-trifluoroacetyl propargylamine.

### Example 70

Synthesis of compound 59 (X=COOEt). Acid hydrolysis of compound 58 (X=COOEt) yields compound 59.

### Example 71

Synthesis of compound 60a (X=COOEt). Compound 59 (1 mmol) is coupled to allyl-N-(2-(t-butyloxycarbonylamino)ethyl)glycinate as described in Example 10 for the synthesis of compound 7a to obtain the corresponding allyl ester. The allyl ester in anhydrous THF is stirred with (Ph₃P)₄Pd (10 mol %) and anhydrous morpholine (10 molar eq.) for 30 min at ambient temperature to obtain compound 60a (Kunz and Waldmann, Angew. Chem. Int. Ed. Engl., 1984, 23, 71).

### Example 72

Synthesis of compound 60b (X=COOEt). The Boc protected PNA monomer 60a (R=COOEt) is converted to the corresponding Fmoc protected monomer 60b as described in Example 12 for the synthesis of compound 9a.

### Example 73

Synthesis of compound 61 (X=COOEt). Catalytic hydrogenation of compound 58 (X=COOEt) over Pd-C at a hydrogen pressure of 40 psi for 24 h yields compound 61.

### Example 74

Synthesis of compound 62 (X=COOEt). Acid hydrolysis of compound 61 yields compound 62.

### Example 75

Synthesis of compound 63a (X=COOEt). Compound 63a is obtained from compound 62 (X=COOEt) and allyl-N-(2-(t-butyloxycarbonylamino)ethyl)glycinate as described in Example 71.

### Example 76

Synthesis of compound 63b (X=COOEt). The Boc protected PNA monomer 63a (X=COOEt) is converted to the corresponding Fmoc protected monomer 63b as described in Example 12 for the synthesis of compound 9a.

Referring to the previous Examples, representative compounds 61, 63a, and 63b are described below in Table 9:

TABLE 9

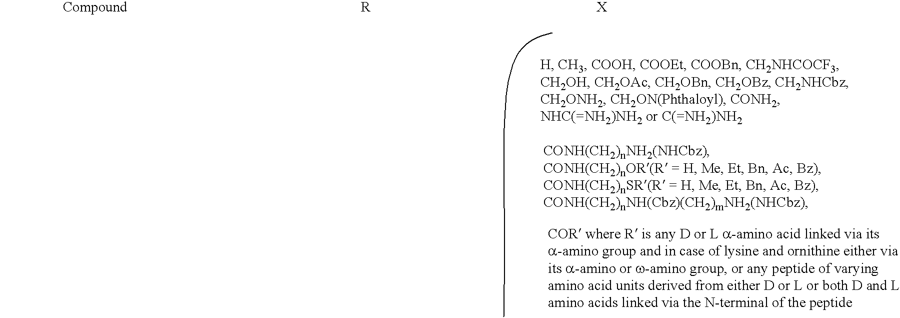

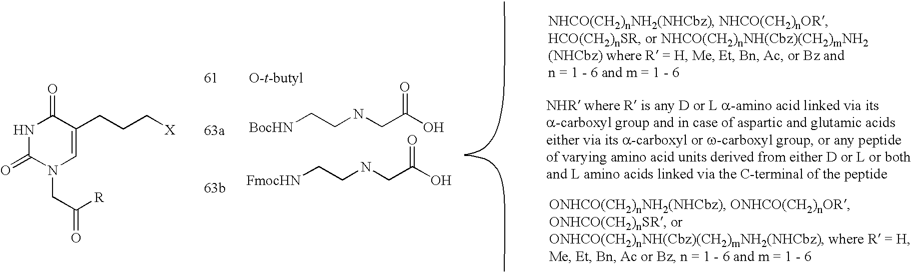

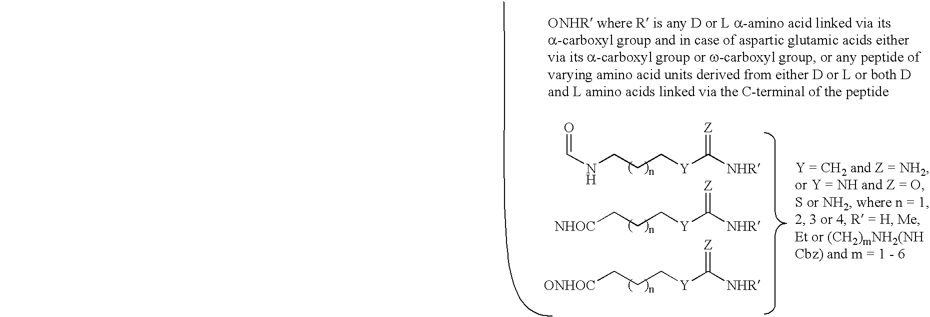

### Example 77

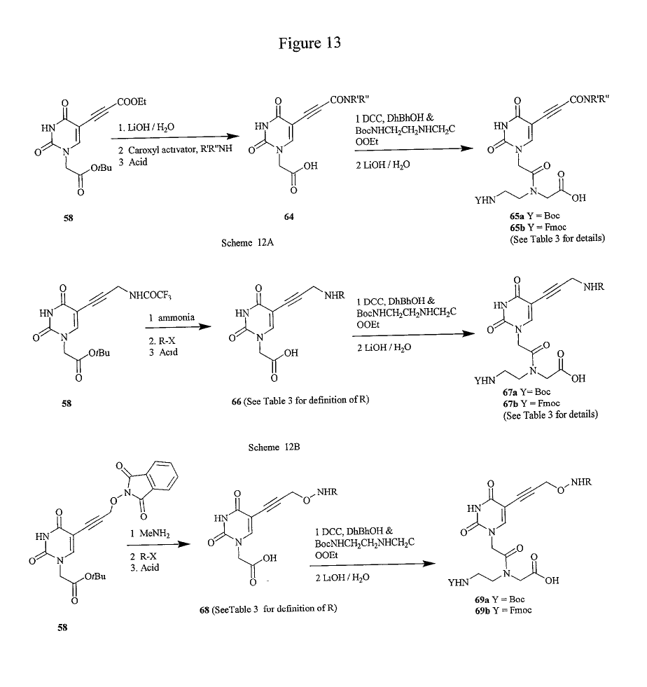

Synthesis of compound 64 (R′=H, R″=CH₂CH₂NHCbz). FIG. 13 shows a representative synthesis of monocyclic compounds 65a, 65b, 67a, 67b, 69a, and 69b. Referring to FIG. 13, ethyl ester of compound 58 (1 mmol) is hydrolyzed under basic condition as described in Example 11. The free carboxylic acid thus obtained is coupled to benzyl N-(2-aminoethyl)carbamate (1.1 mmol) in the presence of DCC and DMAP after which the t-butyl ester is removed under acidic condition to obtain compound 64.

### Example 78

Synthesis of compound 65a (R′=H, R″=CH₂CH₂NHCbz). Coupling of ethyl-N-(2-(t-butyloxycarbonylamino)ethyl)glycinate to the acid 64 as described in Example 10 followed by hydrolysis of the ester under basic condition yields compound 65a.

### Example 79

Synthesis of compound 65b (R′=H, R″=CH₂CH₂NHCbz). Compound 65b is obtained from compound 65a as described in Example 12 for the synthesis of compound 8a.

### Example 80

Synthesis of compound 66 (R=COCH₂CH₂NH(Cbz)). After removing the trifluoroacetyl protection of compound 58 by methanolic ammonia treatment, the resulting free amine is coupled to N-(carbobenzyloxy)—□-alanine under peptide coupling condition as described in Example 10. The product formed is treated with trifluoroacetic acid to obtain compound 66.

### Example 81

Synthesis of compound 67a (R=COCH₂CH₂NH(Cbz)). Acid 66 is coupled to ethyl-N-(2-(t-butyloxycarbonylamino)ethyl)glycinate as described in Example 10 for the synthesis of compound 7a. The ethyl ester formed is hydrolyzed under alkaline condition to obtain compound 67a.

### Example 82

Synthesis of compound 67b (R=COCH₂CH₂NH(Cbz)). The Fmoc PNA monomer 67b is obtained from 67a as described in Example 12.

### Example 83

Synthesis of compound 68 (R=COCH₂CH₂NH(Cbz)). Compound 58 upon treatment with methylamine undergoes N-phthaloyl deprotection. The hydroxylamine derivative thus formed is coupled to N-(carbobenzyloxy)-β-alanine under peptide coupling condition as described in Example 10. Acid hydrolysis of the ester formed yields compound 68.

### Example 84

Synthesis of compound 69a (R=COCH₂CH₂NH(Cbz)). Compound 69a is prepared from compound 68 under identical conditions as that of compound 67a (Example 81).

### Example 85

Synthesis of compound 69b (R=COCH₂CH₂NH(Cbz)). Compound 69b is prepared from compound 69a as described in Example

Referring to the previous Examples, representative monocyclic compounds 58, 60a, 60b, 65a, 65b, 67a, 67b, 69a, and 69b are shown below in Table 10

TABLE 10

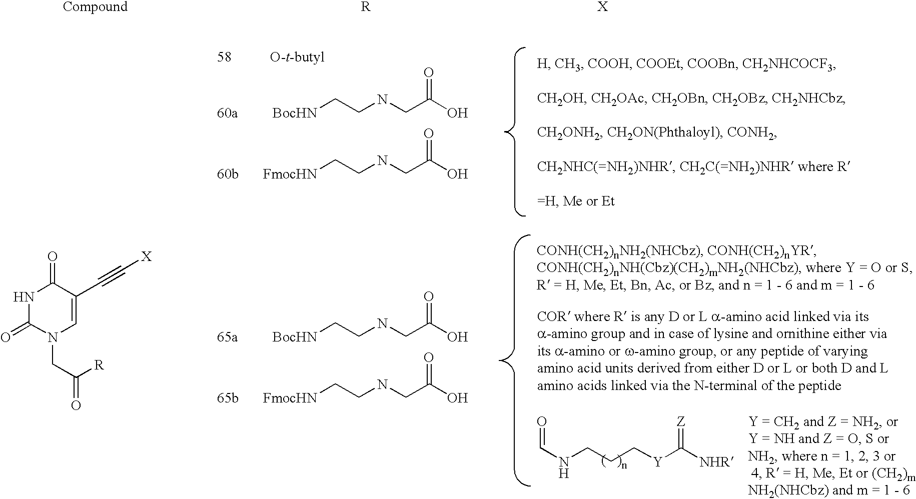

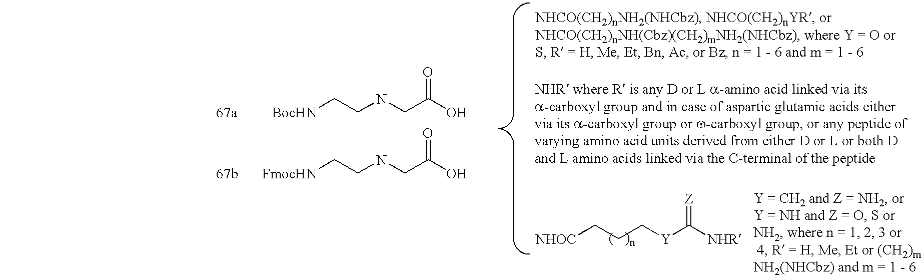

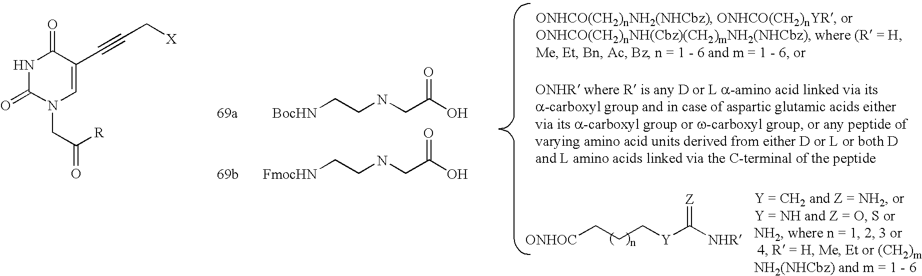

### Example 86

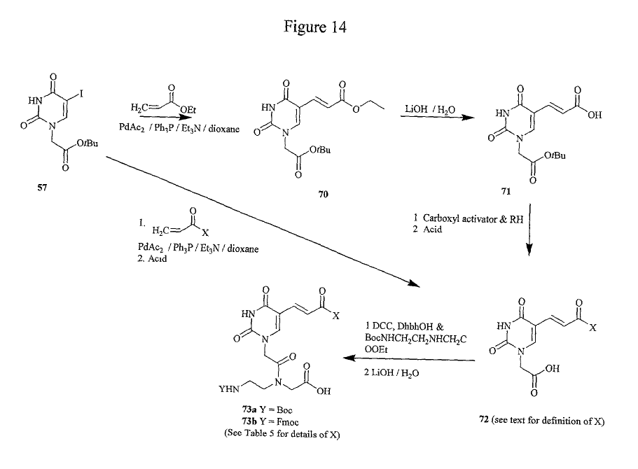

Synthesis of compound 70: FIG. 14 shows a representative synthesis of monocyclic compounds 73a and 73b. Referring to FIG. 14, reaction compound 57 with ethyl acrylate in presence of Ph₃P, Pd(II)acetate and TEA in dioxane under reflux yields compound 70 (Matulic-Adamic et. al., Bio. Med. Chem. Lett., 2000, 10, 1299).

### Example 87

Synthesis of compound 71: Hydrolysis of the ethyl ester 70 under basic condition yields compound 71.

### Example 88

Synthesis of compound 72 (X=NHCH₂CH₂NH(Cbz)). Coupling of compound 71 to benzyl N-(2-aminoethyl)carbamate under peptide coupling conditions as described in Example 10 followed by acid hydrolysis yields compound 72.

### Example 89

Synthesis of compound 73a (X=NHCH₂CH₂NH(Cbz)). Compound 73a is obtained from compound 72 as described in Example 10 for the synthesis of compound 7a.

### Example 90

Synthesis of compound 73b (X=NHCH₂CH₂NH(Cbz)). Compound 73b is prepared from compound 73a as described in Example 12.

Referring to the previous Examples, representative compounds 71, 73a, and 73b are shown below in Table 11:

TABLE 11

| | | |
|---|---|---|
| Compound | R | X |

| | |
|---|---|
| 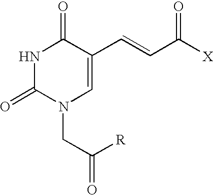 | 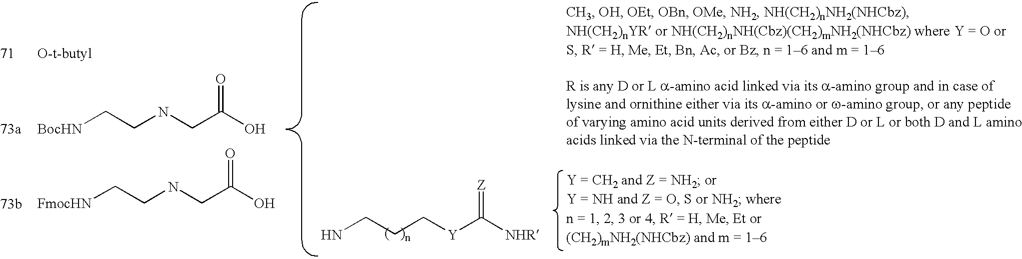 |

### Example 91

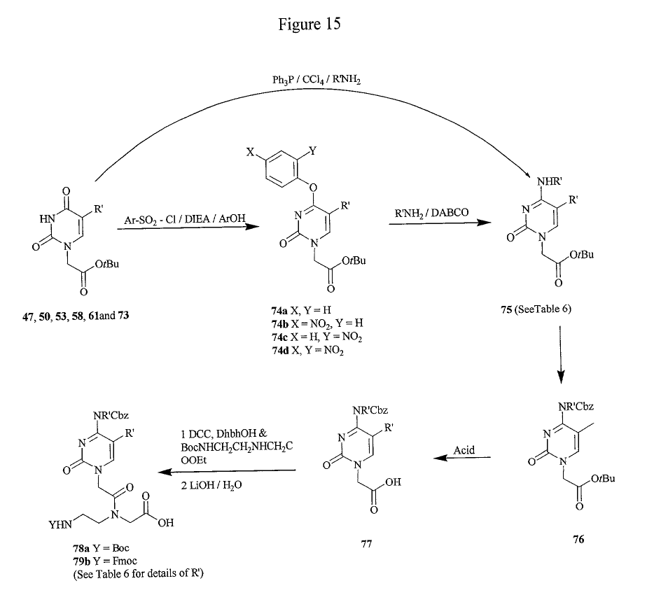

Synthesis of compound 74d (R′=CH₂ N(Cbz)CH₂CH₂NHCbz). FIG. 15 shows a representative synthesis of monocyclic compounds 78a and 78b. Referring to FIG. 15, compound 47 (X=N(Cbz)CH₂CH₂NHCbz) is stirred with 2-mesitylenesulfonyl chloride (2 molar eq.), DIEA (2 molar eq.) and DMAP (10 mol %) in anhydrous dichloromethane under argon at ambient temperature. After the complete conversion of compound 47 into the corresponding C4-O-sulfonate, 2,4-dinirtophenol (1.5 molar eq.) and DABCO (1,4-diazbicyclo[2,2,2]octane) are added into the reaction mixture and stirred for overnight to obtain compound 74d.

### Example 92

Synthesis of compound 75 (R′=CH₂ N(Cbz)CH₂CH₂NHCbz, R″=H). Compound 74d upon treatment with ammonia under pressure at 60° C. for 48 h yields compound 75.

### Example 93

Synthesis of compound 75 (R′=CH₂ N(Cbz)CH₂CH₂NHCbz, R″=H). Compound 47 (X=N(Cbz)CH₂CH₂NHCbz) is stirred with Ph₃P and CCl₄ in dichloromethane at ambient temperature for 2 h after which dry ammonia is bubbled through the reaction mixture to obtain compound 75 directly from compound 47.

### Example 94

Synthesis of compound 76 (R′32 CH₂ N(Cbz)CH₂CH₂NHCbz, R″=Cbz). Compound 75 is reacted with benzyl chloroformate in presence of DIEA to obtain compound 76.

### Example 95

Synthesis of compound 77 (R′=CH₂ N(Cbz)CH₂CH₂NHCbz, R″=Cbz). Acid hydrolysis of compound 76 yields compound 77.

### Example 96

Synthesis of compound 78a (R′=CH₂ N(Cbz)CH₂CH₂NHCbz, R″=Cbz). Compound 78a is prepared from compound 77 and ethyl-N-(2-(t-butyloxycarbonylamino)ethyl)glycinate as described in Example 10.

### Example 97

Synthesis of compound 78b R′=CH₂ N(Cbz)CH₂CH₂NHCbz, R″=Cbz). Compound 78b is prepared from compound 78a as described in Example 12.

Referring to the previous Examples, representative compounds 74, 78a, and 78b are shown below in Table 12:

TABLE 12

| | | |
|---|---|---|
| Compound | R | R′ |

| | |
|---|---|
| 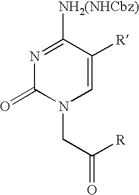 | 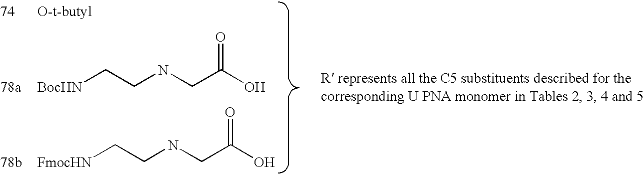 |

### Example 98

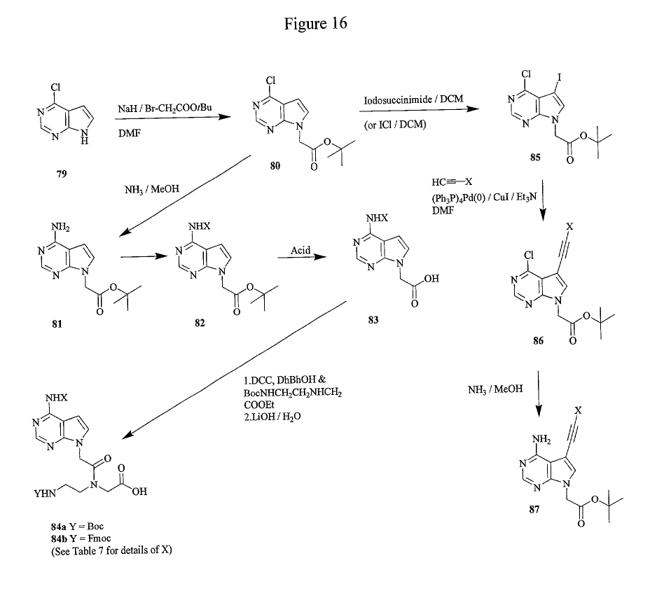

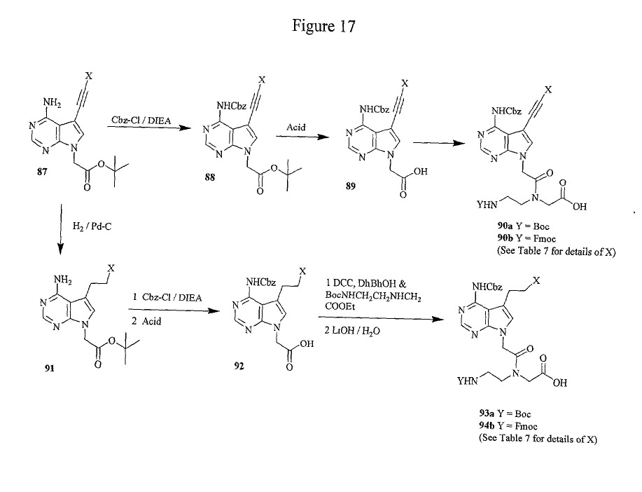

Synthesis of compound 80: FIG. 16 shows a representative synthesis of dicyclic compounds 84a, 84b, and 87. FIG. 17 shows a representative synthesis of dicyclic compounds 90a, 90b, 93a, and 94b. Referring to FIGS. 16 & 17, compound 79 (1 mmol) is added into a suspension of NaH (1.2 mmol) in anhydrous DMF at 0° C. under argon. Effervescence follows. After 10 min, t-butyl bromoacetate is added into the reaction at 0° C. and slowly bringing the reaction to room temperature. The stirring is continued for 4 h, excess NaH is quenched by methanol and standard works up follows to obtain compound 80.

### Example 99

Synthesis of compound 81: Compound 80 is treated with ammonia under pressure at elevated temperature to obtain compound 81.

### Example 100

Synthesis of compound 82 (X=Cbz). Compound 81 upon treatment with benzyl chlorofornate in presence of base yields compound 82.

### Example 101

Synthesis of compound 83 (X=Cbz). Acid hydrolysis of compound 82 yields compound 83

### Example 102

Synthesis of compound 84a (X=Cbz). Compound 84a is synthesized from compound 83 and ethyl-N-(2-(t-butyloxycarbonylamino)ethyl)glycinate as described in Example 10 followed by alkaline hydrolysis.

### Example 103

Synthesis of compound 84b (X=Cbz). Compound 84b is prepared from compound 84a as described in Example 12.

### Example 104

Synthesis of compound 85: A solution of compound 80 (1 mmol) and N-iodosuccinimide (1 mmol) in anhydrous DMF (5 mL) is stirred at ambient temperature for overnight to obtain compound 85 (Balow et. al., Nucleic Acids Res., 1998, 26, 3350).

### Example 105

Synthesis of compound 86 (X=Me). Compound 86 is prepared from compound 85 and propyne under the conditions as described in Example 65 for the preparation of compound 58.

### Example 106

Synthesis of compound 87 (X=Me). Compound upon treatment with ammonia at elevated temperature yields compound 87.

### Example 107

Synthesis of compound 90a (X=Me). Compound 90a is prepared from compound 87 as described in Examples 100, 101 and 102.

### Example 108

Synthesis of compound 90b (X=Me). Compound 90b is prepared from compound 90a as described in Example 12.

Referring to the previous Examples, representative compounds 81, 84a, 84b, 88, 90a, 90b are shown below in Table 13:

TABLE 13

| | | | |
|---|---|---|---|
| General structure | Compound | R | X |

| | |
|---|---|
| 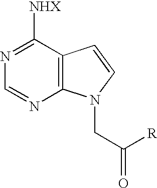 | 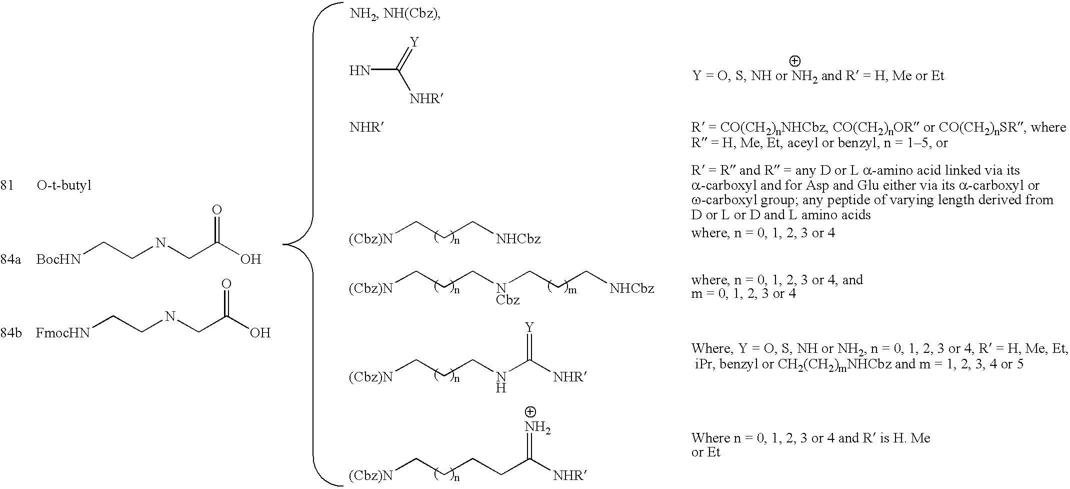 |
| 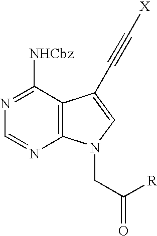 | 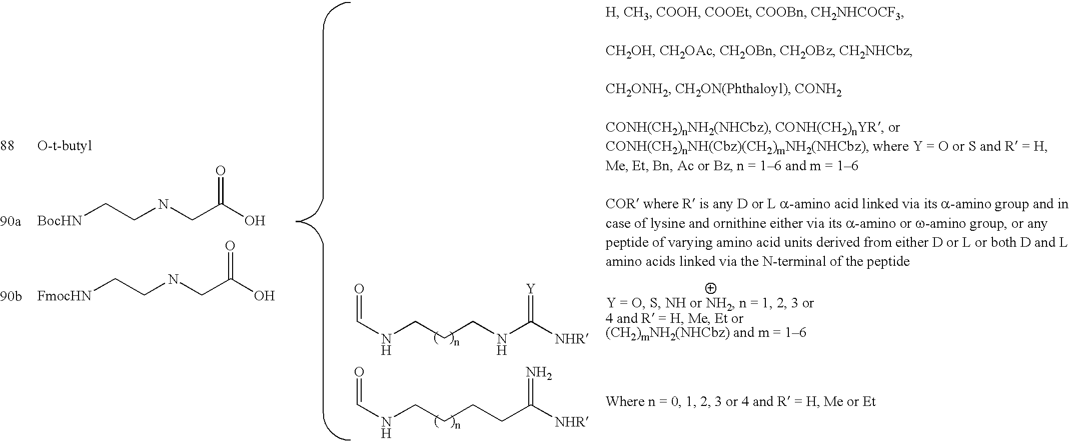 |

### Example 109

Synthesis of compound 91 (X=Me). Catalytic hydrogenation of compound 87 over Pd-C as described in Example 73 yields compound 91.

### Example 110

Synthesis of compound 93a (X=Me). Compound 93a is prepared from compound 91 as described in Examples 100, 101 and 102.

### Example 111

Synthesis of compound 93b (X=Me). Compound 93b is prepared from compound 93a as described in Example 12 for the preparation of compound 9a.

Referring to the previous Examples, representative compounds 88, 90a, 90b, 91, 93a, and 93b are shown below in Table 14:

TABLE 14

| | | | |
|---|---|---|---|
| General structure | Compound | R | X |

| | |
|---|---|
| 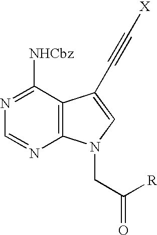 | 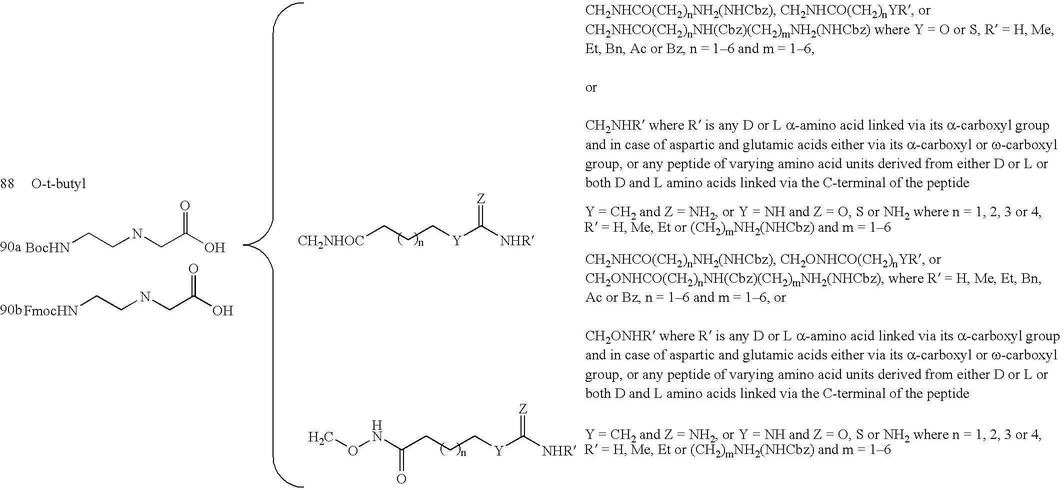 |
| 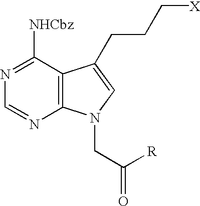 | 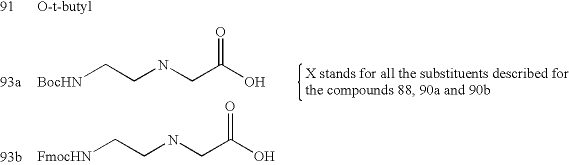 |

### Example 112

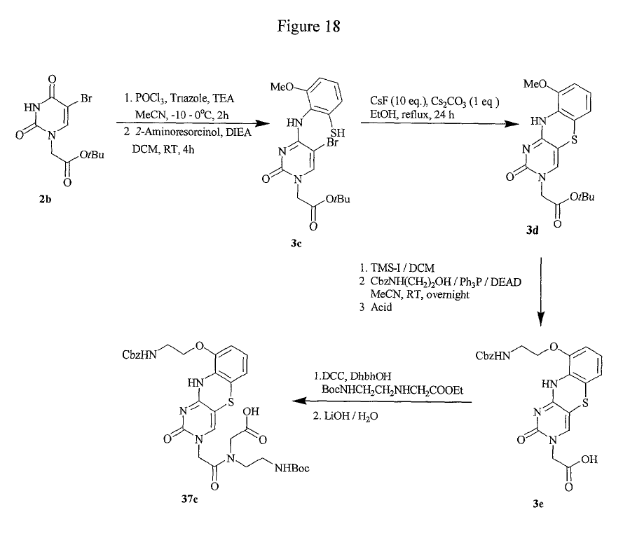

Synthesis of compound 3c: FIG. 18 shows a representative synthesis of tricyclic compound 37c. Referring to FIG. 18, compound 3c is prepared from compound 2b and 2-amino-3-methoxy-benzenethiol (Agrawal et. al., Heterocycle. Commun., 1998, 4, 589) with compound 2b as described in Example 3 for the synthesis of compound 3a.

### Example 113

Synthesis of compound 3d: A suspension of compound 3c (1 mmol), CsF (10 mmol) and Cs₂CO₃ (1 eq.) are refluxed in absolute ethanol as described in Example 7 for the synthesis of compound 5a to get compound 3d.

### Example 114

Synthesis of compound 3e: After thorough drying compound 3d (1 mmol) is treated with TMS-I (1 mmol) in dichloromethane. After 5 min, solvent and methyl iodide are removed under vacuum. Residue is redissolved in dichloromethane, washes with bicarbonate. The residue after thorough drying is reacted with benzyl N-(2-hydroxyethyl)carbamate as described in Example 5 to obtain the corresponding O-alkylated product. Hydrolysis of the t-butyl ester formed under acidic condition yields the desired product 3e.

### Example 115

Synthesis of compound 37c: Compound 37c is prepared by alkaline hydrolysis of the product obtained from DCC and DhbhOH mediated coupling of compound 3e (1 mmol) to ethyl-N-(2-(t-butyloxycarbonylamino)ethyl)glycinate (1 mmol) as described in Example 10.

Referring to Example 115, representative tricyclic compounds are disclosed in Table 15:

TABLE 15

| | | |
|---|---|---|
| Compound | R | X |

| | |
|---|---|
| 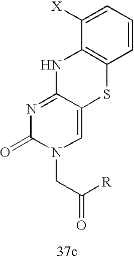 | 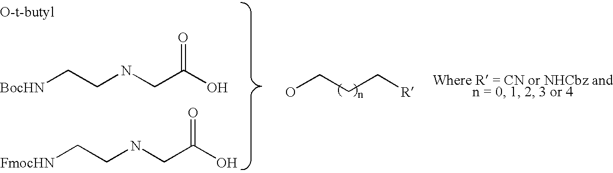 |
| | 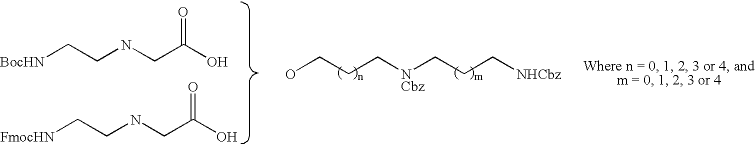 |
| | 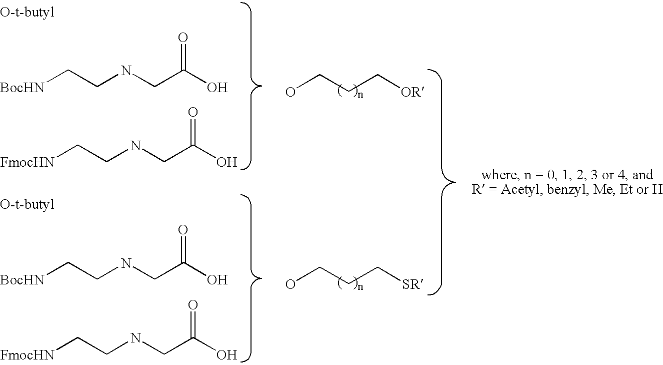 |
| | 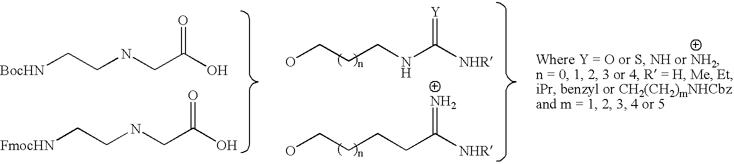 |

Other representative compounds of the present invention include tricyclic compounds as shown below in Table 16:

TABLE 16

| | | |
|---|---|---|
| Compound | R | X |

| | |
|---|---|
| 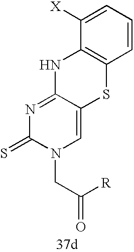 | 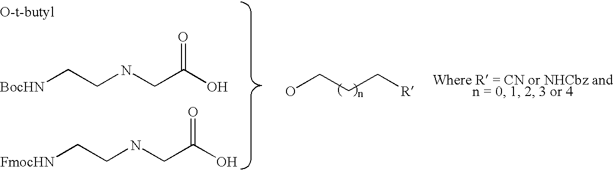 |
| | 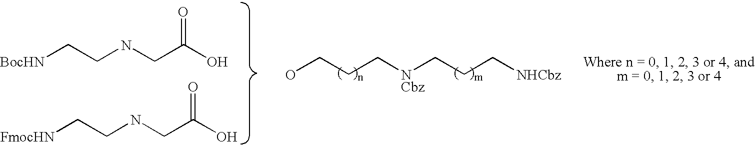 |
| | 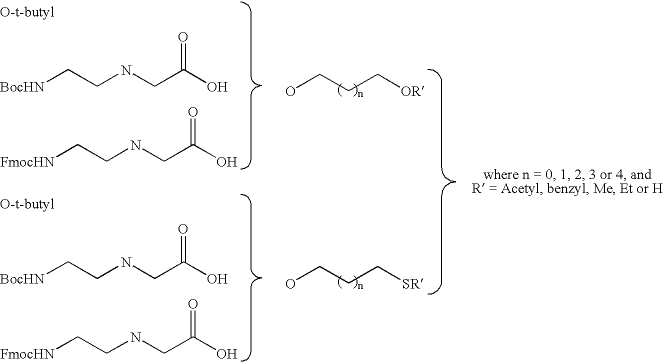 |
| | 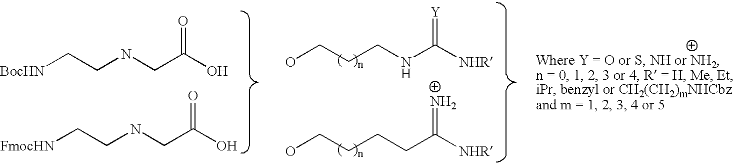 |

### Example 116

Synthesis of compound 94a-d monomer: Compound 5 (n=0, R=NHCbz, Example 16) is coupled to four stereo isomers of the modified backbone derived from naturally occurring 4R-hydroxy-2S-proline as reported by Gangamani et. al. (Tetrahedron, 1996, 52, 15017) to obtain the four stereo isomers 94a-d.

### Example 117

Synthesis of compound 101b: Alkylation of the ‘G-clamp’ base at N1 under Mitsunobu alkylation condition using (3S, 5R)-5-t-butoxycarbonylaminomethyl-3-hydroxy-N-methoxycarbonylmethyl-2-pyrrolidinone (prepared according Puschl et. al., J. Org. Chem., 2001, 66, 707) gives the completely protected modified PNA monomer with appropriate stereochemistry. Alkaline hydrolysis of the methyl ester gives the desired monomer ready to use for the synthesis of PNA incorporated with the modified base 101b. The ‘G-clamp’ base is prepared by removal of 2′-deoxy sugar from the corresponding ‘G-clamp’ nucleoside (Lin and Matteucci, J. Am. Chem. Soc., 1998, 120, 8531) under acidic condition.

What is claimed is:

1. An oligomeric compound of formula I: 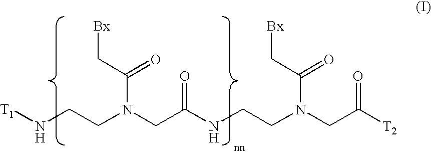
wherein:
T₁ is hydrogen, an amino protecting group, —C(O)R₅, substituted or unsubstituted C₁-C₁₀ alkyl, substituted or unsubstituted C₂-C₁₀ alkenyl, substituted or unsubstituted C₂-C₁₀ alkynyl, alkylsulfonyl, arylsulfonyl, a chemical functional group, a reporter group, a conjugate group, a D or L α-amino acid linked via the (α-carboxyl group or optionally through the ω-carboxyl group when the amino acid is aspartic acid or glutamic acid or a peptide derived from D, L or mixed D and L amino acids linked through a carboxyl group, wherein the substituent groups are selected from hydroxyl, amino, alkoxy, carboxy, benzyl, phenyl, nitro, thiol, thioalkoxy, halogen, alkyl, aryl, alkenyl and alkynyl;
T₂ is —OH, —N(Z₁)Z₂, R₅, D or L α-amino acid linked via the α-amino group or optionally through the ω-amino group when the amino acid is lysine or ornithine or a peptide derived from D, L or mixed D and L amino acids linked through an amino group, a chemical functional group, a reporter group or a conjugate group;
nn is from 2 to about 50;
each Bx is, independently, an optionally protected heterocyclic base moiety wherein at least one of said heterocyclic base moieties has one of formulas II or III: 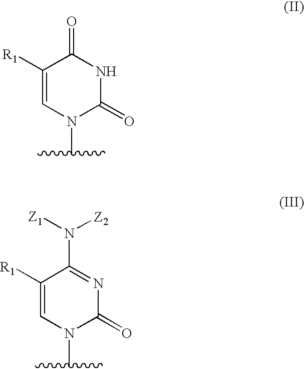
wherein:
R₁ is —CH₂—Q₂, —C≡C—Q₂, —CH₂—(CH₂)ₙ—Q₃, or —CH═CH—C(═O)—Q₄;
Q₁ is —N₃, —CN, —N(Z₁)Z₂, —N(Z₁)—(CH₂)ₙ—C(═NH₂)—N(H)—Z₃, —N(Z₁)—C(═J)—N(H)—Z₅, —L—(CH₂)ₙ—C(═O)Z₃, —L—(CH₂)ₙ—L—Z₃, —L—(CH₂)ₙ—N(H)Z₁—L—(CH₂)ₙ—N(Z₁)—(CH₂)ₙ—N(H)Z₁, —L—(CH₂)ₙ—C(═NH)N(Z₁)Z₃ or —L—(CH₂)ₙ—N(Z₁)—C(═J)—N(H)Z₃;
Q₂ is H, C₁-C₆ alkyl, —C(═O)—N(H)Z₁, —C(═O)—O—CH₂—CH₃, C(═O)—O—benzyl, —C(═O)—Z₄, —CH₂—O—Q₆, —CH₂—C(═NH)—N(H)—Z₃, —CH₂—N(H)—Z₂, —CH₂—N(H)—C(═O)—CF₃, —CH₂—N(H)Z₁, —CH₂—N(H)—C(═NH)—N(H)—Z₃ —CH₂—O—N(H)—C(═O)—(CH₂)ₙ—Q₅, —C(═O)—N(H)—(CH₂)ₙ—Q₅ or —CH₂—N(H)—C(═O)—(CH₂)ₙ—Q₅;
Q₃ is hydrogen, —O—C₁-C₆ alkyl, —N(H)—Z₁, —N(H)—Z₂, —N(H)—C(═O)—CF₃, —N(H)—C(═NH)—N(H)Z₁, —O—Q₆, —N(H)—C(═O)—(CH₂)ₙ—Q₅, —O—N(H)—C(═O)—(CH₂)ₙ—Q₅, —C(═O)—N(H)—(CH₂)ₙ—Q₅ or —C(═O)—Q₇;
Q₄is is Z₄, —L—Z₃, —N(H)Z₃, —C(═NH)—N(H)Z₃, —N(H)—C(═J) N(H)Z₃, N(Z₁)—(CH₂)ₙ—N(H)Z₃, —C(═O)—N(H)—(CH₂)ₙ—C(═NH)—N(H)Z₃, —C(═O)—N(H)—(CH₂)ₙ—N(H)—C(═J)—N(H)Z₃ or —C(═O)—N(H)—(CH₂)ₙ—N(Z₁)—(CH₂)ₙ—N(H)Z₁;
Q₅is —L—Z₃, —N(H)Z₃, —C(═NH)—N(H)Z₃, —N(H)—C(═J) N(H)Z₃ or N(Z₁)—(CH₂)ₙ—N(H)Z₃;
Q₆ is hydrogen, —N(H)Z₁, —N(H)Z₂, benzyl, benzoyl, —C(═O)—(CH₂)ₙ—H or phthalimido;
Q₇ is —OH, —O—C₁-C₆ alkyl, —O-benzyl, —Z₄, —N(H)Z₁,
each L is O or S;
each J is O, S or NH;
each n is from 1 to 6;
Z₁ is hydrogen, C₁-C₆ alkyl, or an amino protecting group;
Z₂ is hydrogen, C₁-C₆ alkyl, an amino protecting group, —C(═O)—(CH₂)ₙ—J—Z₃, a D or L α-amino acid linked via the α-carboxyl group or optionally through the ω-carboxyl group when the amino acid is aspartic acid or glutamic acid or a peptide derived from D, L or mixed D and L amino acids linked through a carboxyl group;
Z₃ is hydrogen, an amino protecting group, —C₁-C₆ alkyl, —C(═O)—CH₃, benzyl, benzoyl, or —(CH₂)ₙ—N(H)Z₁;
Z₄ is a D or L α-amino acid linked via the α-amino group or optionally through the ω-amino group when the amino acid is lysine or ornithine or a peptide derived from D, L or mixed D and L amino acids linked through an amino group;
Z₅ is hydrogen, an amino protecting group or —C(═O)—(CH₂)ₙ—J—Z₃; and
each R₅ is a carbonyl protecting group.

2. The oligomeric compound of claim 1 wherein R₁ is —CH₂—Q₁.

3. The oligomeric compound of claim 2 wherein Q₁ is —N₃, —CN, —N(Z₁)Z₂, —N(Z₁)—(CH₂)ₙ—C(═NH)—N(H)—Z₃, —N(Z₁)—C(═J)—N(H)—Z₅, —L—(CH₂)ₙ—C(═O)Z₃, —L—(CH₂)ₙ—L—Z₃, —L—(CH₂)ₙ—N(H)Z₁, —L—(CH₂),—N(Z₁)—(CH₂)ₙ—N(H)Z₁, —L—(CH₂)ₙ—C(═NH)N(Z₁)Z₃ or —L—(CH₂)ₙ—N(Z₁)—C(═J)—N(H)Z₃.

4. The oligomeric compound of claim 1 wherein Z₁, Z₂, Z₃, Z₄ and Z₅ are each independently hydrogen, methyl or an amino protecting group,

5. The oligomeric compound of claim 1 wherein each n is independently from 1 to about 3.

6. The oligomeric compound of claim 1 wherein R₁ is —C≡—C—Q₂.

7. The oligomeric compound of claim 6 wherein Q₂ is H, methyl, ethyl, —C(═O)—N(H)Z₁, —CH₂—N(H)—Z₂ or —CH₂—N(H)—C(═NH)—N(H)—Z₅.

8. The oligomeric compound of claim 1 wherein R₁ is —CH₂—(CH₂)ₙ—Q₃.

9. The oligomeric compound of claim 8 wherein each Q₃ is hydrogen, —O—CH₃, —O—CH₂CH₃, —N(H)—Z₁, —N(H)—Z₂, —N(H)—C(═O)—CF₃ or —N(H)—C(═NH)—N(H)Z₁.

10. The oligomeric compound of claim 8 wherein Q₃ is —N(H)—C(═O)—(CH₂)ₙ—Q₅, —O—N(H)—C(═O)—(CH₂)ₙ—Q₅ or —C(═O)—N(H)—(CH₂)ₙ—Q₅ and Q₅is —N(H)Z₃, —C(═NH)—N(H)Z₃ or —N(H)—C(═J) N(H)Z₃.

11. The oligomeric compound of claim 8 wherein Q₃ is —O—Q₆ and Q₆ is hydrogen, —N(H)Z₁ or —N(H)Z₂.

12. The oligomeric compound of claim 1 wherein each R₁ is —CH═CH—C(═O)—Q₄.

13. The oligomeric compound of claim 12 wherein Q₄ is —OH, —N(H)Z₃, —C₁-C₆ alkyl, —O—C₁-C₆ alkyl, —O-benzyl or —N(H)—(CH₂)ₙ—Q₅.

14. The oligomeric compound of claim 13 wherein Q₄ is —N(H)Z₃ and Z₃ is Hydrogen or C₁-C₆ alkyl.

15. The oligomeric compound of claim 1 wherein each carbonyl protecting group is, independently, substituted or unsubstituted C₁-C₁₀ alkyl, trifluoromethyl, cyanoethyloxy, methoxy, ethoxy, t-butoxy, allyloxy, 9-fluorenylmethoxy, 2-(trimethylsilyl)-ethoxy, 2,2,2-trichloroethoxy, benzyloxy, butyryl, iso-butyryl, phenyl or aryl.

16. The oligomeric compound of claim 1 wherein T₁ is hydrogen, an amino protecting group, a reporter group or a D or L amino acid or a peptide.

17. The oligomeric compound of claim 16 wherein said D or L amino acid is lysine or glutamic acid.

18. The oligomeric compound of claim 1 wherein T₂ is —OH, —N(Z₁)Z₂, R₅ or a D or L amino acid or a peptide.

19. The oligomeric compound of claim 1 wherein said conjugate group is a contrast reagent, a cleaving agent, a cell targeting agent, polyethylene glycol, cholesterol, phospholipid, biotin, phenanthroline, phenazine, phenanthridine, anthraquinone, acridine, fluorescein, rhodamine, coumarin, pyrene, retinal or a cyanine dye.

20. The oligomeric compound of claim 1 wherein each Bx is independently selected from the group consisting of a radical of formula II, formula III, adeninyl, guaninyl, thyminyl, cytosinyl, uracilyl, 5-methylcytosinyl (5-me-C), 5-hydroxymethyl cytosinyl, xanthinyl, hypoxanthinyl, 2-aminoadeninyl, alkyl derivatives of adeninyl and guaninyl, 2-thiouracilyl, 2-thiothyminyl, 2-thiocytosinyl, 5-halouracilyl, 5-halocytosinyl, 5-propynyl uracilyl, 5-propynyl cytosinyl, 6-azo uracilyl, 6-azo cytosinyl, 6-azo thyminyl, 5-uracilyl (pseudouracil), 4-thiouracilyl, 8-substituted adeninyls and guaninyls, 5-substituted uracilyls and cytosinyls, 7-methylguaninyl, 7-methyladeninyl, 8-azaguaninyl, 8-azaadeninyl, 7-deazaguaninyl, 7-deazaadeninyl, 3-deazaguaninyl and 3-deazaadeninyl.

21. The oligomeric compound of claim 1 wherein nn is from about 8 to about 30.

22. The oligomeric compound of claim 1 wherein nn is from about 15 to about 25.

23. An oligomeric compound of formula I wherein:
T₁ is hydrogen, an amino protecting group, —C(O)R₅, substituted or unsubstituted C₁-C₁₀ alkyl, substituted or unsubstituted C₂-C₁₀ alkenyl, substituted or unsubstituted C₂-C₁₀ alkynyl, alkylsulfonyl, arylsulfonyl, a chemical functional group, a reporter group, a conjugate group, a D or L α-amino acid linked via the α-carboxyl group or optionally through the ω-carboxyl group when the amino acid is aspartic acid or glutamic acid or a peptide derived from D, L or mixed D and L amino acids linked through a carboxyl group, wherein the substituent groups are selected from hydroxyl, amino, alkoxy, carboxy, benzyl, phenyl, nitro, thiol, thioalkoxy, halogen, alkyl, aryl, alkenyl and alkynyl;
T₂ is —OH, —N(Z₁)Z₂, R₅, D or L α-amino acid linked via the α-amino group or optionally through the ω-amino group when the amino acid is lysine or ornithine or a peptide derived from D, L or mixed D and L amino acids linked through an amino group, a chemical functional group, a reporter group or a conjugate group;
nn is from 2 to about 50;
each Bx is, independently, an optionally protected heterocyclic base moiety; wherein at least one of said heterocyclic base moieties has one of formulas V or VI: 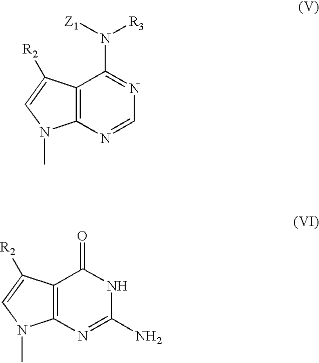
wherein:
R₂ is hydrogen and R₃ is Z₁, —C(═J)—N(H)Z₁, —C(═O)—(CH₂)ₙ—N(H)Z₁, —C(═O)—(CH₂)ₙ—L—Z₉, —(CH₂)ₙ—N(H)Z₁, —(CH₂)ₙ—N(Z₁)—(CH₂)ₙ—N(H)Z₁, —(CH₂)ₙ—N(H)—C(═J)—N(H)Z₃ or —(CH₂)ₙ—C(═NH)—N(H)Z₃;
or R₃ is hydrogen and R₂ is —C≡C—R₄ or —(CH₂)ₘ—R_{4;}
L is O or S;
J is O, S or NH;
m is from 2 to 6;
each n is from 1 to 6;
R₄ is H, C₁-C₆ alkyl, —CH₂OH, —CH₂—O—Q₆, —CH₂—N(H)—C(═O)—CF₃, —CH₂—N(H)Z₁, —CH₂—N(H)Z₂, —C(═O)—Z₄, —C(═O)—N(H)—(CH₂)ₙ—Q₅, —CH₂—N(H)—C(═O)—(CH₂)ₙQ₅ or —CH₂—O—N(H)—C(═O)—(CH₂)ₙ—Q₅;
Q₅is —L—Z₃, —N(H)Z₁, —C(═NH)—N(H)Z₃, —N(H)—C(═J) N(H)Z₃ or —N(Z₁)—(CH₂)ₙ—N(H)Z₁;
Q₆ is —N(H)Z₁, —N(H)Z₂, benzyl, benzoyl, —C(═O)—(CH₂)ₙ—CH₃ or phthalimido;
Z₁ is hydrogen, C₁-C₆ alkyl, or an amino protecting group;
Z₂ is hydrogen, C₁-C₆ alkyl, an amino protecting group, —C(═O)—(CH₂)ₙ—J—Z₃, a D or L α-amino acid linked via the α-carboxyl group or optionally through the ω-carboxyl group when the amino acid is aspartic acid or glutamic acid or a peptide derived from D, L or mixed D and L amino acids linked through a carboxyl group;
Z₃ is hydrogen, an amino protecting group, —C₁-C₆ alkyl, —C(═O)—CH₃, benzyl, benzoyl, or —(CH₂)ₙ—N(H)Z₁;
Z₄ is —OH, C₁-C₆ alkyl, benzyl, —N(H)Z₁, a D or L α-amino acid linked via the α-amino group or optionally through the ω-amino group when the amino acid is lysine or ornithing or a peptide derived from D, L or mixed D and L amino acids linked through an amino group;
Z₉ is hydrogen, —C₁-C₆ alkyl, —C(═O)—CH₃, benzyl or a D or L α-amino acid linked via the α-carboxyl group or optionally through the ω-carboxyl group when the amino acid is aspartic acid or glutamic acid or a peptide derived from D, L or mixed D and L amino acids linked through a carboxyl group; and
each R₅ is a carbonyl protecting group.

24. The oligomeric compound of claim 23 wherein R₂ is hydrogen and R₃ is Z₁, —C(═J)—N(H)Z₁ or —(CH₂)ₙ—C(═NH)—N(H)Z₃.

25. The oligomeric compound of claim 23 wherein R₃ is hydrogen and R₂ is —C≡C—R₄ or —(CH₂)ₘR₄.

26. The oligomeric compound of claim 25 wherein R₄ is H, C₁-C₃ alkyl, —CH₂OH, —CH₂—O—Q₆, —CH₂—N(H)Z₂ or —C(═O)—Z₄.

27. The oligomeric compound of claim 25 wherein R₄ is —C(═O)—Z₄, —C(═O)—N(H)—(CH₂)ₙ—Q₅, —CH₂—N(H)—C(═O)—(CH₂)ₙQ₅ or —CH₂—O—N(H)—C(═O)—(CH₂)ₙ—Q₅ and Q₅ is —N(H)Z₁ or —C(═NH)—N(H)Z₃.

28. The oligomeric compound of claim 25 wherein R₄ is —CH₂—O—Q₆ and Q₆ is —N(H)Z₂, —C(═O)—(CH₂)ₙ—CH₃ or phthalimido.

29. The oligomeric compound of claim 23 wherein T₂ is —N(Z₁)Z₂ and Z₂ is hydrogen, C₁-C₃ alkyl, an amino protecting group.

30. The oligomeric compound of claim 23 wherein R₃ is —(CH₂)ₙ—N(H)—C(═J)—N(H)Z₃ or —(CH₂)ₙ—C(═NH)—N(H)Z₃ and Z₃ is hydrogen, an amino protecting group, —C₁-C₃ alkyl or —C(═O)—CH₃.

31. The oligomeric compound of claim 25 wherein R₄ is —C(═O)—Z₄ and Z₄ is —OH, C₁-C₃ alkyl, benzyl or —N(H)Z₁.

32. The oligomeric compound of claim 23 wherein R₂ is —C(═O)—(CH₂)ₙ—L—Z₉ and Z₉ is hydrogen, —C₁-C₃ alkyl or —C(═O)—CH₃.

33. The oligomeric compound of claim 23 wherein each carbonyl protecting group is, independently, substituted or unsubstituted C₁-C₁₀ alkyl, trifluoromethyl, cyanoethyloxy, methoxy, ethoxy, t-butoxy, allyloxy, 9-fluorenylmethoxy, 2-(trimethylsilyl)-ethoxy, 2,2,2-trichloroethoxy, benzyloxy, butyryl, iso-butyryl, phenyl or aryl.

34. The oligomeric compound of claim 23 wherein T₁ is hydrogen, an amino protecting group, a reporter group, a D or L amino acid or a peptide.

35. The oligomeric compound of claim 34 wherein said D or L amino acid is lysine or glutamic acid.

36. The oligomeric compound of claim 23 wherein T₂ is —OH, —(Z₁)Z₂, R₅, a D or L amino acid or a peptide.

37. The oligomeric compound of claim 23 wherein each Bx is independently selected from the group consisting of a radical of formula V, formula VI, adeninyl, guaninyl, thyminyl, cytosinyl, uracilyl, 5-methylcytosinyl (5-me-C), 5-hydroxymethyl cytosinyl, xanthinyl, hypoxanthinyl, 2-aminoadeninyl, alkyl derivatives of adeninyl and guaninyl, 2-thiouracilyl, 2-thiothyminyl, 2-thiocytosinyl, 5-halouracilyl, 5-halocytosinyl, 5-propynyl uracilyl, 5-propynyl cytosinyl, 6-azo uracilyl, 6-azo cytosinyl, 6-azo thyminyl, 5-uracilyl (pseudouracil), 4-thiouracilyl, 8-substituted adeninyls and guaninyls, 5-substituted uracilyls and cytosinyls, 7-methylguaninyl, 7-methyladeninyl, 8-azaguaninyl, 8-azaadeninyl, 7-deazaguaninyl, 7-deazaadeninyl, 3-deazaguaninyl and 3-deazaadeninyl.

38. The oligomeric compound of claim 23 wherein nn is from about 8 to about 30.

39. The oligomeric compound of claim 23 wherein nn is from about 15 to about 25.

40. The oligomeric compound of claim 23 wherein said conjugate group is a contrast reagent, a cleaving agent, a cell targeting agent, polyethylene glycol, cholesterol, phospholipid, biotin, phenanthroline, phenazine, phenanthridine, anthraquinone, acridine, fluorescein, rhodamine, coumarin, pyrene, retinal or a cyanine dye.

41. An oligomeric compound of formula I wherein:
T₁ is hydrogen, an amino protecting group, —C(O)R₅, substituted or unsubstituted C₁-C₁₀ alkyl, substituted or unsubstituted C₂-C₁₀ alkenyl, substituted or unsubstituted C₂-C₁₀ alkynyl, alkylsulfonyl, arylsulfonyl, a chemical functional group, a reporter group, a conjugate group, a D or L α-amino acid linked via the α-carboxyl group or optionally through the ω-carboxyl group when the amino acid is aspartic acid or glutamic acid or a peptide derived from D, L or mixed D and L amino acids linked through a carboxyl group, wherein the substituent groups are selected from hydroxyl, amino, alkoxy, carboxy, benzyl, phenyl, nitro, thiol, thioalkoxy, halogen, alkyl, aryl, alkenyl and alkynyl;
T₂ is —OH, —N(Z₁)Z₂, R₅, D or L α-amino acid linked via the α-amino group or optionally through the c-amino group when the amino acid is lysine or ornithine or a peptide derived from D, L or mixed D and L amino acids linked through an amino group, a chemical functional group, a reporter group or a conjugate group;
nn is from 2 to about 50;
each Bx is, independently, an optionally protected heterocyclic base moiety wherein at least one of said heterocyclic base moieties has formula VIII: 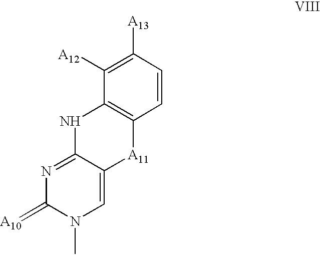
wherein
A₁₀ is S; and A₁₁ is CH₂, O or S; or
A₁₀ is O and A₁₁ is CH₂;
one of A₁₂ and A₁₃ is hydrogen and the other of A₁₂ and A₁₃ is a group of formula: 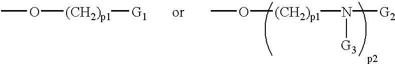
wherein:
G₁ is —CN, —OA₂₀, —SA₂₀, —N(H)A₂₀, —ON(H)A₂₀ or —C(═NH)N(H)A₂₀;
G₂ is H, —NHA₂₀, —C(═O)N(H)A₂₀, —C(═S)N(H)A₂₀ or —C(═NH)N(H)A₂₀,
each G₃ is, independently, H or an amino protecting group;
A₂₀ is H, a protecting group, substituted or unsubstituted C₁-C₁₀ alkyl, acetyl, benzyl, —(CH₂)ₚ₃NH₂, —(CH₂)ₚ₃N(H)G₃, a D or L α-amino acid, or a peptide derived from D, L or racemic ac-amino acids;
each R₅ is a carbonyl protecting group;
each p1 is, independently, from 2 to about 6;
p2 is from 1 to about 3; and
p3 is from 1 to about 4.

42. The oligomeric compound of claim 41 wherein:
A₁₃ is H;
A₁₂ is —O—(CH₂)₂—N(H)G₄, —O—(CH₂)₂—ON(H)G₄ or —O—(CH₂)₂—C(═NH)N(H)G₄, —O—(CH₂)₃—C(═NH)N(H)G₄, —O—(CH₂)₂—C(═O)N(H)G₄, —O—(CH₂)₂—C(═S)N(H)G₄ or —O—(CH₂)₂—N(H)C(═NH)N(H)G₄; and
G₄ is hydrogen, an amino protecting group or C₁-C₁₀ alkyl.

43. The oligomeric compound of claim 42 wherein A₁₀ is S.

44. The oligomeric compound of claim 43 wherein A₁₁ is O.

45. The oligomeric compound of claim 41 wherein T₁ is hydrogen, an amino protecting group, a reporter group, a D or L amino acid or a peptide.

46. The oligomeric compound of claim 45 wherein said D or L amino acid is lysine or glutamic acid.

47. The oligomeric compound of claim 41 wherein T₂ is —OH, —(Z₁)Z₂, R₅, a D or L amino acid or a peptide.

48. The oligomeric compound of claim 41 wherein each carbonyl protecting group is, independently, substituted or unsubstituted C₁-C₁₀ alkyl, trifluoromethyl, cyanoethyloxy, methoxy, ethoxy, t-butoxy, allyloxy, 9-fluorenylmethoxy, 2-(trimethylsilyl)-ethoxy, 2,2,2-trichloroethoxy, benzyloxy, butyryl, iso-butyryl, phenyl or aryl.

49. The oligomeric compound of claim 41 wherein each Bx is independently selected from the group consisting of a radical of formula VIII, adeninyl, guaninyl, thyminyl, cytosinyl, uracilyl, 5-methylcytosinyl (5-me-C), 5-hydroxymethyl cytosinyl, xanthinyl, hypoxanthinyl, 2-aminoadeninyl, alkyl derivatives of adeninyl and guaninyl, 2-thiouracilyl, 2-thiothyminyl, 2-thiocytosinyl, 5-halouracilyl, 5-halocytosinyl, 5-propynyl uracilyl, 5-propynyl cytosinyl, 6-azo uracilyl, 6-azo cytosinyl, 6-azo thyminyl, 5-uracilyl (pseudouracil), 4-thiouracilyl, 8-substituted adeninyls and guaninyls, 5-substituted uracilyls and cytosinyls, 7-methylguaninyl, 7-methyladeninyl, 8-azaguaninyl, 8-azaadeninyl, 7-deazaguaninyl, 7-deazaadeninyl, 3-deazaguaninyl and 3-deazaadeninyl.

50. The oligomeric compound of claim 41 wherein nn is from about 8 to about 30.

51. The oligomeric compound of claim 41 wherein nn is from about 15 to about 25.

52. The oligomeric compound of claim 41 wherein said conjugate group is a contrast reagent, a cleaving agent, a cell targeting agent, polyethylene glycol, cholesterol, phospholipid, biotin, phenanthroline, phenazine, phenanthridine, anthraquinone, acridine, fluorescein, rhodamine, coumarin, pyrene, retinal or a cyanine dye.

53. An oligomeric compound of formula I wherein:
T₁ is hydrogen, an amino protecting group, —C(O)R₅, substituted or unsubstituted C₁-C₁₀ alkyl, substituted or unsubstituted C₂-C₁₀ alkenyl, substituted or unsubstituted C₂-C₁₀ alkynyl, alkylsulfonyl, arylsulfonyl, a chemical functional group, a reporter group, a conjugate group, a D or L α-amino acid linked via the α-carboxyl group or optionally through the ()—carboxyl group when the amino acid is aspartic acid or glutamic acid or a peptide derived from D, L or mixed D and L amino acids linked through a carboxyl group, wherein the substituent groups are selected from hydroxyl, amino, alkoxy, carboxy, benzyl, phenyl, nitro, thiol, thioalkoxy, halogen, alkyl, aryl, alkenyl and alkynyl;
T₂ is —OH, —N(Z₁)Z₂, R₅, D or L α-amino acid linked via the α-amino group or optionally through the ω-amino group when the amino acid is lysine or ornithine or a peptide derived from D, L or mixed D and L amino acids linked through an amino group, a chemical functional group, a reporter group or a conjugate group;
nn is from 2 to about 50;
each Bx is, independently, an optionally protected heterocyclic base moiety wherein at least one of said heterocyclic base moieties has formula XVI: 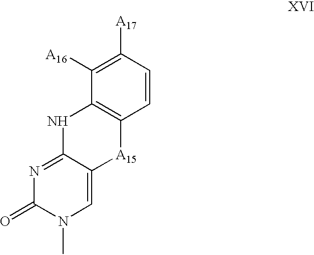
wherein
A₁₅ is O or S; and
A₁₆ is selected from the group consisting of —O—(CH₂)ₚ₁C(═NH)N(H)A₂₀, —O—(CH₂)ₚ₁N(H)—C(═O)N(H)A₂₀ or —O—(CH₂)ₚ₁N(H)—C(═S)N(H)A₂₀ and A₁₇ is H;
or A₁₆ is H and A₁₇ is a group of formula: 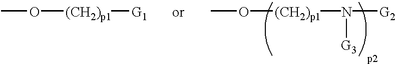
wherein:
G₁ is —CN, —OA₂₀, —SA₂₀, —N(H)A₂₀, —ON(H)A₂₀ or —C(═NH)N(H)A₂₀;
G₂ is H, —NHA₂₀, —C(═O)N(H)A₂₀, —C(═S)N(H)A₂₀ or —C(═NH)N(H)A₂₀,
each G₃ is, independently, H or an amino protecting group;
A₂₀ is H, a protecting group, substituted or unsubstituted C₁-C₁₀ alkyl, acetyl, benzyl, —(CH₂)ₚ₃N(H)G₃, a D or L α-amino acid, or a peptide derived from D, L or racemic α-amino acids;
each R₅ is carbonyl protecting group;
each p1 is from 2 to about 6;
p2 is from 1 to about 3; and
p3 is from 1 to about 4.

54. The oligomeric compound of claim 53 wherein:
A₁₆ is H;
A₁₇ is —O—(CH₂)₂—N(H)G₄, —O—(CH₂)₂—ON(H)G₄ or —O—(CH₂)₂—C(═NH)N(H)G₄, —O—(CH₂)₃—C(═NH)N(H)G₄, —O—(CH₂)₂—C(═O)N(H)G₄, —O—(CH₂)₂—C(═S)N(H)G₄ or —O—(CH₂)₂—N(H)C(═NH)N(H)G₄; and
G₄ is hydrogen, an amino protecting group or C₁-C₁₀ alkyl.

55. The oligomeric compound of claim 53 wherein A₁₅ is S.

56. The oligomeric compound of claim 53 wherein A₁₅ is O.

57. The oligomeric compound of claim 53 wherein n is from about 8 to about 30.

58. The oligomeric compound of claim 53 wherein n is from about 15 to about 25.

59. The oligomeric compound of claim 53 wherein T₁ is hydrogen, an amino protecting group, a reporter group, a D or L amino acid or a peptide.

60. The oligomeric compound of claim 59 wherein said D or L amino acid is lysine or glutamic acid.

61. The oligomeric compound of claim 53 wherein T₂ is —OH, —(Z₁)Z₂, R₅, a D or L amino acid or a peptide.

62. The oligomeric compound of claim 53 wherein each carboxylic protecting group is, independently, substituted or unsubstituted C₁-C₁₀ alkyl, trifluoromethyl, cyanoethyloxy, methoxy, ethoxy, t-butoxy, allyloxy, 9-fluorenylmethoxy, 2-(trimethylsilyl)-ethoxy, 2,2,2-trichloroethoxy, benzyloxy, butyryl, iso-butyryl, phenyl or aryl.

63. The oligomeric compound of claim 53 wherein each Bx is independently selected from the group consisting of a radical of formula XVI, adeninyl, guaninyl, thyminyl, cytosinyl, uracilyl, 5-methylcytosinyl (5-me-C), 5-hydroxymethyl cytosinyl, xanthinyl, hypoxanthinyl, 2-aminoadeninyl, alkyl derivatives of adeninyl and guaninyl, 2-thiouracilyl, 2-thiothyminyl, 2-thiocytosinyl, 5-halouracilyl, 5-halocytosinyl, 5-propynyl uracilyl, 5-propynyl cytosinyl, 6-azo uracilyl, 6-azo cytosinyl, 6-azo thyminyl, 5-uracilyl (pseudouracil), 4-thiouracilyl, 8-substituted adeninyls and guaninyls, 5-substituted uracilyls and cytosinyls, 7-methylguaninyl, 7-methyladeninyl, 8-azaguaninyl, 8-azaadeninyl, 7-deazaguaninyl, 7-deazaadeninyl, 3-deazaguaninyl and 3-deazaadeninyl.

64. The oligomeric compound of claim 53 wherein nn is from about 8 to about 30.

65. The oligomeric compound of claim 53 wherein nn is from about 15 to about 25.

66. The oligomeric compound of claim 53 wherein said conjugate group is a contrast reagent, a cleaving agent, a cell targeting agent, polyethylene glycol, cholesterol, phos- pholipid, biotin, phenanthroline, phenazine, phenanthridine, anthraquinone, acridine, fluorescein, rhodamine, coumarin, pyrene, retinal or a cyanine dye.

67. An oligomeric compound having one of formulas X, XI, XII, XIII, XIV or XV: 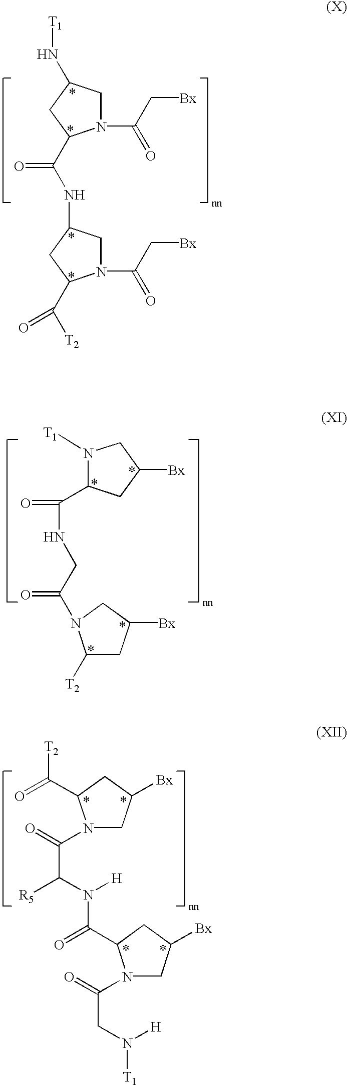 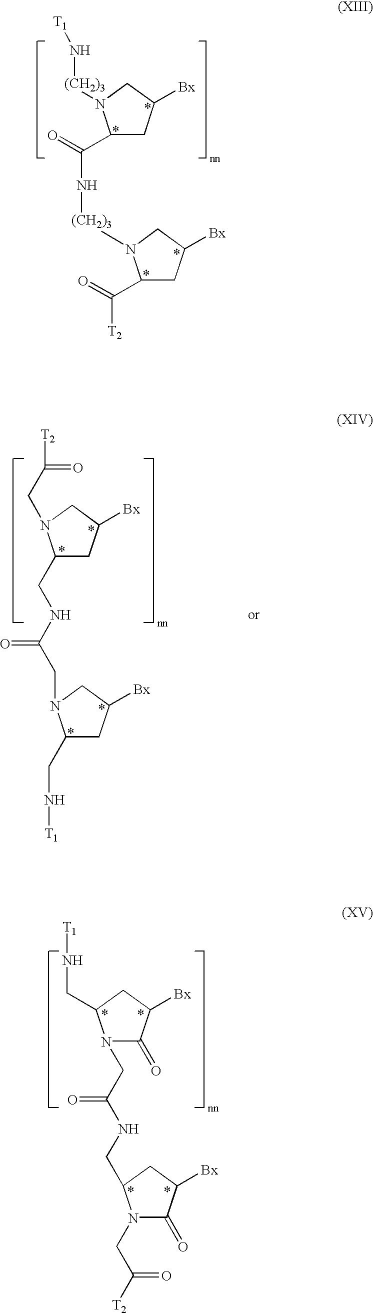
wherein:
T₁ is hydrogen, an amino protecting group, —C(O)R₅, substituted or unsubstituted C₁-C₁₀ alkyl, substituted or unsubstituted C₂-C₁₀ alkenyl, substituted or unsubstituted C₂-C₁₀ alkynyl, alkylsulfonyl, arylsulfonyl, a chemical functional group, a reporter group, a conjugate group, a D or L α-amino acid linked via the α-carboxyl group or optionally through the ω-carboxyl group when the amino acid is aspartic acid or glutamic acid or a peptide derived from D, L or mixed D and L amino acids linked through a carboxyl group, wherein the substituent groups are selected from hydroxyl, amino, alkoxy, carboxy, benzyl, phenyl, nitro, thiol, thioalkoxy, halogen, alkyl, aryl, alkenyl and alkynyl;
T₂ is —OH, —N(Z₁)Z₂, R₅, D or L α-amino acid linked via the α-amino group or optionally through the ω-amino group when the amino acid is lysine or ornithine or a peptide derived from D, L or mixed D and L amino acids linked through an amino group, a chemical functional group, a reporter group or a conjugate group;
nn is from 2 to about 50;
each chiral ring carbon having an asterick (*) is prepared having R, S or mixed R and S configuration;
each Bx is, independently, an optionally protected heterocyclic base moiety wherein at least one of said heterocyclic base moieties has one of formulas II or III: 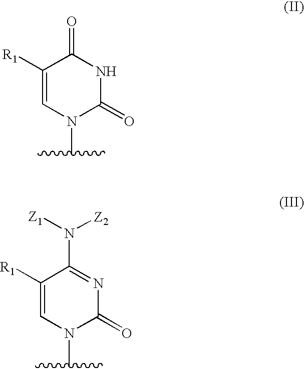
wherein:
R₁ is —CH₂—Q₂, —C≡C—Q₂, —CH₂(CH₂)ₙ—Q₃, or —CH═CH—C(═O)—Q₄;
Q₁ is —N₃, —CN, —N(Z₁)₂, —N(Z₁)—(CH₂)ₙ—C(═NH₂)—N(H)—Z₃, —N(Z₁)—C(═J)—N(H)—Z₅, —L—(CH₂)ₙ—C(═O)Z₃, —L—(CH₂)ₙ—L—Z₃, —L—(CH₂)ₙ—N(H)Z₁, —L—(CH₂)ₙ—N(Z₁)—(CH₂)ₙ—N(H)Z₁, —L—(CH₂)ₙ—C(═NH)N(Z₁)Z₃ or —L—(CH₂)ₙ—N(Z₁)—C(═J)—N(H)Z₃;
Q₂ is H, C₁-C₆ alkyl, —C(═O)—N(H)Z₁, —C(═O)—O—CH₂—CH₃, C(═O)—O—benzyl, —C(═O)—Z₄, —CH₂—O—Q₆, —CH₂—C(═NH)—N(H)—Z₃, —CH₂—N(H)—Z₂,—CH₂—N(H)—C(═O)—CF₃, —CH₂—N(H)Z₁, —CH₂—N(H)—C(═NH)—N(H)—Z₃, —CH₂—O—N(H)—C(═O)—(CH₂)ₙ—Q₅, —C(═O)—N(H)—(CH₂)ₙ—Q₅ or —CH₂—N(H)—C(═O)—(CH₂)ₙ—Q₅;
Q₃ is hydrogen, —O—C₁-C₆ alkyl, —N(H)—Z₁, —N(H)—Z₂, —N(H)—C(═O)—CF₃, —N(H)—C(═NH)—N(H)Z₁, —O—Q₆, —N(H)—C(═O)—(CH₂)ₙ—Q₅, —O—N(H)—C(═O)—(CH₂)ₙ—Q₅, —C(═O)—N(H)—(CH₂)ₙ—Q₅ or —C(═O)—Q₇;
Q₄ is is Z₄, —L—Z₃, —N(H)Z₃, —C(═NH)—N(H)Z₃, —N(H)—C(═J) N(H)Z₃, N(Z₁)—(CH₂)ₙ—N(H)Z₃, —C(═O)—N(H)—(CH₂)ₙ—C(═NH)—N(H)Z₃, —C(═O)—N(H)—(CH₂)ₙ—N(H)—C(═J)—N(H)Z₃ or —C(═O)—N(H)—(CH₂)ₙ—N(Z₁)—(CH₂)ₙ—N(H)Z₁;
Q₅ is —L—Z₃, —N(H)Z₃, —C(═NH)—N(H)Z₃, —N(H)—C(═J) N(H)Z₃ or N(Z₁)—(CH₂)ₙ—N(H)Z₃;
Q₆ is hydrogen, —N(H)Z₁, —N(H)Z₂, benzyl, benzoyl, —C(═O)—(CH₂)ₙ—H or phthalimido;
Q₇ is —OH, —O—C₁-C₆ alkyl, —O-benzyl, —Z₄, —N(H)Z₁,
each L is O or S;
each J is O, S or NH;
each n is from 1 to 6;
Z₁ is hydrogen, C₁-C₆ alkyl, or an amino protecting group;
Z₂ is hydrogen, C₁-C₆ alkyl, an amino protecting group, —C(═O)—(CH₂)ₙ—J—Z₃, a D or L α-amino acid linked via the α-carboxyl group or optionally through the ω-carboxyl group when the amino acid is aspartic acid or glutamic acid or a peptide derived from D, L or mixed D and L amino acids linked through a carboxyl group;
Z₃ is hydrogen, an amino protecting group, —C₁-C₆ alkyl, —C(═O)—CH₃, benzyl, benzoyl, or —(CH₂)ₙ—N(H)Z₁,;
Z₄ is a D or L α-amino acid linked via the α-amino group or optionally through the ω-amino group when the amino acid is lysine or ornithine or a peptide derived from D, L or mixed D and L amino acids linked through an amino group;
Z₅ is hydrogen, an amino protecting group or —C(═O)—(CH₂)ₙ—J—Z₃; and
each R₅ is a carbonyl protecting group.

68. The oligomeric compound of claim 67 wherein R₁ is —CH₂—Q₁.

69. The oligomeric compound of claim 68 wherein Q₁ is —N₃, —CN, —N(Z₁)Z₂, —N(Z₁)—(CH₂)ₙ—C(═NH)—N(H)—Z₃, —N(Z₁)—C(═J)—N(H)—Z₅, —L—(CH₂)ₙ—C(═O)Z₃, —L—(CH₂)ₙ—L—Z₃, —L—(CH₂)ₙ—N(H)Z₁, —L—(CH₂)ₙ—N(Z₁)—(CH₂)ₙ—N(H)Z₁, —L—(CH₂)ₙ—C(═NH)N(Z₁)Z₃ or —L—(CH₂)ₙ—N(Z₁)—C(═J)—N(H)Z₃.

70. The oligomeric compound of claim 67 wherein Z₁, Z₂, Z₃, Z₄ and Z₅ are each independently hydrogen, methyl or an amino protecting group,

71. The oligomeric compound of claim 67 wherein each n is independently from 1 to about 3.

72. The oligomeric compound of claim 67 wherein R₁ is —C≡C—Q₂.

73. The oligomeric compound of claim 72 wherein Q₂ is H, methyl, ethyl, —C(═O)—N(H)Z₁, —CH₂—N(H)—Z₂ or —CH₂—N(H)—C(═NH)—N(H)—Z₅.

74. The oligomeric compound of claim 67 wherein R₁ is —CH₂—(CH₂)ₙ—Q₃.

75. The oligomeric compound of claim 74 wherein each Q₃ is hydrogen, —O—CH₃, —O—CH₂CH₃, —N(H)—Z₁, —N(H)—Z₂, —N(H)—C(═O)—CF₃ or —N(H)—C(═NH)—N(H)Z₁.

76. The oligomeric compound of claim 74 wherein Q₃ is —N(H)—C(═O)—(CH₂)ₙ—Q₅, —O—N(H)—C(═O)—(CH₂)ₙ—Q₅ or —C(═O)—N(H)—(CH₂)ₙ—Q₅ and Q₅ is —N(H)Z₃, —C(═NH)—N(H)Z₃ or —N(H)—C(═J) N(H)Z₃.

77. The oligomeric compound of claim 74 wherein Q₃ is —O—Q₆ and Q₆ is hydrogen, —N(H)Z₁ or —N(H)Z₂.

78. The oligomeric compound of claim 67 wherein each R₁ is —CH═CH—C(═O)—Q₄.

79. The oligomeric compound of claim 78 wherein Q₄ is —OH, —N(H)Z₃, —C₁-C₆ alkyl, —O—C₁-C₆ alkyl, —O-benzyl or —N(H)—(CH₂)ₙ—Q₅.

80. The oligomeric compound of claim 79 wherein Q₄ is —N(H)Z₃ and Z₃ is Hydrogen or C₁-C₆ alkyl.

81. The oligomeric compound of claim 67 wherein each carbonyl protecting group is, independently, substituted or unsubstituted C₁-C₁₀ alkyl, trifluoromethyl, cyanoethyloxy, methoxy, ethoxy, t-butoxy, allyloxy, 9-fluorenylmethoxy, 2—(trimethylsilyl)-ethoxy, 2,2,2-trichloroethoxy, benzyloxy, butyryl, iso-butyryl, phenyl or aryl.

82. The oligomeric compound of claim 67 wherein T₁ is hydrogen, an amino protecting group, a reporter group or a D or L amino acid or a peptide.

83. The oligomeric compound of claim 82 wherein said D or L amino acid is lysine or glutamic acid.

84. The oligomeric compound of claim 67 wherein T₂ is —OH, —N(Z₁)Z₂, R₅ or a D or L amino acid or a peptide.

85. The oligomeric compound of claim 67 wherein said conjugate group is a contrast reagent, a cleaving agent, a cell targeting agent, polyethylene glycol, cholesterol, phospholipid, biotin, phenanthroline, phenazine, phenanthridine, anthraquinone, acridine, fluorescein, rhodamine, coumarin, pyrene, retinal or a cyanine dye.

86. The oligomeric compound of claim 67 wherein each Bx is independently selected from the group consisting of a radical of formula II, formula III, adeninyl, guaninyl, thyminyl, cytosinyl, uracilyl, 5-methylcytosinyl (5-me-C), 5-hydroxymethyl cytosinyl, xanthinyl, hypoxanthinyl, 2-aminoadeninyl, alkyl derivatives of adeninyl and guaninyl, 2-thiouracilyl, 2-thiothyminyl, 2-thiocytosinyl, 5-halouracilyl, 5-halocytosinyl, 5-propynyl uracilyl, 5-propynyl cytosinyl, 6-azo uracilyl, 6-azo cytosinyl, 6-azo thyminyl, 5-uracilyl (pseudouracil), 4-thiouracilyl, 8-substituted adeninyls and guaninyls, 5-substituted uracilyls and cytosinyls, 7-methylguaninyl, 7-methyladeninyl, 8-azaguaninyl, 8-azaadeninyl, 7-deazaguaninyl, 7-deazaadeninyl, 3-deazaguaninyl and 3-deazaadeninyl.

87. The oligomeric compound of claim 67 wherein nn is from about 8 to about 30.

88. The oligomeric compound of claim 67 wherein nn is from about 15 to about 25.

89. The oligomeric compound of claim 67 prepared having substantially pure R or S configuration at each of said chiral ring carbons.

90. The oligomeric compound of claim 67 prepared having essentially equal amounts of R and S configuration at each of said chiral ring carbons.

91. An oligomeric compound having one of formulas X, XI, XII, XIII, XIV or XV: 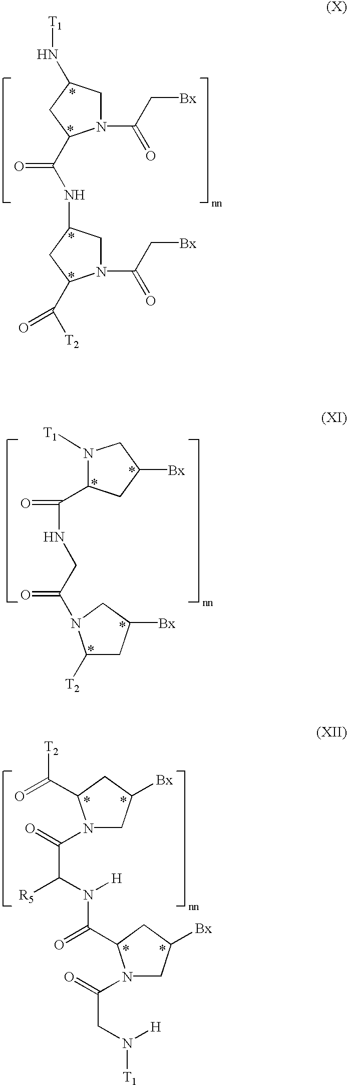 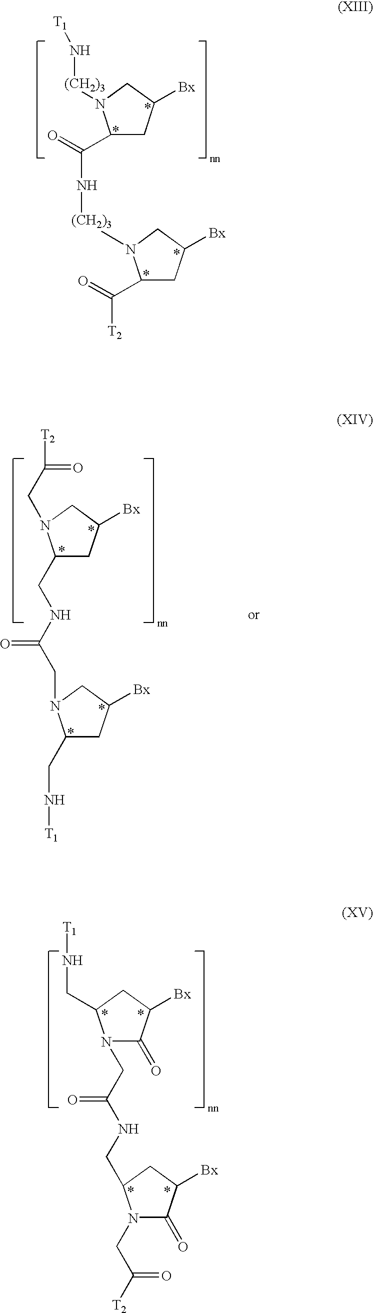
wherein:
T₁ is hydrogen, an amino protecting group, —C(O)R₅, substituted or unsubstituted C₁-C₁₀ alkyl, substituted or unsubstituted C₂-C₁₀ alkenyl, substituted or unsubstituted C₂-C₁₀ alkynyl, alkylsulfonyl, arylsulfonyl, a chemical functional group, a reporter group, a conjugate group, a D or L α-amino acid linked via the α-carboxyl group or optionally through the ω-carboxyl group when the amino acid is aspartic acid or glutamic acid or a peptide derived from D, L or mixed D and L amino acids linked through a carboxyl group, wherein the substituent groups are selected from hydroxyl, amino, alkoxy, carboxy, benzyl, phenyl, nitro, thiol, thioalkoxy, halogen, alkyl, aryl, alkenyl and alkynyl;
T₂ is —OH, —N(Z₁)Z₂, R₅, D or L α-amino acid linked via the α-amino group or optionally through the ω-amino group when the amino acid is lysine or ornithine or a peptide derived from D, L or mixed D and L amino acids linked through an amino group, a chemical functional group, a reporter group or a conjugate group;
nn is from 2 to about 50;
each chiral ring carbon having an asterick (*) is prepared having R, S or mixed R and S configuration;
each Bx is, independently, an optionally protected heterocyclic base moiety wherein at least one of said heterocyclic base moieties has one of formulas V or VI: 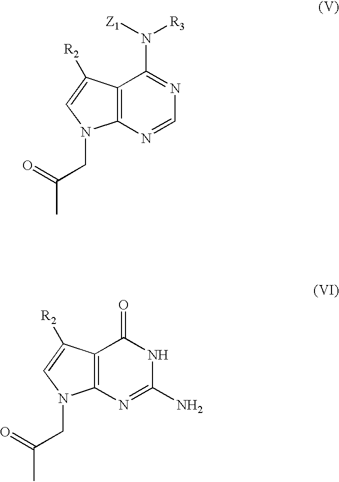
wherein:
R₂ is hydrogen and R₃ is Z₁, —C(═J)—N(H)Z₁, —C(═O)—(CH₂)ₙ—N(H)Z₁, —C(═O)—(CH₂)ₙ—L—Z₉, —(CH₂)ₙ—N(H)Z₁, —(CH₂)ₙ—N(Z₁)—(CH₂)ₙ—N(H)Z₁, —(CH₂)ₙ—N(H)—C(═J)—N(H)Z₃ or —(CH₂)ₙ—C(═NH)—N(H)Z₃;
or R₃ is hydrogen and R₂ is —C≡C—R₄ or —(CH₂)ₘ—R₄;
L is O or S;
J is O, S or NH;
m is from 2 to 6;
each n is from 1 to 6;
R₄ is H, C₁-C₆ alkyl, —CH₂OH, —CH₂—O—Q₆, —CH₂—N(H)—C(═O)—CF₃, —CH₂—N(H)Z₁, —CH₂—N(H)Z₂, —C(═O)—Z₄, —C(═O)—N(H)—(CH₂)ₙ—Q₅, —CH₂—N(H)—C(═O)—(CH₂)ₙQ₅ or —CH₂—O—N(H)—C(═O)—(CH₂)ₙ—Q₅;
Q₅ is —L—Z₃, —N(H)Z₁, —C(═NH)—N(H)Z₃, —N(H)—C(═J) N(H)Z₃ or —N(Z₁)—(CH₂)ₙ—N(H)Z₁;
Q₆ is —N(H)Z₁, —N(H)Z₂, benzyl, benzoyl, —C(═O)—(CH₂)ₙ—CH₃ or phthalimido;
Z₁ is hydrogen, C₁-C₆ alkyl, or an amino protecting group;
Z₂ is hydrogen, C₁-C₆ alkyl, an amino protecting group, —C(═O)—(CH₂)ₙ—J—Z₃, a D or L α-amino acid linked via the α-carboxyl group or optionally through the ω-carboxyl group when the amino acid is aspartic acid or glutamic acid or a peptide derived from D, L or mixed D and L amino acids linked through a carboxyl group;
Z₃ is hydrogen, an amino protecting group, —C₁-C₆ alkyl, —C(═O)—CH₃, benzyl, benzoyl, or —(CH₂)ₙ—N(H)Z₁;
Z₄ is —OH, C₁-C₆ alkyl, benzyl, —N(H)Z₁, a D or L α-amino acid linked via the α-amino group or optionally through the ω-amino group when the amino acid is lysine or ornithing or a peptide derived from D, L or mixed D and L amino acids linked through an amino group;
Z₉ is hydrogen, —C₁-C₆ alkyl, —C(═O)—CH₃, benzyl or a D or L α-amino acid linked via the α-carboxyl group or optionally through the ω-carboxyl group when the amino acid is aspartic acid or glutamic acid or a peptide derived from D, L or mixed D and L amino acids linked through a carboxyl group; and
each R₅ is a carbonyl protecting group.

92. The oligomeric compound of claim 91 wherein R₂ is hydrogen and R₃ is Z₁, —C(═J)—N(H)Z₁ or —(CH₂)ₙ—C(═NH)—N(H)Z₃.

93. The oligomeric compound of claim 91 wherein R₃ is hydrogen and R₂ is —C≡C—R₄ or —(CH₂)ₘ—R₄.

94. The oligomeric compound of claim 93 wherein R₄ is H, C₁-C₃ alkyl, —CH₂OH, —CH₂—O—Q₆, —CH₂—N(H)Z₂ or —C(═O)—Z₄.

95. The oligomeric compound of claim 93 wherein R₄ is —C(═O)—Z₄, —C(═O)—N(H)—(CH₂)ₙ—Q₅, —CH₂—N(H)—C(═O)—(CH₂)ₙQ₅ or —CH₂—O—N(H)—C(═O)—(CH₂)ₙ—Q₅ and Q₅ is —N(H)Z₁ or —C(═NH)—N(H)Z₃.

96. The oligomeric compound of claim 93 wherein R₄ is —CH₂—O—Q₆ and Q₆ is —N(H)Z₂, —C(═O)—(CH₂)ₙ—CH₃ or phthalimido.

97. The oligomeric compound of claim 91 wherein T₂ is —N(Z₁)Z₂ and Z₂ is hydrogen, C₁-C₃ alkyl, an amino protecting group.

98. The oligomeric compound of claim 91 wherein R₃ is —(CH₂)ₙ—N(H)—C(═J)—N(H)Z₃ or —(CH₂)ₙ—C(═NH)—N(H)Z₃ and Z₃ is hydrogen, an amino protecting group, —C₁-C₃ alkyl or —C(═O)—CH₃.

99. The oligomeric compound of claim 93 wherein R₄ is —C(═O)—Z₄ and Z₄ is —OH, C₁-C₃ alkyl, benzyl or —N(H)Z₁.

100. The oligomeric compound of claim 91 wherein R₂ is —C(═O)—(CH₂)ₙ—L—Z₉ and Z₉ is hydrogen, —C₁-C₃ alkyl or —C(═O)—CH₃.

101. The oligomeric compound of claim 91 wherein each carbonyl protecting group is, independently, substituted or unsubstituted C₁-C₁₀ alkyl, trifluoromethyl, cyanoethyloxy, methoxy, ethoxy, t-butoxy, allyloxy, 9-fluorenylmethoxy, 2—(trimethylsilyl)-ethoxy, 2,2,2-trichloroethoxy, benzyloxy, butyryl, iso-butyryl, phenyl or aryl.

102. The oligomeric compound of claim 91 wherein T₁ is hydrogen, an amino protecting group, a reporter group, a D or L amino acid or a peptide.

103. The oligomeric compound of claim 102 wherein said D or L amino acid is lysine or glutamic acid.

104. The oligomeric compound of claim 91 wherein T₂ is —OH, —(Z₁)Z₂, R₅, a D or L amino acid or a peptide.

105. The oligomeric compound of claim 91 wherein each Bx is independently selected from the group consisting of a radical of formula V, formula VI, adeninyl, guaninyl, thyminyl, cytosinyl, uracilyl, 5-methylcytosinyl (5-me-C), 5-hydroxymethyl cytosinyl, xanthinyl, hypoxanthinyl, 2-aminoadeninyl, alkyl derivatives of adeninyl and guaninyl, 2-thiouracilyl, 2-thiothyminyl, 2-thiocytosinyl, 5-halouracilyl, 5-halocytosinyl, 5-propynyl uracilyl, 5-propynyl cytosinyl, 6-azo uracilyl, 6-azo cytosinyl, 6-azo thyminyl, 5-uracilyl (pseudouracil), 4-thiouracilyl, 8-substituted adeninyls and guaninyls, 5-substituted uracilyls and cytosinyls, 7-methylguaninyl, 7-methyladeninyl, 8-azaguaninyl, 8-azaadeninyl, 7-deazaguaninyl, 7-deazaadeninyl, 3-deazaguaninyl and 3-deazaadeninyl.

106. The oligomeric compound of claim 91 wherein un is from about 8 to about 30.

107. The oligomeric compound of claim 91 wherein nn is from about 15 to about 25.

108. The oligomeric compound of claim 91 wherein said conjugate group is a contrast reagent, a cleaving agent, a cell targeting agent, polyethylene glycol, cholesterol, phospholipid, biotin, phenanthroline, phenazine, phenanthridine, anthraquinone, acridine, fluorescein, rhodamine, coumarin, pyrene, retinal or a cyanine dye.

109. The oligomeric compound of claim 91 prepared having substantially pure R or S configuration at each of said chiral ring carbons.

110. The oligomeric compound of claim 91 prepared having essentially equal amounts of R and S configuration at each of said chiral ring carbons.

111. An oligomeric compound having one of formulas X, XI, XII, XIII, XIV or XV: 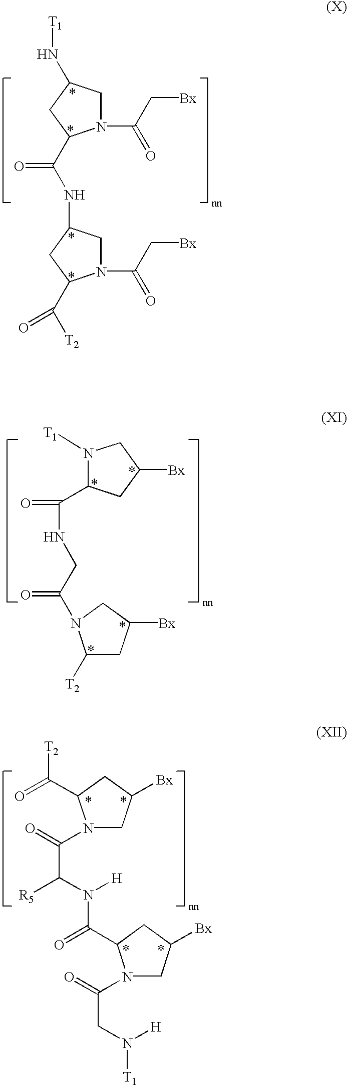 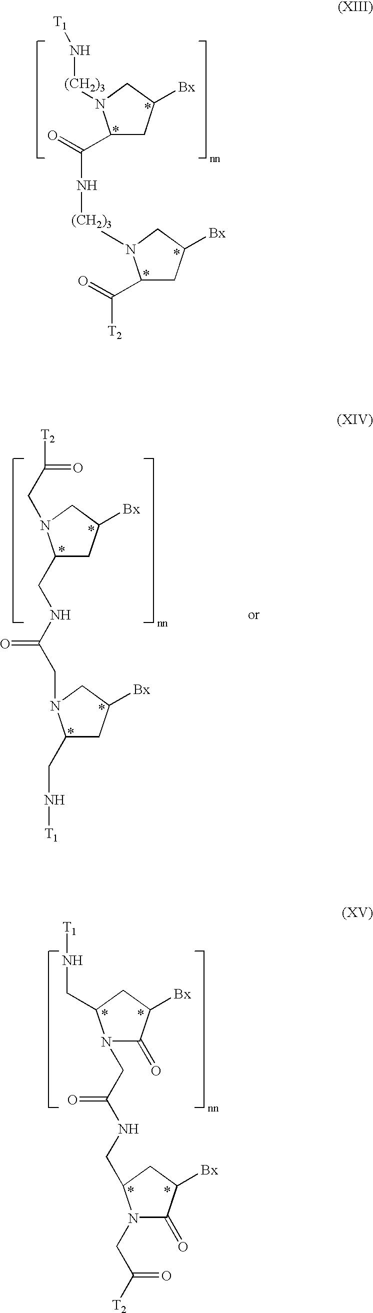
wherein:
T₁ is hydrogen, an amino protecting group, —C(O)R₅, substituted or unsubstituted C₁-C₁₀ alkyl, substituted or unsubstituted C₂-C₁₀ alkenyl, substituted or unsubstituted C₂-C₁₀ alkynyl, alkylsulfonyl, arylsulfonyl, a chemical functional group, a reporter group, a conjugate group, a D or L α-amino acid linked via the α-carboxyl group or optionally through the ω-carboxyl group when the amino acid is aspartic acid or glutamic acid or a peptide derived from D, L or mixed D and L amino acids linked through a carboxyl group, wherein the substituent groups are selected from hydroxyl, amino, alkoxy, carboxy, benzyl, phenyl, nitro, thiol, thioalkoxy, halogen, alkyl, aryl, alkenyl and alkynyl;
T₂ is —OH, —N(Z₁)Z₂, R₅, D or L α-amino acid linked via the α-amino group or optionally through the ω-amino group when the amino acid is lysine or ornithine or a peptide derived from D, L or mixed D and L amino acids linked through an amino group, a chemical functional group, a reporter group or a conjugate group;
nn is from 2 to about 50;
each chiral ring carbon having an asterick (*) is prepared having R, S or mixed R and S configuration;
each Bx is, independently, an optionally protected heterocyclic base moiety wherein at least one of said heterocyclic base moieties has formula VIII: 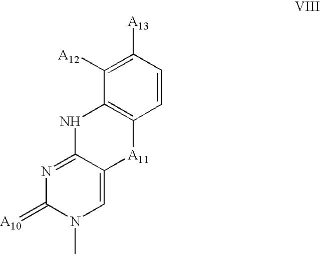
wherein
A₁₀ is O or S;
A₁₁ is CH₂, N—CH₃, O or S;
each A₁₂ and A₁₃ is hydrogen or one of A₁₂ and A₁₃ is hydrogen and the other of A₁₂ and A₁₃ is a group of formula: 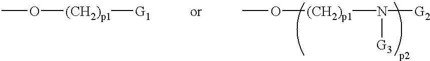
wherein:
G₁ is —CN, —OA₂₀, —SA₂₀, —N(H)A₂₀, —ON(H)A₂₀ or —C(═NH)N(H)A₂₀;
G₂ is H, —NHA₂₀, —C(═O)N(H)A₂₀, —C(═S)N(H)A₂₀ or —C(═NH)N(H)A₂₀, each G₃ is, independently, H or an amino protecting group;
A₂₀ is H, a protecting group, substituted or unsubstituted C₁-C₁₀ alkyl, acetyl, benzyl, —(CH₂)ₚ₃NH₂, —(CH₂)ₚ₃N(H)G₃, a D or L α-amino acid, or a peptide derived from D, L or racemic α-amino acids;
each R₅ is a carbonyl protecting group;
each p1 is, independently, from 2 to about 6;
p2 is from 1 to about 3; and
p3 is from 1 to about 4.

112. The oligomeric compound of claim 111 wherein:
A₁₃ is H;
A₁₂ is —O—(CH₂)₂—N(H)G₄, —O—(CH₂)₂—ON(H)G₄ or —O—(CH₂)₂—C(═NH)N(H)G₄, —O—(CH₂)₃—C(═NH)N(H)G₄, —O—(CH₂)₂—C(═O)N(H)G₄, —O—(CH₂)₂—C(═S)N(H)G₄ or —O—(CH₂)₂—N(H)C(═NH)N(H)G₄; and
G₄ is hydrogen, an amino protecting group or C₁-C₁₀ alkyl.

113. The oligomeric compound of claim 112 wherein A₁₀ is S.

114. The oligomeric compound of claim 113 wherein A₁₁ is O.

115. The oligomeric compound of claim 111 wherein T₁ is hydrogen, an amino protecting group, a reporter group, a D or L amino acid or a peptide.

116. The oligomeric compound of claim 115 wherein said D or L amino acid is lysine or glutamic acid.

117. The oligomeric compound of claim 111 wherein T₂ is —OH, —(Z₁)Z₂, R₅, a D or L amino acid or a peptide.

118. The oligomeric compound of claim 111 wherein each carbonyl protecting group is, independently, substituted or unsubstituted C₁-C₁₀ alkyl, trifluoromethyl, cyanoethyloxy, methoxy, ethoxy, t-butoxy, allyloxy, 9-fluorenylmethoxy, 2-(trimethylsilyl)-ethoxy, 2,2,2-trichloroethoxy, benzyloxy, butyryl, iso-butyryl, phenyl or aryl.

119. The oligomeric compound of claim 111 wherein each Bx is independently selected from the group consisting of a radical of formula VIII, adeninyl, guaninyl, thyminyl, cytosinyl, uracilyl, 5-methylcytosinyl (5-me-C), 5-hydroxymethyl cytosinyl, xanthinyl, hypoxanthinyl, 2-aminoadeninyl, alkyl derivatives of adeninyl and guaninyl, 2-thiouracilyl, 2-thiothyminyl, 2-thiocytosinyl, 5-halouracilyl, 5-halocytosinyl, 5-propynyl uracilyl, 5-propynyl cytosinyl, 6-azo uracilyl, 6-azo cytosinyl, 6-azo thyminyl, 5-uracilyl (pseudouracil), 4-thiouracilyl, 8-substituted adeninyls and guaninyls, 5-substituted uracilyls and cytosinyls, 7-methylguaninyl, 7-methyladeninyl, 8-azaguaninyl, 8-azaadeninyl, 7-deazaguaninyl, 7-deazaadeninyl, 3-deazaguaninyl and 3-deazaadeninyl.

120. The oligomeric compound of claim 111 wherein nn is from about 8 to about 30.

121. The oligomeric compound of claim 111 wherein nn is from about 15 to about 25.

122. The oligomeric compound of claim 111 wherein said conjugate group is a contrast reagent, a cleaving agent, a cell targeting agent, polyethylene glycol, cholesterol, phospholipid, biotin, phenanthroline, phenazine, phenanthridine, anthraquinone, acridine, fluorescein, rhodamine, coumarin, pyrene, retinal or a cyanine dye.

123. The oligomeric compound of claim 111 prepared having substantially pure R or S configuration at each of said chiral ring carbons.

124. The oligomeric compound of claim 111 prepared having essentially equal amounts of R and S configuration at each of said chiral ring carbons.
